(19) United States
(12) Patent Application Publication
(10) Pub. No.: US 2022/0229044 A1
(43) Pub. Date: Jul. 21, 2022

(54) IN SITU CELL SCREENING METHODS AND SYSTEMS

(71) Applicants: THE BROAD INSTITUTE, INC., Cambridge, MA (US); MASSACHUSETTS INSTITUTE OF TECHNOLOGY, Cambridge, MA (US)

(72) Inventors: David Feldman, Cambridge, MA (US); Paul Blainey, Cambridge, MA (US); Avtar Singh, Cambridge, MA (US); Rebecca Carlson, Cambridge, MA (US)

(21) Appl. No.: 17/055,502

(22) PCT Filed: May 14, 2019
(86) PCT No.: PCT/US2019/032308

§ 371 Date: Nov. 13, 2020

Related U.S. Application Data
(60) Provisional application No. 62/702,209, filed on Jul. 23, 2018.
(60) Provisional application No. 62/671,301, filed on May 14, 2018.

(51) Int. Cl. G01N 33/50; C12N 15/11; C12N 15/86; C12Q 1/6869

(52) U.S. Cl. CPC G01N 33/5026; C12N 15/11; C12N 15/86; C12N 2320/12; C12N 2310/20; C12N 2740/15043; C12Q 1/6869

(57) ABSTRACT

The subject matter disclosed herein is generally directed to methods and systems for screening phenotypes associated with genetic elements and identifying genetic elements at the single-cell level using optical barcodes. A major advantage offered by this approach is the ability to screen for any cellular phenotype that can be identified by high-resolution microscopy—including live-cell phenotypes, protein localization, or highly multiplexed expression profile and mRNA localization in conjunction with a large array of genetic elements applied as a pool in a single test volume.

### CROSS REFERENCE TO RELATED APPLICATIONS

This application is a U.S. national phase entry of International Application No. PCT/US2019/032308 filed May 14, 2019 which claims the benefit of U.S. Provisional Application No. 62/671,301 filed May 14, 2018 and U.S. Provisional Application No. 62/702,209 filed Jul. 23, 2018. The entire contents of the above-identified applications are hereby fully incorporated herein by reference.

### STATEMENT REGARDING FEDERALLY SPONSORED RESEARCH

This invention was made with government support under Grant Nos. HG009280 and HG006193 awarded by the National Institutes of Health. The government has certain rights in the invention.

### REFERENCE TO AN ELECTRONIC SEQUENCE LISTING

The contents of the electronic sequence listing (BROD-2555_ST25.txt”; Size is 5,000 bytes and it was created on Monday, Apr. 15, 2019 is herein incorporated by reference in its entirety.

### TECHNICAL FIELD

The subject matter disclosed herein is methods and systems for genome-wide screening of presence of genetic elements combined with imaging assays for complex phenotypes to identify relationships between genotypes and phenotypes.

### BACKGROUND

Identifying gene function and impact on disease biology are overarching aims of life science research in the post-genomic era and underpin efforts to understand the meaning of genetic variation in human populations. However, crucial gaps remain in the functional genomics tool set that will slow progress in using genomics to unravel disease biology. Currently, efficient pooled methods for genome-wide screening require either selection of cells based on growth advantage, or physical purification, e.g. by whole-cell fluorescence (using FACS). Many disease processes are characterized by more complex cellular phenotypes including defects in cell or organelle morphology, subcellular localization, cell motility, or gene expression signatures. Other phenotypes of interest may involve transient states (e.g., mitosis), cell-cell interaction, or require dynamic, optical assays (e.g., optogenetic recording of neuronal activity). Image-based, high-content screens using cDNA and RNA interference have uncovered novel genes involved in complex phenotypes, including mitosis, synaptogenesis, and embryogenesis. However, such microplate-based screens are not regularly conducted at the genomic scale due to the expense, labor and automation expertise required. Although “living cell array” screens have reduced some logistical hurdles, they still require individually synthesizing and arraying each gene perturbation reagent.

Pooling gene perturbations greatly improves scalability, but restricts phenotypic readouts, excluding complex and dynamic cellular phenotypes that can be resolved by imaging.

Historically, pooled screens have relied on enriching populations of cells for a phenotype of interest, followed by next-generation sequencing (NGS) to measure changes in perturbation abundance. Common enrichment-based phenotypes include differential cell fitness (e.g., under drug selection) (Shalem et al., 2014; Wang et al., 2014) and differential fluorescence of a marker (e.g., a genetic reporter or immunostained protein), followed by separation of a target population via fluorescence-activated cell sorting (FACS) (Parnas et al., 2015). Although highly scalable, these approaches are limited to population-level measurement, often rely on indirect reporters for the biological activity of interest and necessarily reduce phenotypes to single or a few parameters. Recently, pooled perturbations were integrated first with single cell RNA-seq (Adamson et al., 2016; Datlinger et al., 2017; Dixit et al., 2016; Jaitin et al., 2016) and subsequently with chromatin accessibility profiling by ATAC-seq and protein detection by mass cytometry (Rubin et al., 2018; Wroblewska et al., 2018) as an alternative route to achieve high-dimensional readout for pooled screens. These molecular profiling screens capture a high-dimensional representation of cell state, yielding more information about perturbation effects. However, the scale of single cell profiling assays is still limited by costs, and the relation between RNA or other molecular profiles and higher order cellular phenotypes is often unknown. Moreover, single cell profiling approaches are necessarily destructive, and thus cannot be used to directly monitor dynamic cellular processes.

Imaging offers an attractive alternative as it can collect many spatially and temporally resolved parameters from millions of individual cells. A multitude of optical assays have been optimized for genetic screens of protein localization, enzyme activity, metabolic state, molecular/cellular dynamics and cellular morphology in a variety of biological contexts, including mitosis (Moffat et al., 2006; Neumann et al., 2010), endocytosis (Collinet et al., 2010), viral infection (Karlas et al., 2010), differentiation (Chia et al., 2010), metabolism (Guo et al., 2008), DNA damage (Floyd et al., 2013), autophagy (Orvedahl et al., 2011) and synaptogenesis (Linhoff et al., 2009). However, these and other high-content screens required expensive and laborious testing of arrayed perturbations, while image-based screening of pooled perturbations has thus far only been demonstrated in bacterial systems (Emmanuel Lawson).

### SUMMARY

The invention provides an improved in situ method for screening presence of genetic elements in cells or populations of cells.

In one aspect, the invention provides a method for screening cells for presence of one or more genetic elements comprising: a) culturing a cell or cell population in one or more discrete volumes; b) introducing one or more polynucleotides into the cell or cell population, wherein each polynucleotide comprises nucleic acid sequences encoding a sequence defining one or more optical barcodes and the one or more genetic elements, and wherein a different optical barcode is assigned to each genetic element or a group of the one or more genetic elements, or wherein the genetic element sequence is the optical barcode; c) incubating the cell or cell population to allow for expression of RNA transcripts comprising the one or more optical barcodes; d) detecting genomic, genetic, epigenetic, proteomic, and/or phenotypic differences caused by the one or more genetic elements in the cell or cell population; and e) detecting the optical barcode by an in situ sequencing method to identify the one or more genetic elements present in the cell or cell population. In certain embodiments, the RNA transcripts can be an RNA polymerase I or RNA polymerase III transcripts. In certain embodiments, the one or more genetic elements comprise one or more genetic perturbations.

In an embodiment, the polynucleotide sequence encoding one or more genetic elements comprises or encodes a gene, a modified/damaged/non-natural nucleotide or nucleotide analog, an overexpressed gene, an RNAi based system, a regulatory RNA, a non-coding RNA, an mRNA, a zinc finger nuclease, a transcription activator-like effector nuclease (TALEN), a meganuclease, a computationally designed protein, a computationally designed RNA, or a CRISPR-Cas system. In another embodiment, the polynucleotide sequence encoding one or more genetic elements encodes a CRISPR-Cas system. The CRISPR-Cas system can be a CRISPR-Cas9 or a CRISPR-Cpf1 system. In an embodiment, the polynucleotide sequence encodes one or more guide sequences.

In an embodiments, introducing one or more polynucleotides in the cell or cell population comprises introducing at least two polynucleotides. In embodiments, the one or more cells or the cell population in the one or more discrete volumes, in certain instances, two or more discrete volumes, may comprise the same genotype. In an embodiment, the one or more genetic elements target genes in a pathway or intracellular network. The one or more genetic elements cause gene knock-down, gene knock-out, gene activation, gene insertion, insertion of a foreign sequence tag, or regulatory element deletion. The foreign sequence tag can comprise epitope tag or fluorescent protein tag. In certain embodiments, the one or more genetic elements comprise pooled single or combinatorial CRISPR-Cas-based perturbation with a genome-wide library of sgRNAs.

In an embodiment, detecting genomic, genetic, epigenetic, proteomic, and/or phenotypic differences caused by the one or more genetic elements in the cell or cell population comprises determining a phenotypic difference by capturing a microscopic image or time series of microscopic images of the cell or cell population; and correlate the phenotypic difference to the identified one or more genetic elements. In another embodiment, detecting genomic, genetic, epigenetic, proteomic, and/or phenotypic differences caused by the one or more genetic elements in the cell or cell population comprises measuring differences of DNA, RNA, protein, or post-translational modification, or measuring differences of protein or post translational modification correlated to RNA and/or DNA level(s).

In certain embodiments, the method can further comprise generating a cDNA copy of the RNA transcripts prior to detecting the optical barcode. The method can even further comprise amplifying the generated cDNA copy prior to detecting the optical barcode. The generated cDNA copy can be amplified by rolling circle amplification or hybridization chain reaction.

In certain embodiments, the in situ sequencing method can be selected from the group consisting of fluorescent in situ RNA sequencing (FISSEQ), in situ mRNA-seq, padlock in situ sequencing, sequencing by ligation, SOLiD® sequencing, and sequencing by synthesis.

In an embodiment, the RNA transcripts comprising the one or more optical barcodes can further comprise a cell localization signal or other sequence ultimately localizing the RNA transcripts to a specific location within the cell. The cell localization signal can be a nucleus localization signal or a nuclear export/exclusion signal. In another embodiment, the RNA transcripts can further comprise a premature termination signal to prevent translation of the RNA transcripts comprising the one or more optical barcodes. RNA transcripts may also comprise secondary structure elements (e.g., viral-derived stem-loop motifs) to impede degradation by the cell.

In certain embodiments, the optical barcode is about 4 bp to about 32 bp in length. In certain embodiments, the optical barcode is 12 bp. In certain embodiments, the optical barcode is about 5 bp to about 20 bp in length.

In an embodiment, the one or more polynucleotides are introduced to the cell or cell population by a lentiviral or retroviral system. The lentiviral or retroviral system can have reduced recombination activity, or template switching activity, or multiple integration activity. In an embodiment, the lentiviral or retroviral system can comprise an inhibitor of template switching. In another embodiment, the lentiviral or retroviral system can comprise a carrier polynucleotide. The carrier polynucleotide can comprise non-recombinogenic RNA sequences or proteins that are capable of dimerizing with the polynucleotides comprising optical barcodes and genetic elements. The reduced recombination or template activity can comprise reduced hairpin formation or dimerization through modification, knockdown or knockout of lentiviral or retroviral genomic RNA, or lentiviral or retroviral protein involved in dimerization. The modification, knockdown or knockout of the lentiviral or retroviral genomic RNA or lentiviral or retroviral protein can comprise modification, knockdown or knockout of nucleocapsid (NC)-protein(s) or RNA for expression thereof or modification, knockdown or knockout of stem-loop I element (SLI) element or modification, knockdown or knockout of genomic RNA whereby U5:AUG pairing is prevented, or modification, knockdown or knockout of a dimer initiation site (DIS). In certain embodiments, the lentiviral or retroviral system comprises the genetic element in the 3′ LTR of the lentiviral genome.

The individual discrete volume is a well of a tissue culture plate or a droplet generated on a microfluidic device. The cell or cell population can be contained within or isolated from a tissue sample, for example, a biopsy sample from a mammalian subject or a human subject. The biopsy sample can be a tumor sample. The cell or cell population can be contained within or isolated from a living animal.

In an aspect, the invention provides a system for screening cells for presence of one or more genetic elements, comprising: a) one or more polynucleotides, wherein each polynucleotide comprises nucleic acid sequences encoding a sequence defining one or more optical barcodes and the one or more genetic elements, and wherein a different optical barcode is assigned to each genetic element or a group of the one or more genetic elements, or wherein the genetic element sequence is the optical barcode, and wherein introduction of the one or more polynucleotides into the cell or cell population results in expression of RNA transcripts comprising the one or more optical barcodes; b) a first detection system for detecting genomic, genetic, epigenetic, proteomic and/or phenotypic differences caused by the one or more genetic elements in the cell or cell population; and c) a second detection system for detecting the one or more optical barcodes in the cell or cell population by in situ sequencing.

In an embodiment, the system can further comprise one or more components for generating a cDNA copy of the RNA transcripts prior to detecting the one or more optical barcodes. In another embodiment, the system can further comprise one or more components for amplifying the generated cDNA copy.

In certain embodiments, the one or more polynucleotides are packaged in a lentiviral or retroviral system. The lentiviral or retroviral system can have reduced recombination activity, or template switching activity, or multiple integration activity. In certain embodiments, the lentiviral or retroviral system comprises the genetic element in the 3′ LTR of the lentiviral genome.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 is a diagram illustrating different pooled screening methods.

FIG. 2 provides microscopic images of in situ sequencing screening method.

FIG. 3 is a schematic of the optical barcoding screening method.

FIG. 4 provides cycle-by-cycle microscopic images for the optical barcoding method.

FIG. 5A is a graph showing distribution of in situ reads per cell. FIG. 5B is a graph showing mapping accuracy (left y-axis) and cell fraction (right y-axis) vs. quality threshold. The red dashed line represents oligo synthesis error rate. FIG. 5C is a graph showing how complexity scales with barcode length rate for different amounts of error detection and correction.

FIG. 6 is a diagram depicting in situ RNA barcoding.

FIG. 7 is a diagram showing in situ RNA sequencing preparation steps.

FIG. 8A is an illustration of sequencing by ligation method. FIG. 8B is an illustration of sequencing by synthesis method.

FIG. 9 is a set of graphs depicting base calling performance during an example embodiment of in situ sequencing.

FIG. 10 is a set of graphs showing imaging and base quality across sample.

FIG. 11 is a flow chart depicting an example embodiment of in silico design of error-detecting and error-correcting barcode design (SEQ ID NO:1-4).

FIG. 12A is a graph showing mapping cells to barcodes. FIG. 12B is a graph showing reads per cell.

FIG. 13 is a set of graphs showing reporter for barcode fidelity and CRISPR efficiency.

FIG. 14 provides results for generating a synthetic phenotype using an H2B-HA reporter.

FIG. 15 is a set of graphs showing the relationship between ranked barcode and the fraction FR positive.

FIG. 16 is a schematic diagram showing validation of phenotype-to-genotype with a frameshift reporter in an example embodiment.

FIG. 17 provides the sgRNA, barcode, and lentiviral expression vector cloning design for an example embodiment (SEQ ID NO:5-13).

FIG. 18 is a graph showing the frameshift reporter phenotype in an example embodiment.

FIG. 19A is a scatter plot showing frameshift reporter phenotyping accuracy vs cell count. FIG. 19B is a swarmplot showing sgRNA and barcode pairing validated by in situ and FACS.

FIG. 20 is a set of graphs showing representation of barcodes as cell genomic copies (gDNA) vs. in situ detected barcodes.

FIG. 21 provides a schematic for an example screen for an NFkB nuclear translocation assay.

FIG. 22 provides representative microscopic images and quantitation demonstrating p65-mNeon translocates robustly after TNFa and IL-1b stimulation.

FIG. 23 is diagram showing the 87 gene control set used in the NFkB nuclear translocation assay.

FIG. 24 provides representative microscopic images in the NFkB nuclear translocation screen.

FIG. 25 is a diagram illustrating stimuli and receptors during NFkB nuclear translocation and hits from the 87 gene set.

FIG. 26 provides histograms showing phenotype from the pooled screen.

FIG. 27A is a scatter plot showing signal from TNFa vs. IL-1b stimulation. FIG. 27B is a swarmplot of signal per sgRNA for hit genes grouped by TNFa only, IL-1b only, TNFa and IL-1b.

FIG. 28 is a graph showing average phenotype per sgRNA/barcode.

FIG. 29 provides the percentage of the cells scored positive for part of the 87 gene control set in an example NFkB nuclear translocation assay.

FIG. 30 is a schematic of a two-step library cloning method from oligo pool. The pilot library was made with 72 sgRNA-barcode ultramers and 72 PCR reactions. 192 colonies were verified and 24 plasmids were selected and pooled for virus preparation and infection.

FIG. 31 provides a set of graphs demonstrating that accuracy increases with barcode detection efficiency.

FIG. 32 is a schematic of delivery of barcoded lentiviral plasmid library into target cells.

FIG. 33 is a graph demonstrating infection of lentivirus at low MOI does not remove multiple integrations.

FIG. 34 is a schematic showing altered design for lentiviral expression vectors.

FIGS. 35A-FIG. 35B are schematic showing pooled optical genetic screens. FIG. 35A showing in pooled screens, a library of genetic perturbations is introduced, typically at a single copy per target cell. In existing approaches, cellular phenotypes are evaluated by bulk NGS of enriched cell populations or single-cell gene expression profiling. In pooled optical screens, high-content imaging assays are used to extract rich spatiotemporal information from the sample prior to enzymatic amplification and in situ detection of RNA barcodes, enabling correlative measurement of phenotype and genotype. FIG. 35B showing targeted in situ sequencing is used to read out RNA barcodes expressed from a single genomic integration. Barcode transcripts are fixed in place, reverse transcribed and probed with single-stranded DNA padlock probes, which bind to common sequences flanking the barcode. The 3′ arm of the padlock is extended and ligated, copying the barcode into circularized ssDNA, which is subjected to rolling circle amplification. The barcode sequence is then read out by multiple rounds of in situ sequencing-by-synthesis.

FIGS. 36A-36F are a set of graphs showing identification of perturbation barcodes by in situ sequencing. FIG. 36A is a schematic showing an oligo pool containing perturbation sequences (sgRNAs) and associated 12-nt barcodes is cloned into a lentiviral vector via two rounds of Golden Gate assembly. FIG. 36B is a set of graphs showing expressed barcode sequences are read out by padlock detection, rolling circle amplification and 12 cycles of sequencing-by-synthesis (data shown for lentiGuide-BC-Ef1a) (SEQ ID NOs:14 and 15). FIG. 36C is graph showing per-base quality score over 12 cycles of in situ sequencing, defined as signal for called base divided by signal for all bases. FIG. 36D is a graph showing >80% of barcodes map to 40 designed sequences out of 16.7 million possible 12-nt sequences. FIG. 36E is a graph showing most cells contain multiple barcode reads. FIG. 36F is a graph showing the number of possible barcodes scales geometrically with barcode length. Sufficient 12-nt barcodes can be designed to cover a genome-scale perturbation library while maintaining the ability to detect or correct single base errors (Levenshtein distance d=2 or 3, respectively).

FIGS. 37A-37F are a set of graphs demonstrating accuracy of phenotype-to-genotype mapping assessed with a fluorescent reporter. FIG. 37A is a schematic of a frameshift reporter to convert CRISPR-Cas9-induced indel mutations into a positive fluorescent signal.

FIG. 37B is a set of images showing HeLa-TetR-Cas9 cells were transduced first with the frameshift reporter and subsequently with a library of 1,000 barcodes each encoding a targeting or control sgRNA. FIG. 37C is a set of graphs showing after Cas9 induction, the frameshift reporter is activated and the resulting nuclear-localized HA epitope tag may be stained with labeled antibody and detected in each cell, either by immunofluorescence or via FACS; corresponding barcode sequences may be read out by in situ sequencing or NGS. FIG. 37D is a graph showing targeting and control barcodes are well separated by fraction of HA+ cells.

FIG. 37E is a set of graphs showing that the same cell library was screened by flow sorting cells into HA+ and HA− bins and performing next-generation sequencing of the genomically integrated barcode. FIG. 37F is a graph showing comparison of barcode abundances measured by in situ sequencing or NGS (R2=0.55).

FIGS. 38A-38E are a set of graphs showing a screen for regulators of NF-κB signaling. FIG. 38A is a flowchart showing workflow for CRISPR-Cas9 knockout-based screening using a fluorescently tagged reporter cell line. Screen hits are identified by the failure to translocate p65-mNeon following stimulation with IL1β or TNFα cytokines. FIG. 38B is a graph showing known NF-κB regulators are identified as high-ranking screen hits. Translocation defect is defined as the integrated difference in the distribution of translocation scores (pixelwise correlation between mNeon fluorescence and DAPI nuclear stain) relative to non-targeting control guides across three replicate screens. FIG. 38C is a set of graphs showing distribution of translocation scores of identified NF-κB regulators in response to both cytokines. The shaded area depicts the difference between the translocation score distribution for targeting guides and non-targeting control guides (shown in gray). FIG. 38D is a graph showing NF-κB pathway map (KEGG) color-coded as in FIG. 38B. FIG. 38E is a set of graphs showing top-ranked genes were validated with individual CRISPR-Cas9 knockouts. Histograms show the cumulative distribution of IL1β and TNFα-induced translocation scores for each gene knockout compared to parental cells.

FIG. 39 is a schematic showing workflow for padlock detection and sequencing-by-synthesis. In order to determine the identity of the lentiviral vector integrated in each cell, all cellular RNAs are first fixed in place by formaldehyde treatment. A reverse transcription primer containing locked nucleic acid (LNA) bases is hybridized to the mRNA containing the barcode sequence. Complementary DNA (cDNA) is generated using a reverse transcriptase lacking RNase activity, producing an RNA-DNA hybrid. The cells are then fixed once again (“post-fixed”) with a mixture of formaldehyde and glutaraldehyde to improve cDNA retention. A single reaction mix containing RNAse H, a DNA polymerase lacking strand displacement activity, a DNA ligase, and a padlock DNA oligonucleotide is then added. Digestion of the RNA strand exposes the cDNA bases, allowing the padlock to hybridize to the cDNA at sites flanking the barcode. The DNA polymerase extends the padlock, copying the barcode sequence, but does not strand displace the 5′ annealed padlock arm. Once extended, the padlock is then ligated into a single-stranded DNA circle. During this step, the cDNA is retained in place via hybridization to the RNA strand at the LNA-modified bases within the RT primer, which inhibit RNAse H digestion. Phi29 polymerase is used to perform rolling circle amplification (RCA) of the circularized padlock. The 3′ exonuclease activity of Phi29 polymerase digests the single-stranded portion of the cDNA strand, generating a primer for RCA. The amplified single-stranded DNA product contains tandem repeats of the padlock adaptor sequences and barcode, which can be read out by sequencing-by-synthesis (SBS).

FIG. 40 is a graph showing optimization of padlock detection efficiency and amplification yield. Padlock detection efficiency was increased more than two-fold compared to literature protocols [Ke 2013, Chen 2017] by optimizing the dNTP concentration and polymerase used for the padlock extension-ligation reaction. A striking improvement in detection efficiency was observed when using Stoffel fragment with a dNTP concentration 1000-fold less than previously published [Ke 2013]. As a vendor supplying Stoffel fragment except as a custom order could not be found, the similar, commercially available TaqIT polymerase was also tested, which was found to have equal or superior performance. While optimizing post-fixation conditions for detection efficiency, it was observed that modifying the standard 4% formaldehyde fixative to 3% formaldehyde and 0.1% glutaraldehyde led to a dramatic increase in the yield of overall fluorescence signal from each detected barcode. Presumably the improvement is due to an increase in the efficiency of rolling circle amplification, although no mechanism was identified. The protocol comparison was performed on a single multi-well plate, using HeLa-TetR-Cas9 cells transduced with lentiGuide-BC-EF1a. Each data point represents a technical replicate of the in situ protocol.

FIG. 41 is a set of graphs showing arrayed validation of NF-κB screen hits. Individual CRISPR knockout gene perturbations were cloned and transduced into HeLa-TetR-Cas9-p65-mNeon cells for validation of primary screen hits. Induction of Cas9, stimulation by IL1β and TNFα, and translocation score analysis was performed as in the primary screen. Histograms depict the distribution of translocation scores for each knockout.

FIG. 42A is a graph showing validation screen replicates primary screen rankings for IL1b. FIG. 42B is a graph showing validation screen replicates primary screen rankings for TNFα. For both IL1b and TNFα, primary screen gene rankings correlate well (Spearman's p>0.75) with rankings in validation screen of single-gene CRISPR/Cas9 knockouts. The overall protocol provides a high level of sequence specificity, conferred by hybridization of the RT primer to a unique priming site, hybridization of the padlock to the flanking sites, the preference of the ligase to act only on exactly matched DNA, and SBS of the cell-derived barcode sequence itself.

FIG. 43 is a graph demonstrating validation of CROPseq vector in frameshift reporter cells. A pool of 5 targeting and 5 control sgRNAs were inserted into the CROPseq vector and transduced into HeLa-Cas9 frameshift reporter cells at MOI˜10%. After Cas9 induction and frameshift reporter activation, cells were scored for nuclear-localized HA signal. The sgRNA sequence duplicated in the antibiotic resistance cassette was directly sequenced in situ over 4 cycles. The enrichment among cells mapped to a given sgRNA was defined as the ratio of HA+ cells (19.4% of all cells) to HA− cells. The per-read mapping rate was 94% (exact matches to 10/256 possible 4-nt sgRNA prefixes).

FIGS. 44A-44B are a set of graphs depicting detection of combinatorial perturbations. Multiple perturbations can be delivered via separate lentiviral vectors and detected in the same cell. HeLa-TetR-Cas9 cells were sequentially transduced with lentiGuide-BC-EF1a-Zeo (containing a pool of 40 barcodes) and CROPseq-Puro (containing a pool of 10 sgRNAs). The in situ padlock detection protocol was the same as for individual vectors, except the RT primers and padlock probes targeting each vector were mixed at equal ratios. The first cycle of in situ sequencing used only a sequencing primer targeting lentiGuide-BC. FIG. 44A is a set of images showing subsequent cycles used sequencing primers targeting both constructs. FIG. 44B is a graph showing that the cumulative fraction of cells with at least N reads from each library can be calculated. For example, a total of 35% of all cells imaged had 2 or more reads from each library (heatmap position 2,2).

FIGS. 45A-45F. Identification of perturbation barcodes by in situ sequencing. FIG. 45A Schematic of perturbation detection by in situ sequencing. Barcodes representing perturbations are expressed on a Pol II transcript and enzymatically converted into cDNA and amplified by RCA. RCA products serve as templates for sequencing-by-synthesis, in which the sequence tags are read out by multiple cycles of fluorescent nucleotide incorporation, imaging and dye cleavage. FIG. 45B A 125-nt oligo pool encoding perturbations (sgRNAs) and associated 12-nt barcodes was cloned into a lentiviral vector and delivered into HeLa cells. Expressed barcode sequences were read out by padlock detection, rolling circle amplification, and 12 cycles of sequencing-by-synthesis (data shown for lentiGuide-BC). A linear filter (Laplacian-of-Gaussian, kernel width σ=1 pixel) was applied to sequencing channels to enhance spot-like features. FIG. 45C Per-base quality score over 12 cycles of in situ sequencing, calculated from signal for called base divided by signal for all bases. FIG. 45D >80% of barcodes map to 40 designed sequences out of 16.7 million possible 12-nt sequences. FIG. 45E Most cells contain multiple barcode reads. FIG. 45F The number of possible barcodes scales geometrically with barcode length. Sufficient 12-nt barcodes can be designed to cover a genome-scale perturbation library while maintaining the ability to detect and reject single or double sequencing errors (minimum pairwise Levenshtein distance d=2 or 3, respectively).

FIGS. 46A-46D Accuracy of phenotype-to-genotype mapping assessed with a fluorescent reporter. FIG. 46A Workflow for CRISPR-Cas9 knockout-based screening of a genetically-encoded frameshift reporter. A library of targeting and non-targeting guides were cloned into either lentiGuide-BC or the CROP-seq vector and transduced into cells at low MOI. Cas9 expression generates indels at the frameshift reporter target locus in cells with a targeting guide and leads to expression of a nuclear-localized HA epitope. HA expression was assayed by immunofluorescence and correlated with sgRNAs detected by in situ sequencing, Frameshift reporter accuracy was estimated using the relative abundances of HA+ cells showing targeting and non-targeting guides (A and B, respectively). FIG. 46B Targeting and control barcodes expressed from lentiGuide-BC in HeLa-TetR-Cas9 cells were well separated by fraction of HA+ cells. FIG. 46C The same cell library was screened by flow sorting cells into HA+ and HA− bins and performing next-generation sequencing of the genomically integrated barcode. FIG. 46D The experiment was repeated across a panel of cell lines using the CROP-seq library and an optimized padlock detection protocol, yielding a similar distribution of mapped reads and frameshift reporter accuracies.

FIGS. 47A-47E A screen for regulators of NF-κB signaling. FIG. 47A Workflow for CRISPR-Cas9 knockout-based screening using a fluorescently tagged reporter cell line. Screen hits were identified by the failure of p65-mNeonGreen to translocate following stimulation with IL-1β or TNFα cytokines. FIG. 47B Known NF-κB regulators were identified as high-ranking screen hits. Cells were assigned translocation scores based on the pixelwise correlation between mNeonGreen fluorescence and a DAPI nuclear stain. The translocation defect for a gene was defined based on the integrated difference in the distribution of translocation scores relative to non-targeting control sgRNAs across three replicate screens.

FIG. 47C Cumulative distributions of translocation scores (second-ranked guide) of known NF-κB regulators in response to both cytokines. The shaded areas depict the difference between the translocation score distributions for targeting sgRNAs and non-targeting control sgRNAs (gray). FIG. 47D NF-κB pathway map (KEGG HSA04064) color-coded as in (B). FIG. 47E Top-ranked genes were validated with individual CRISPR-Cas9 knockouts. Histograms show the cumulative distributions of IL-1β and TNFα-induced translocation scores (averaged over two guides) for each gene knockout compared to wildtype cells (gray).

FIG. 48A-48D High-content analysis and a live-cell screen group regulators by impact on morphology and p65 translocation kinetics. FIG. 48A Cells from the primary screen were analyzed based on morphological features. Dimensionality reduction by Principal Components Analysis (PCA) grouped genes by known function in the NF-κB pathway and suggested possible functions for previously uncharacterized genes. Genes are plotted by PCA component 1 (46% of variance explained) and component 2 (24%). FIG. 48B PCA was carried out based on the first (Q1), second (Q2) and third (Q3) quartiles of the per-gene distribution for each morphological parameter. Plotted are the average values for each quartile after standardization across gene categories (color codes same as in (48A)). FIG. 48C The initial 952 gene translocation screen was repeated using live-cell imaging to monitor p65 translocation kinetics (n=361,587 analyzed cells; error bars indicate 95% confidence interval). In addition to positive regulators identified in the initial screen (knockout leads to decreased translocation), the live cell screen found negative regulators (knockout leads to sustained translocation), including the canonical negative regulator TNFAIP3/A20. FIG. 48D Gene knockouts led to diverse changes in translocation kinetics. Hierarchical clustering of the time-dependent translocation difference between each gene and the non-targeting controls grouped together KEGG-annotated regulators, as well as other positive and negative regulators with distinct cytokine-specific kinetic signatures. Genes were included in clustering based on ranking by either mean translocation difference at 45 min post-stimulation (timepoint corresponds to initial screen; top 16 genes shown), or integrated translocation difference between 45 min and 6 hr post-stimulation (top 54 genes shown) (STAR Methods).

FIG. 49A-49B Detection of combinatorial perturbations. FIG. 49A Multiple perturbations can be delivered via separate lentiviral vectors and detected in the same cell. HeLa-TetR-Cas9 cells were sequentially transduced with CROPseq-puro (containing a pool of 95 sgRNAs) and CROPseq-zeo (containing a pool of 95 sgRNAs). The in situ padlock detection protocol was the same as for a single vector library. Images were acquired at 10× magnification and could still most barcode spots per cell (scale bar=50 μm); overlapping barcodes yielded poor quality scores and were discarded (grey spots in “base calls” panels of 49A). FIG. 49B The cumulative fraction of cells with at least N reads of different sgRNAs can be calculated (bottom). For example, a total of 70% of all cells imaged had 2 or more reads (heatmap position 2,2).

FIGS. 50A-50B Optimization of CROP-seq padlock probe length. A set of 84 padlock probes was synthesized with binding sites flanking the sgRNA sequence in the CROP-seq vector. Padlock probe length, 5′ binding sequence (i.e., binding site on sgRNA scaffold), and non-binding sequence content were varied (STAR Methods). Padlocks contained a barcode in the non-binding sequence so they could be pooled and tested in a single in situ reaction, using in situ sequencing to demultiplex the padlock identity. The relative detection efficiency (count) and RCA yield (intensity) were quantified using a dye-labeled hybridization probe complementary to the 3′ binding site, which was common across all padlock probes. The experiment was repeated using 2-color and 4-color SBS to minimize bias attributable to mapping the barcode sequence. Data are shown colored by FIG. 50A padlock length (not including the 20 nt added during the gap-fill step) and FIG. 50B Tm of the padlock 5′ binding arm.

FIGS. 51A-51C Schematic of a technical frameshift reporter. FIG. 51A Schematic of a frameshift reporter that converts CRISPR-Cas9-induced indel mutations into a positive fluorescent signal. FIG. 51B The frameshift reporter was read out by microscopy in HeLa-TetR-Cas9-FR cells in the absence of a targeting sgRNA. A myc epitope tag in the original, unedited frame was stained to confirm expression levels. The reporter was found to have a very low background, with zero false positives observed among >400,000 cells (dashed line indicates threshold for defining HA+ cells). FIG. 51C Flow analysis for the FACS-based frameshift reporter screen. Cells from the HA+ and HA− gates were sorted (left) and sgRNA abundance was compared by NGS. Sorted cells from the HA+(middle) and HA− (right) gates were re-analyzed to verify sorting accuracy. The ratio of HA+ to HA− cells in the re-analyzed populations sets an upper bound for relative sgRNA enrichment of ˜300×.

FIG. 52 Frameshift reporter barcode abundance by in situ sequencing and FACS. Comparison of barcode abundances measured by in situ sequencing or NGS (R2=0.55). The relative abundance of 95% of barcodes was within 5-fold (indicated by dashed lines).

FIG. 53 Comparison of primary screen and validation screen rankings. For both IL-1β and TNFα, primary screen gene rankings correlate well (Spearman's p>0.73) with rankings in validation screen of single-gene CRISPR-Cas9 knockouts. Proteasome subunits are not shown as they exhibited severe negative fitness effects in arrayed validation experiments, likely biasing the surviving cells to those with incomplete protein knockout.

FIG. 54A-54E In situ sequencing details. FIG. 54A Normalized intensity and quality score distributions for all reads that map or fail to map for a LentiGuide-BC library of 40 barcodes (experiment from FIG. 45). FIG. 54B Spectral crosstalk matrix showing how different sequencing channels bleed into each other. FIG. 54C Read-level intensity comparison across cycles. Each point represents the intensity in a given cycle (3-12) on the y-axis relative to the intensity in the same channel in an earlier cycle (1 or 2) on the x-axis. Missing panels are due to the relatively small number of barcodes in the pool (e.g. no C in cycle 7). FIG. 54D Fraction of reads that map (edit distance=0) and nearly map (edit distance >0) to a barcode expected in the 40-plex pool. For all reads with quality >0.1, the distribution approximates what is seen by NGS. FIG. 54E Comparison of NGS and in situ read abundances.

FIGS. 55A-55D FIG. 55A IL-1β and TNFα heatmap; FIG. 55B charts translocation score for TNFAIP3, MAP3K7, RIPK1, MED12, and MED24; FIG. 55C charts relative translocation score at <45 min versus >150 minutes for IL-1β and TNFα; FIG. 55D translocation score for IL-1β and TNFα over time for wild type and MED12 and MED24 knockout.

FIGS. 56A-56B Improved detection and sequencing of transcripts in intact mouse colon. FIG. 56A Schematic of three in situ protocols for RNA amplification by in situ rolling circle amplification (RCA). i) Our standard protocol relies on reverse transcription (RT) in fixed tissue sections. ii) Alternatively, padlock probes can hybridize directly to RNA in fixed tissue sections and be circularized using the SplintR ligase to boost efficiency. iii) For either approach, tissue sections can be embedded in a gel matrix that permits protease digestion of the tissue, improving reagent penetration—here we show digestion for our standard approach only. FIG. 56B Direct RNA detection and gel clearing substantially improve in situ detection efficiency in tissue and permit sequencing of endogenous transcripts compared to standard ISS.

FIGS. 57A-57B. Adaptation of alternative in situ detection methods to barcode sequencing. FIG. 57A Schematic of direct mRNA detection method with SNAIL probes (Wang et al 2018) adapted for sgRNA detection. FIG. 57B Starting with a dataset of ˜30 guides/gene, 20 synthetic sets of 200 genes (red) or the full set (blue) were scored for hybridization specificity by adapting the OligoMiner FISH probe design tool (Beliveau et al. 2018). Guides were deemed passing if binding to any other guide target was <5-fold stronger than correct pairing with an unadjusted p-value of 1e-7. Error bars indicate SD.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011)

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, “engineered association” means a library member portion that comprises an engineered structural and/or functional part, including but not limited to a guide sequence for a CRISPR system, or a tag or identifier such as a unique molecular identifier (UMI) or barcode or other tracking element. The engineered structural or functional part is physically associated with the library member in that it is linked to nucleotides or other chemical parts of the library member. Two or more engineered associations are linked when they are comprised by a single polynucleotide or other monomeric molecule. A library polynucleotide comprising engineered associations may be referred to as a “payload” or “template.”

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to International Patent numbers PCT/US16/22718, filed Mar. 16, 2016. Reference is also made to U.S. provisional application Ser. No. 62/133,821 filed Mar. 16, 2015, 62/133,539 filed Mar. 15, 2015, and 62/627,183 filed Feb. 6, 2018.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein are related to new screening methods for the presence of genetic elements. Example embodiments disclosed herein are directed to a new genetic perturbation and screening method that combines advantages of pooled perturbation with imaging assays for complex phenotypes. Specifically, the method may be used to screen pooled genomic perturbations to identify phenotypes and to identify perturbed genes at the single-cell level. A major advantage offered by this approach is the ability to screen for any cellular phenotype that can be identified by high-resolution microscopy—including live-cell phenotypes, protein localization, or highly multiplexed expression profile and mRNA localization by RNA-FISH—in conjunction with a large array of genetic perturbations applied as a pool in a single test volume. This combines the principal benefits of today's pooled (low cost) and arrayed (high information content) screens with single-cell resolution. The embodiments disclosed herein provide approaches based on in situ sequencing methods and are highly suited to screening in cultured and primary cells, post-mitotic cells, such as neural cells, and tissue sections. The methods disclosed herein can be applied to combinatorial screens where two or more genetic perturbations per cell need to be assessed.

The genetic elements may cause gene knock-down, gene knock-out, gene activation, gene insertion, insertion of a foreign sequence tag, or regulatory element deletion. The genetic elements may comprise genetic perturbations. The genetic perturbations may include a gene knock-in, a gene-knock out, gene activation, gene overexpression, or one or more nucleotide insertions deletions, substitutions, or mutations. The genetic perturbation may be generated using, for example, CRISPR-Cas systems, RNAi (siRNA and shRNA), regulatory RNA, non-coding RNA, TALEN, Zn Finger enzymes, site directed mutagenesis, or other genetic engineering methods known in the art, or a combination thereof. CRISPR-Cas systems may include, but are not limited to, CRISPR/Cas9, CRISPR/Cas12, and CRISPR/Cas13.

A pooled library of transcriptional effectors for introducing one or more genetic perturbations is designed and cloned into a suitable vector. For example, the library may contain a set of plasmids or other suitable delivery vectors with each delivery vector encoding one or more genetic perturbations.

In certain example embodiments, one or more polynucleotides or vectors can encode one or more optical barcodes. In certain example embodiments, each polynucleotide or vector encodes a single optical barcode per vector. The optical barcodes can range from about 4 bp to about 32 bp in length. The optical barcodes can be detected using in situ sequencing methods. The in situ sequencing methods can be fluorescent in situ RNA sequencing (FISSEQ), in situ mRNA-seq, sequencing by ligation, SOLiD® sequencing, and sequencing by synthesis.

The invention is further described in the following examples, which do not limit the scope of the invention described in the claims.

Expression of the polynucleotide or vector will result in expression of an RNA transcript comprising the one or more optical barcodes. The optical barcodes may be constitutively expressed or may be under the control of an inducible promoter. In certain example embodiments, the optical barcode is under the control of a CMV promoter. In certain example embodiments, the nucleic acid encoding the optical barcode may further comprise a premature termination signal to prevent translation of the RNA transcript comprising the optical barcode. In certain example embodiments, the optical barcode may further comprise a localization signal to localize the expressed RNA transcript comprising the optical barcode to a particular cellular location. Cellular localization signals are known in the art and can be selected based on a desired target location for localizing the transcript in the cell. In certain example embodiments, the localization signal is a cellular nucleus localization sequence. In one example embodiment, the nuclear localization signal is a 3′ UTR stem loop, including stem loops from viral transcripts and the IncRNA MALAT1.

In certain embodiments, the genetic element comprising a genetic perturbation is a CRISPR guide sequence. In certain embodiments, the guide sequence is also the optical barcode. In certain embodiments, the CRISPR guide sequence is expressed from an RNA polymerase III promoter and is also expressed as part of an RNA polymerase II promoter (see, e.g., Datlinger, Paul, et al. “Pooled CRISPR screening with single-cell transcriptome readout.” Nature methods 14.3 (2017): 297). In certain example embodiments, the guide sequence is located in the LTR of a lentiviral sequence. In certain embodiments, a polynucleotide or a vector comprises nucleic acid sequences encoding one or more guide sequences acting as both an optical barcode and genetic perturbation.

In certain embodiments, a polynucleotide or a vector comprises nucleic acid sequences encoding both a sequence defining one or more optical barcodes and one or more genetic perturbations. The optical barcodes are delivered to cells or populations of cells together with the genetic perturbations. In other embodiments, a polynucleotide or a vector encoding a unique optical barcode may be delivered to a discrete volume receiving one of the above described genetic perturbations. The polynucleotide or the vector may be delivered to a discrete volume receiving the genetic perturbation prior to, concurrently with, or after the genetic perturbation is introduced. The term discrete volume is defined further below.

In certain example embodiments, the polynucleotides or vectors further encode a site specific nuclease capable of introducing the genetic perturbation into a target sequence within a cell or population of cells. Site specific nucleases include, but are not limited to, e.g., a zinc-finger nuclease (ZFN), a transcription activator-like effector nuclease (TALENs) and/or a CRISPR system comprising a Cas protein and sgRNA. In certain example embodiments, the site-specific nuclease is a Cas9, Cas12, or Cas13 nuclease. In certain example embodiments, the Cas, or functional domain thereof, is fused to a second domain. In certain example embodiments, the second domain is a nickase, a deaminase domain (e.g. a deaminase domain from an ADAR or APOBEC) a transcriptional activator, a transcriptional repressor, a recombinase, a transposase, a DNA or histone methyltransferases, a histone nucleases, or an endonuclease recognizing chromatin remodeling loci, such as CTCF sites at loop anchors. In certain example embodiments, the Cas nuclease may be catalytically inactive, for example to repress target gene expression, or facilitate localization of the second domain to a target loci.

In certain example embodiments, the polynucleotides or vectors encode a Cas nuclease, a short guide RNA (sgRNA) and the optical barcode. In certain example embodiments, the sgRNA and optical barcode may be under the control of the same promoter or a separate promoter. In certain example embodiments, the polynucleotides or vectors may further encode a detectable marker such as GFP to allow tracing of the whole cell body of cells successfully transfected with a polynucleotide or vector.

Any suitable vector for delivering the constructs to a single cell or population of cells may be used. In certain example embodiments, the vector is a viral vector. In another example embodiments, the viral vector is a lentiviral vector.

### Detection of Phenotype and Genotype Using Optical Barcodes

The above described constructs are introduced into a single cell or population of cells. The cells may be cultured cells, primary cells, post-mitotic cells, such as neural cells, and tissue sections. The cell or population of cells to be screened are cultured in separate discrete volumes. In certain example embodiments, a single discrete volume is used. As used herein, a “discrete volume” or “discrete space” may refer to a container, receptacle, or other defined volume or space that can be defined by properties that prevent and/or inhibit migration of molecules, particles and/or cells. For example, a discrete volume or space may be defined by physical properties such as walls of a discrete well, tube, or surface of a droplet which may be impermeable or semipermeable. In certain example embodiments, the discrete volume may be any standard tissue culture container such as a tissue culture plate or flask. In certain example embodiments, the discrete volume may be the wells of a standard microwell plate, such as 6 well, 24 well, 96 well, 384 well, or 1,536 well plate. The microwell plate may be made of any material suitable for imaging of the discrete volumes using the imaging modalities described herein. In certain embodiments, the plate may be glass-bottom plates suitable for imaging of the discrete volumes using the imaging modalities described herein. In certain example embodiments, the discrete volume may be a culture chamber in an array of culture chambers defined on a microfluidic device, or droplet generated on a microfluidic device. In certain example embodiments, a single cell or population of cells may be cultured on individual microscopic slides in culture medium.

One or more genetic perturbations are introduced into the individual cells or cell populations in each discrete volume. As noted above, the genetic perturbation may be introduced prior to, concurrent with, or subsequent to delivery of the one or more polynucleotides or vectors described above. A single optical barcode identifying the particular genetic perturbation introduced into each cell or cell population is delivered to each discrete volume. In certain example embodiments, the one or more polynucleotides or vectors encode both the optical barcode and a site-specific nuclease for introducing the genetic perturbation into the cell or population of cells. Delivery of the one or more genetic perturbations and/or one or more polynucleotides or vectors encoding the one or more optical barcodes may be achieved using standard delivery techniques known in the art.

The individual cells or cell populations are incubated for a time sufficient to allow expression of the RNA transcript encoding the optical barcode. The effect of the genetic perturbation may be assessed in the presence of different conditions or challenges to the cells such as, but not limited to, exposure of the individual cells or cell populations to therapeutic agents, or combinations of therapeutic agents at different concentrations and/or durations of exposure. The individual cell or population of cells may also be exposed to different physical parameters such as, but not limited to, different temperatures, atmospheric pressures, atmospheric CO₂ concentrations, atmospheric O₂ concentrations, changes in pH, changes to the composition of the culture media, such as introduction of different additives at varying concentrations and/or durations of exposure, or a combination of any of the above.

Optical assessment of each discrete volume may be made to determine a phenotype of the individual cells or cell population. Optical assessments may be recorded for later use. In one embodiment, observable cell phenotypes may include, but are not limited to, changes in morphology, motility, and cell death. Optical assessments may also include cell-cell contact such as, but not limited to, antigen presentation and synapsing, and interaction with a patterned substrate such as, but not limited to, patterned extracellular matrix proteins. In certain example embodiments, an additional imaging agent may be delivered to cells. For example, dyes or stains that label certain sub-cellular components such as the nucleus, cytoskeleton, endoplasmic reticulum, mitochondria, or cell wells. In addition, molecule-specific labeling agents such as labeled antibodies or labeled nucleic acids may be used to track changes in localization of certain target molecules. In one embodiment, acellular systems may be assessed using optical assays for protein:protein interactions, quantitation of components of interest, enzymatic activity, and the like.

The method disclosed herein may analyze 10,000 perturbations replicated 1000-fold at the single-cell level, for a total of 10,000,000 single-cell assays in a screen, assuming use of a typically operated research microscope, such as the Opera Phenix (PerkinElmer), which can image up to 108 cells per day. Accordingly, as the scale of imaging increases, the scale of the methods disclosed herein may increase as well.

Next, the expressed optical barcodes are detected. Because a unique optical barcode is assigned to each type of genetic perturbation (or each genetic perturbation is an optical barcode), read-out of the optical barcode allows the observed phenotypes described above to be correlated to a particular genotype. Each discrete volume is imaged with the appropriate imaging technique to detect the optical barcode. For example, if the optical barcodes are detected using binding by fluorescently labeled probes, each discrete volume is imaged using a fluorescent microscope. In another example, if the optical barcodes are detected using in situ sequencing, each discrete volume is imaged using a fluorescent microscope with one or more filters matching the wave length or absorption spectrum or emission spectrum of the color labels of the probes. In another example, if the optically encoded particles are colorimetrically labeled, each discrete volume is imaged using a microscope having one or more filters that match the wave length or absorption spectrum or emission spectrum inherent to each color label. Other detection methods are contemplated that match the optical system used, e.g., those known in the art for detecting quantum dots, dyes, etc.

The optically detectable labels may be a particular size, shape, color, refractive index, or combination thereof. The optically detectable label should comprise a material and be of a size that can be resolvable using light spectroscopy, non-linear optical microscopy, phase contrast microscopy, fluorescence microscopy, including two-photon fluorescence microscopy, Raman spectroscopy, or a combination thereof. In certain example embodiments, the optically encoded particle may be naturally optically encoded, that is the particle is detectable using one of the above detection means without further modification. In certain other example embodiments, the particle material making up the optically detectable label is amenable to modification such that it can be made optically detectable using one of the above detection means, for example, by fluorescently or colorimetrically labeling the optically detectable label.

The optically detectable labels may comprise fluorophores, colloidal metal particles, nanoshells, nanotubes, nanorods, quantum dots, hydrogel particles, microspheres—such as polystyrene beads—liposomes, dendrimers, and metal-liposome particles. The optically detectable labels may be of any shape including, but not limited to, spherical, string-like, or rod-like. In certain example embodiments, the optically detectable labels are spherical in shape. In certain example embodiments, the optically detectable labels may be formed in a series of pre-defined shapes or sizes in order to distinguish the optically encoded particles by shape or size. In certain example embodiments, the optically detectable labels may have a diameter of approximately 50 nm to approximately 500 μm, or a length of approximately 50 nm to 500 μm.

In one example embodiment, the optically detectable label is a hydrogel particle. The hydrogel particle may be made from, for example, covalently cross-linked PEG with thiol-reactive functional groups, or low melting point agarose functionalized with streptavidin or nucleic acid. In certain example embodiments, the hydrogel particle may be approximately 50 nm to approximately 500 μm in size. In certain example embodiments, the hydrogel particle is fluorescently or colorimetrically labeled. In certain example embodiments, the optical label is incorporated within the hydrogel particle. In certain other example embodiments, the optical label is attached to the surface of the hydrogel particle.

In certain example embodiments, the optically detectable labels are quantum dots. In certain other example embodiments, the quantum dots may be incorporated into larger particles, such as those described above. The quantum dots may be made of semiconductor materials identifiable in the art as suitable for forming quantum dots. Exemplary quantum dots are available for purchase, e.g., from Sigma-Aldrich. The quantum dots may range in size from approximately 2 nm to approximately 20 nm.

In certain example embodiments, the optically detectable label is a colloidal metal particle. The colloidal metal material may include water-insoluble metal particles or metallic compounds dispersed in a liquid, a hydrosol, or a metal sol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the A13+, Ru3+, Zn2+, Fe3+, Ni2+ and Ca2+ ions.

In certain example embodiments, the optically detectable particles are dendrimers. The dendrimer may be formed using standard methods known in the art. Exemplary dendrimers are available for purchase, e.g., from Sigma-Aldrich. The dendrimer may range in size from 5 nm to 500 nm, depending on the chosen size and length of, e.g., a central core, an interior dendritic structure (the branches), and an exterior surface with functional surface groups.

In certain example embodiments, the probes are fluorescently labeled FISH probes. The probes used herein may be RNA probes, DNA probes, or hybrid RNA/DNA probes. In certain example embodiments, the FISH probes are amine-conjugated oligos coupled to amine-reactive dyes. The sequential binding is carried out using known permeabilization, hybridizing, stripping, and re-hybridizing methods known in the art. In certain example embodiments, the probes are removed between sequential rounds of FISH by incubating the cell or population of cells in a wash solution comprising 60% formamide at 37° C.

In certain example embodiments, the optical barcode encoded in the expressed RNA transcript is detected by hybridization directly to the expressed RNA transcript. In certain other example embodiments, a cDNA copy of the expressed RNA transcript is generated and detection of the optical barcode is achieved by sequential binding to the cDNA copy of the RNA transcript. In certain example embodiments, the cDNA sample is first amplified prior to detection and detection of the optical barcode is achieved by sequential binding of probes to the resulting amplicons, or ligation to a sequencing primer (see e.g. SOLiD sequencing chemistry). In certain example embodiments, amplification is achieved by rolling circle amplification.

In certain example embodiments, the probes comprising the optically detectable labels are bound directly to the optical barcode. In certain other example embodiments, intermediate probes having all the characteristics of the probes labeled with optically detectable labels are used, except the intermediate probes only bind the unique sequences of the optical barcodes and do not carry the optically detectable label. The intermediate probes may be branched probes, with each branch comprising a binding site for a second probe. A second probe comprising a corresponding optically detectable label for each branched probed is then bound to the intermediate probe to generate a detectable signal.

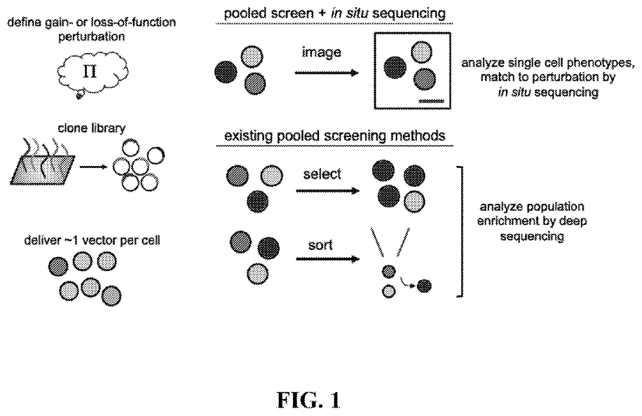

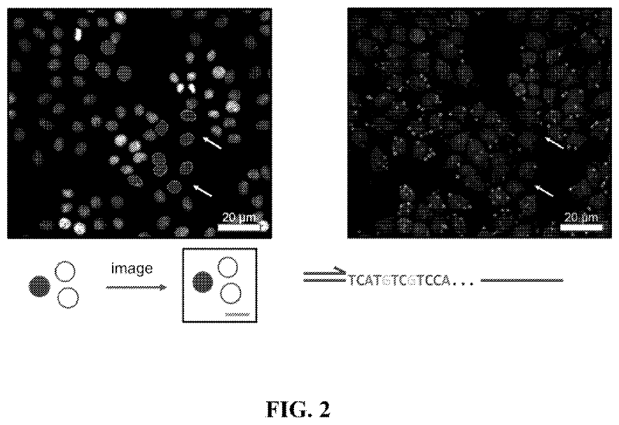

In certain embodiments, the optical barcode is a nucleotide that is about 5 bp to about 20 bp, or about 4 bp to about 32 bp, or about 4 bp to about 50 bp. In certain embodiments, the optical barcode is 8 bp. In certain embodiments, the optical barcode is 12 bp. In certain example embodiments, the optical barcode may be detected directly using an in situ sequencing method. In certain example embodiments, the optical barcode sequence is detected using fluorescent in situ RNA sequencing (FISSEQ), in situ mRNA-seq, padlock in situ sequencing, sequencing by ligation, SOLiD® sequencing, and sequencing by synthesis. In certain example embodiments, the mRNA transcript encoding the optical barcode is sequenced. In certain other example embodiments, a cDNA copy of the mRNA is first generated and then sequenced. Alternatively, the optical barcode may be located in a barcode-specific cDNA primer that can be amplified together with the target. See, for example FIG. 1a of Ke et al. Nature Methods 2013, 10(9)857-60.

### In Situ Sequencing

In certain embodiments, the optical barcode sequence is detected using fluorescent in situ RNA sequencing (FISSEQ) (Lee et al., Nature Protocols 2015, 10(3):442-58). In this method, mRNAs are reverse transcribed in situ using aminoallyl dUTP and adapter sequence-tagged random hexamers. The resulting cDNA fragments are fixed to the cellular protein matrix and circularized. The circular templates are amplified by rolling circle amplification (RCA) followed by sequencing and imaging. This method allows for simultaneous detection of tissue-specific gene expression, RNA splicing, post-transcriptional modifications, and preservation of their spatial information. It is a relatively unbiased method and can achieve transcriptome-wide sampling.

In certain embodiments, the optical barcode sequence is detected using padlock in situ sequencing method (Ke et al. Nature Methods 2013, 10(9)857-60). In this method, after mRNA is reverse transcribed into cDNA, the mRNA is degraded by RNaseH. A padlock probe then binds to the cDNA with a gap between the probe ends over the bases targeted for sequencing. The gap is filled by DNA polymerization and ligated to form a circularized molecule. The circular templates are amplified by RCA followed by sequencing and imaging. Similar to FISSEQ, padlock in situ sequencing allows for preservation of spatial information of analyzed RNA sequences.

In some embodiments, following RCA, the sequencing of the amplified DNA can be achieved using sequencing by ligation or sequencing by synthesis. Sequencing by synthesis relies on a DNA polymerase to incorporate four reversible terminator-bound dNTPs. One base is added per cycle and the fluorescently labeled reversible terminator is imaged as each dNTP is added. Sequencing by ligation uses the mismatch sensitivity of DNA ligase instead to distinguish the sequence of interest and incorporate a pool of fluorescently labeled oligonucleotides of varying lengths. Sequencing by ligation has high accuracy but may encounter problems with palindromic sequences.

In certain embodiments, multiple optical barcode sequences within the same cell can be determined by in situ sequencing method. The optical screening method can also be combined with high-dimensional morphological profiling and in situ multiplexed gene expression analysis. In certain embodiments, perturbing cells can be performed in vivo and phenotypes can be measured within the native spatial context using in situ sequencing of tissue samples.

### Engineered Viral Systems

The non-naturally occurring retroviral systems disclosed herein may comprise a first polynucleotide having at least a first and second engineered association. In an embodiment of the invention, the first engineered association comprises a genetic perturbation. In an embodiment of the invention, both the first and the second engineered association each comprises a genetic perturbation. In an embodiment of the invention, the first engineered association comprises a genetic perturbation and the second engineered association comprises an identifier, such as but not limited to a barcode or a unique molecular identifier.

The systems can comprise a first and second engineered association, but more than two engineered associations are also envisioned. In certain embodiments, the retroviral system may comprise a multiplicity of first polynucleotides. The multiplicity of first polynucleotides may comprise different combinations of engineered associations. As used herein, the term “retroviral” is intended to encompass both retroviral and lentivirus-based systems. The first and second engineered association represent sequences that need to remain associated with one another throughout the life cycle of the polynucleotide. For example, the polynucleotide may be a vector and the first and second association encode elements that need to remain associated on the same polynucleotide for further downstream applications. In certain example embodiments, the first and engineered associations may be located 1 kb or greater apart on the polynucleotide sequence. In certain example embodiments, the engineered associations may be located 2 kb or greater apart on the polynucleotide sequence.

The retroviral system may comprise an inhibitor of recombination or template switching. In certain example embodiment, the retroviral system may further comprise a second polynucleotide. The second polynucleotide may be a carrier polynucleotide comprising non-recombinogenic RNA sequences or sequences with limited homology to the first nucleotide or otherwise configured to impair or prevent homologous recombination with the first polynucleotide when packaged together within a viral particle. In another embodiment, the second polynucleotide may result in reduced hairpin formation or dimerization through modification, knockdown or knockout of retroviral genomic RNA or retroviral proteins involved in dimerization.

In certain example embodiments, the second polynucleotide may be 2 kb to 10 kb in size. In certain example embodiments, the second polynucleotide is 2.0 k, 2.1 kb, 2.2. kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, 4.1 kb, 4.2 kb, 4.3 kb, 4.4 kb, 4.5 kb, 4.6 kb, 4.7 kn, 4.8 kb, 4.9 kb, 5.0 kb, 5.1 kb, 5.2 kb, 5.3 kb, 5.4 kb, 5.5 kb 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, or 10. kb.

In certain example embodiments, the second polynucleotide may be selected to have less than 60%, 59%, 58%, 57%, 56%, 55%, 54%, 53%, 52%, 51%, 50%, 51%, 52%, 53%, 54%, 55%, 56%, 57%, 58%, 56%, 55%, 54%, 53%, 52%, 5′%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% complementarity to the first polynucleotide.

In certain example embodiments, the second polynucleotide is a lentiviral vector. In certain example embodiments, the lentiviral vector has long terminal repeat to long terminal repeat distance (LTR-LTR distance) of 2.5 kb, 2.4 kb, 2.3 kb, 2.2 kb, 2.1 kb, 2.0 kb, 1.9 kb, 1.8 kb, 1.7 kb. 1.6 kb, 1.5 kb, 1.4 kb, 1.3 kb, 1.2 kb. 1.1 kb, or 1.0 kb.

In certain example embodiments, the lentiviral vector comprises one or more LTR mutations in one or both LTR regions that abrogate integration capability.

Other factors that may be considered in selecting or designing second polynucleotide include GC content, presence/absence of repeats, sequence signatures that affect DNA helix parameters, using supercoiled versus relaxed plasmids, nicked or un-nicked plasmids, methylated or non-methylated plasmids.

### Retroviral Systems

The viral backbone of the retroviral system may be any retrovirus suitable for use in delivering expression constructs to cells. Example retroviral systems include Moloney murine leukemia virus (MMULV), feline immunodeficiency virus (Hy), HIV-1 based packaging systems (HIV), and lentiviral based systems. In certain example embodiments, the retroviral system is based on Moloney murine leukemia virus (MoMuLV), Harvey murine sarcoma virus (HaMuSV), murine mammary turmor virus (MuMTV), gibbon ape leukemia virus (GaLV) human immunodeficiency virus (HIV) and Rous Sarcoma Virus (RSV). (see, e.g., Buchscher et al, J. Virol. 66:2731-2739 (1992); Johann et al, J. Virol. 66: 1635-1640 (1992); Sommnerfelt et al, Virol. 176:58-59 (1990); Wilson et al, J. Virol. 63:2374-2378 (1989); Miller et al, J. Virol. 65:2220-2224 (1991); PCT/US94/05700).

Vectors that are based on HIV may retain <5% of the parental genome, and <25% of the genome may be incorporated into packaging constructs, which minimizes the possibility of the generation of revertant replication-competent HIV. The vector region may include sequences form the 5′ and 3′ LTRs of a lentivirus. In some instances, the vector domain includes the R and U5 sequences from the 5′ LTR of a lentivirus and an inactivated or self-inactivating 3′ LTR from a lentivirus. The LTR sequences may be LTR sequences from any lentivirus from any species. For example, they may be LTR sequences from HIV, SIV, FIV or BIV. Where desired, the packaged viral barcoded library may be made up of self-inactivating vectors that contain deletions of the regulatory elements in the downstream long-terminal-repeat sequence, eliminating transcription of the packaging signal that is required for vector mobilization. As such, the vector region may include an inactivated or self-inactivating 3′ LTR. The 3′ LTR may be made self-inactivating by any convenient method. For example, the U3 element of the 3′ LTR may contain a deletion of its enhancer sequence, such as the TATA box, Sp 1 and NF-kappa B sites. As a result of the self-inactivating 3′ LTR, the provirus that is integrated into the host cell genome will comprise an inactivated 5′ LTR. Optionally, the U3 sequence from the lentiviral 5′ LTR may be replaced with a promoter sequence in the viral construct. This may increase the titer of virus recovered from the packaging cell line. An enhancer sequence may also be included. In certain aspects, the viral construct is a non-integrating lentiviral construct, where the construct does not integrate by virtue of having a defective (e.g., by site-specific mutation) or absent integrase gene. Integrase-defective lentiviral vectors are described, e.g., in Banasik and McCray (2010) Gene Therapy 17(2):150-157.

In certain example embodiments, a lentivirus based system is used. Lentiviruses are members of the retrovirus family. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al, J. Virol. 66:2731-2739 (1992); Johann et al, J. Virol. 66: 1635-1640 (1992); Sommnerfelt et al, Virol. 176:58-59 (1990); Wilson et al, J. Virol. 63:2374-2378 (1989); Miller et al, J. Virol. 65:2220-2224 (1991); PCT/US94/05700).

The embodiments disclosed herein may also be useful in non-retroviral based systems, that are pseudo-diploid or otherwise known to have the same recombination and template-switching limitations of lentivirus and retrovirus systems disclosed herein.

Carrier Polynucleotides

The invention provides inhibitors of recombination activity, template switching activity, or multiple integration activity. In an embodiment, the inhibitor of template switching is a carrier polynucleotide. The carrier polynucleotide can be involved in or affect any aspect of lentiviral packaging, and functions to reduce recombination activity or template switching activity, or multiple integration. For example, in an embodiment of the invention, the carrier polynucleotide is packaged with or forms a heterodimer with the polynucleotide comprising the one or more engineered associations, but lacks sufficient homology such that recombination activity, template switching activity, or multiple integration activity is reduced or eliminated. In an embodiment of the invention, the reduction in recombination activity, template switching activity, or multiple integration activity can be 2×, 5×, 10×, 20×, 50×, 100×, 500×, 1000× or greater as compared to packaging without the carrier polynucleotide. In packaging reactions, carrier polynucleotides are usually in excess. In certain embodiments, the carrier polynucleotide to payload polynucleotide ratio in packaging is from 5:1 to 10:1 or from 10:1 to 20:1 or from 20:1 to 50:1, or from 50:1 to 100:1 or from 100:1 to 500:1, of from 500:1 to 1000:1 or greater.

In another embodiment, the inhibitor of recombination activity, or template switching activity, or multiple integration activity can be any carrier polynucleotide transfected into a packaging cell and present with the payload to be packaged, which carrier polynucleotide is not designed to be packaged. Such carriers include, without limitation, single and double stranded DNA, replicable and non-replicable plasmid type vectors, including prokaryotic and eukaryotic vectors. In a non-limiting example set forth herein, bacterial plasmid pUC19, which does not replicate in a packaging cell, is not transcribed, and is not designed to be packaged in a lentiviral particle, is demonstrate to inhibit recombination activity, template switching activity, or multiple integration activity.

In an embodiment, the inhibitor of recombination activity, or template switching activity, or multiple integration activity comprises a polynucleotide designed to hybridize with all or part of the 5′ UTR, including but not limited to such regions as US-PBS complex or the dimer initiation site (DIS). In an embodiment, the inhibitor polynucleotide can be RNA produced concurrently with the payload, or added to the payload prior to packaging. In an embodiment, the inhibitor polynucleotide can be synthetic. Tran et al., 2015, Retrovirology 12:83 reviews conserved determinants of lentiviral genome dimerization.

In an embodiment, recombination activity, template switching activity, or multiple integration activity is reduced by rearranging elements of the payload polynucleotide. This includes without limitation, deletion of 5′ UTR elements and/or introduction of 5′ UTR elements elsewhere in the sequence of the payload to be packaged. In an embodiment, introduction and/or relocation of the DIS provides lentivirus genomes (e.g., payloads) that package predominantly or completely as monomers. Sakuragi et al., 2002, J. Virol. 76:959-967 reports several HIV mutants comprising multiple and rearranged copies of viral E/DLS sequences. According to the invention, 5′ UTR elements can be added and/or rearranged in payload genomes, taking care not to interrupt desired genetic elements (associations) provided therein.

In certain embodiments of the invention, recombination activity, template switching activity, or multiple integration activity is modulated by altering interaction of the payload with the capsid. In one embodiment, the lentivirus nucleocapsid (NC) protein is altered by mutating the zinc-finger region so as to disrupt NC-dependent dimerization. See, e.g., Tran et al., 2015, reviewing 5′ UTR and NC features involved in dimerization.

### Genetic Perturbations

In one example embodiment, the first polynucleotide may encode one or more genetic perturbations. The sequences encoding one or more genetic perturbations may comprise an over-expressed gene, siRNAs, microRNAs, regulatory RNAs, ribozymes, antisense RNAs, guide sequences, or a site-specific nuclease. The sequence encoding one or more genetic perturbations may encode a site-specific nuclease such as, but not limited to, zinc-finger nuclease (ZFN), a transcription activator-like effector nuclease (TALENs), a meganuclease, and/or a CRISPR system. Suitable site-specific nuclease systems are described in further detail below. The perturbation(s) may comprise single-order perturbations. The perturbation(s) may comprise combinatorial perturbations. The perturbations may include gene knock-outs, gene knock-ins, gene overexpression, transpositions, inversions, and/or one or more nucleotide insertions, deletions, or substitutions.

### TALENS

In certain embodiments, the sequence encoding the one or more genetic perturbation encodes a (modified) transcription activator-like effector nuclease (TALEN) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference. By means of further guidance, and without limitation, naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26. The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety. In certain embodiments, targeting is effected by a polynucleic acid binding TALEN fragment. In certain embodiments, the targeting domain comprises or consists of a catalytically inactive TALEN or nucleic acid binding fragment thereof.

### Zn-Finger Nucleases

In certain embodiments, the sequence encoding one or more genetic perturbations comprises or consists of a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference. By means of further guidance, and without limitation, artificial zinc-finger (ZF) technology involves arrays of ZF modules to target new DNA-binding sites in the genome. Each finger module in a ZF array targets three DNA bases. A customized array of individual zinc finger domains is assembled into a ZF protein (ZFP). ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. In certain embodiments, the targeting domain comprises or consists of a nucleic acid binding zinc finger nuclease or a nucleic acid binding fragment thereof. In certain embodiments, the nucleic acid binding (fragment of) a zinc finger nuclease is catalytically inactive.

### Meganuclease

In certain embodiments, the sequences encoding one or more genetic perturbations comprises a (modified) meganuclease, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference. In certain embodiments, targeting is effected by a polynucleic acid binding meganuclease fragment. In certain embodiments, targeting is effected by a polynucleic acid binding catalytically inactive meganuclease (fragment). Accordingly, in particular embodiments, the targeting domain comprises or consists of a nucleic acid binding meganuclease or a nucleic acid binding fragment thereof.

### CRISPR-Cas Systems

In certain embodiments, the sequence encoding the one or more genetic perturbation encodes a (modified) CRISPR/Cas complex or system. General information on CRISPR/Cas Systems, components thereof, and delivery of such components, including methods, materials, delivery vehicles, vectors, particles, and making and using thereof, including as to amounts and formulations, as well as CRISPR/Cas-expressing eukaryotic cells, CRISPR/Cas expressing eukaryotes, such as a mouse, is described herein elsewhere. In certain embodiments, targeting is effected by an oligonucleic acid binding CRISPR protein fragment and/or a gRNA. In certain embodiments, targeting is effected by a nucleic acid binding catalytically inactive CRISPR protein (fragment). Accordingly, in particular embodiments, the targeting domain comprises oligonucleic acid binding CRISPR protein or an oligonucleic acid binding fragment of a CRISPR protein and/or a gRNA.

As used herein, the term “Cas” generally refers to a (modified) effector protein of the CRISPR/Cas system or complex, and can be without limitation a (modified) Cas9, a (modified) Cas12 (e.g. Cas12a “Cpf1”, Cas12b “C2c1,” Cas12c “C2c3”), a (modified) Cas13 (e.g. Cas13a “C2c2”, Cas 13b “Group 29/30”, Cas13c, Cas13d) The term “Cas” may be used herein interchangeably with the terms “CRISPR” protein, “CRISPR/Cas protein”, “CRISPR effector”, “CRISPR/Cas effector”, “CRISPR enzyme”, “CRISPR/Cas enzyme” and the like, unless otherwise apparent, such as by specific and exclusive reference to Cas9. It is to be understood that the term “CRISPR protein” may be used interchangeably with “CRISPR enzyme”, irrespective of whether the CRISPR protein has altered, such as increased or decreased (or no) enzymatic activity, compared to the wild type CRISPR protein. Likewise, as used herein, in certain embodiments, where appropriate and which will be apparent to the skilled person, the term “nuclease” may refer to a modified nuclease wherein catalytic activity has been altered, such as having increased or decreased nuclease activity, or no nuclease activity at all, as well as nickase activity, as well as otherwise modified nuclease as defined herein elsewhere, unless otherwise apparent, such as by specific and exclusive reference to unmodified nuclease.

In some embodiments, the CRISPR effector protein is Cas9, Cas12a, Cas12b, Cas12c, Cas13a, Cas13b, Cas13c, or Cas13d. In some embodiments, the CRISPR effector protein is a DNA-targeting CRISPR effector protein. In some embodiments, the CRISPR effector protein is a Type-II CRISPR effector protein such as Cas9. In some embodiments, the CRISPR effector protein is a Type-V CRISPR effector protein such as Cas12a, Cas12b, or Cas12c. In some embodiments, the CRISPR effector protein is an RNA-targeting CRISPR effector protein. In some embodiments, the CRISPR effector protein is a Type-VI CRISPR effector protein such as Cas13a, Cas13b, Cas13c, or Cas13d.

In some embodiments, the CRISPR effector protein is a Cas9, for instance SaCas9, SpCas9, StCas9, CjCas9 and so forth—any ortholog is envisaged. In some embodiments, the CRISPR effector protein is a Cpf1, for instance AsCpf1, LbCpf1, FnCpf1 and so forth—any ortholog is envisaged. In certain embodiments, the targeting component as described herein according to the invention is a (endo)nuclease or a variant thereof having altered or modified activity (i.e. a modified nuclease, as described herein elsewhere). In certain embodiments, said nuclease is a targeted or site-specific or homing nuclease or a variant thereof having altered or modified activity. In certain embodiments, said nuclease or targeted/site-specific/homing nuclease is, comprises, consists essentially of, or consists of a (modified) CRISPR/Cas system or complex, a (modified) Cas protein, a (modified) zinc finger, a (modified) zinc finger nuclease (ZFN), a (modified) transcription factor-like effector (TALE), a (modified) transcription factor-like effector nuclease (TALEN), or a (modified) meganuclease. In certain embodiments, said (modified) nuclease or targeted/site-specific/homing nuclease is, comprises, consists essentially of, or consists of a (modified) RNA-guided nuclease.

In particular embodiments, more particularly where the nuclease is a CRISPR protein, the targeting domain further comprises a guide molecule which targets a selected nucleic acid. For instance, in the context of the CRISPR/Cas system, the guide RNA is capable of hybridizing with a selected nucleic acid sequence. As used herein, “hybridization” or “hybridizing” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PGR, or the cleavage of a polynucleotide by an enzyme. A sequence capable of hybridizing with a given sequence is referred to as the “complement” of the given sequence

Guide Sequences

In certain example embodiments, one of the engineered associations may comprise one of the above Cas proteins. In another embodiment, one of the engineered associations may comprise a Cas protein and second engineered association may comprise a guide sequence. In yet another embodiment, the engineered associations may comprise two or more guide sequences. As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that an RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoid cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′phosphorothioate (MS), S-constrained ethyl(cEt), or 2′-O-methyl 3′ thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucleotides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells genetically edited by a CRISPR system (see Lee et al., eLife, 2017, 6:e25312, DOI:10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (mePP), 5-methoxyuridine (5moU), inosine, 7-methylguanosine, 2′-O-methyl 3′phosphorothioate (MS), S-constrained ethyl(cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU.

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of the guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complementary stretch (the “anti-repeat” being complementary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas proten (Chen et al. Cell. (2013); 155(7): 1479-1491). In particular embodiments the stem of the stemloop is extended by at least 1, 2, 3, 4, 5 or more complementary basepairs (i.e. corresponding to the addition of 2, 4, 6, 8, 10 or more nucleotides in the guide molecule). In particular embodiments these are located at the end of the stem, adjacent to the loop of the stemloop.

In particular embodiments, the susceptibility of the guide molecule to RNAses or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the complementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously.

In particular embodiment, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends an guide molecule that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm². In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the Cas13 CRISPR-Cas system or complex function. The invention can involve applying the chemical source or energy so as to have the guide function and the Cas13 CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans;4/164/r52), 2. FKBP-FRB based system inducible by rapamycin (or related chemicals based on rapamycin) (see, e.g., www.nature.com/nmeth/journal/v2/n6/full/nmeth763.html), 3. GID1-GAI based system inducible by Gibberellin (GA) (see, e.g., www.nature.com/nchembio/journal/v8/n5/full/nchembio.922.html).

A chemical inducible system can be an estrogen receptor (ER) based system inducible by 4-hydroxytamoxifen (4OHT) (see, e.g., www.pnas.org/content/104/3/1027. abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas13 CRISPR-Cas complex or system, and the binding will induce the change of sub-cellular localization of the polypeptide, leading to the entire polypeptide entering the nucleus of cells. Once inside the nucleus, the guide protein and the other components of the Cas13 CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100 .mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm² to about 100 W/cm². Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm² (FDA recommendation), although energy densities of up to 750 mW/cm² have been used. In physiotherapy, ultrasound is typically used as an energy source in a range up to about 3 to 4 W/cm² (WHO recommendation). In other therapeutic applications, higher intensities of ultrasound may be employed, for example, HIFU at 100 W/cm up to 1 kW/cm² (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm⁻². Even more preferably, the exposure to an ultrasound energy source is at a power density of from about 1 to about 15 Wcm⁻².

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm⁻² to about 10 Wcm⁻² with a frequency ranging from about 0.015 to about 10 MHz (see WO 98/52609). However, alternatives are also possible, for example, exposure to an ultrasound energy source at an acoustic power density of above 100 Wcm⁻², but for reduced periods of time, for example, 1000 Wcm⁻² for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extended length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

In certain example embodiments, the system may comprise a first guide sequence and a second guide sequence such as used in paired nickase system or or self-inactivating systems. Paired nickase systems are used, for example, to minimize off-target effects. Typically, guides are designed in pairs and used with a nickase to introduce two nicks, one on each strand, into a DNA duplex, each nick targeted to adjacent but different sequences of a genomic locus. In an embodiment, the guides are expressed from the same promoter. In and embodiment, the guides are in tandem. In such embodiments, the guides are designed to work together, encoded on a single polynucleotide and packaged together. By reducing or eliminating recombination or template switching activity, the invention improves the performance of multiplexed nickase systems comprising two or more guide pairs (i.e., targeting two or more genetic loci). In a self inactivating (SIN) system two or more loci are targeted. One target comprises, for example, a genomic locus intended to be modified and the second target comprises a locus associated with a CRISPR system component whereby the function of the CRISPR system may be targeted. In certain SIN systems, it will be desired to maintain the linkage of a guide that targets the genomic locus with the guide that targets the CRISPR component. Example self-inactivating systems are disclosed in WO/2015/070083, WO/2015/089354, and WO/2015/089351. Example tandem guide systems are disclosed in WO/2014/204724, WO/2014/093622, and WO/2014/204725.

### Barcodes and Unique Molecular Sequences

In certain example embodiments, one of the engineered associations may be a barcode and/or a unique molecular identifier. The term “barcode” as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment. Although it is not necessary to understand the mechanism of an invention, it is believed that the barcode sequence provides a high-quality individual read of a barcode associated with a single cell, a viral vector, labeling ligand (e.g., an aptamer), protein, shRNA, sgRNA or cDNA such that multiple species can be sequenced together.

Barcoding may be performed based on any of the compositions or methods disclosed in patent publication WO 2014047561 A1, Compositions and methods for labeling of agents, incorporated herein in its entirety. In certain embodiments barcoding uses an error correcting scheme (T. K. Moon, Error Correction Coding: Mathematical Methods and Algorithms (Wiley, New York, ed. 1, 2005)). Not being bound by a theory, amplified sequences from single cells can be sequenced together and resolved based on the barcode associated with each cell.

In preferred embodiments, the engineered association may be a unique molecular identifier (UMI). The term “unique molecular identifiers” (UMI) as used herein refers to a sequencing linker or a subtype of nucleic acid barcode used in a method that uses molecular tags to detect and quantify unique amplified products. A UMI is used to distinguish effects through a single clone from multiple clones. The term “clone” as used herein may refer to a single mRNA or target nucleic acid to be sequenced. The UMI may also be used to determine the number of transcripts that gave rise to an amplified product, or in the case of target barcodes as described herein, the number of binding events. In preferred embodiments, the amplification is by PCR or multiple displacement amplification (MDA).

In certain embodiments, an UMI with a random sequence of between 4 and 20 base pairs is added to a template, which is amplified and sequenced. In preferred embodiments, the UMI is added to the 5′ end of the template. Sequencing allows for high resolution reads, enabling accurate detection of true variants. As used herein, a “true variant” will be present in every amplified product originating from the original clone as identified by aligning all products with a UMI. Each clone amplified will have a different random UMI that will indicate that the amplified product originated from that clone. Background caused by the fidelity of the amplification process can be eliminated because true variants will be present in all amplified products and background representing random error will only be present in single amplification products (See e.g., Islam S. et al., 2014. Nature Methods No:11, 163-166). Not being bound by a theory, the UMI's are designed such that assignment to the original can take place despite up to 4-7 errors during amplification or sequencing. Not being bound by a theory, an UMI may be used to discriminate between true barcode sequences.

Unique molecular identifiers can be used, for example, to normalize samples for variable amplification efficiency. For example, in various embodiments, featuring a solid or semisolid support (for example a hydrogel bead), to which nucleic acid barcodes (for example a plurality of barcodes sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support.

A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. Target molecule and/or target nucleic acids can be labeled with multiple nucleic acid barcodes in combinatorial fashion, such as a nucleic acid barcode concatemer. Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discreet volume-, physical property-, or treatment condition-specific) nucleic acid barcodes. Thus, for example, each member of a set of origin-specific nucleic acid barcodes, or other nucleic acid identifier or connector oligonucleotide, having identical or matched barcode sequences, may be associated with (for example, covalently bound to or a component of the same molecule as) a distinct or different UMI.

The barcode and/or UMI can be attached, or “tagged,” to a target molecule. This attachment can be direct (for example, covalent or noncovalent binding of the nucleic acid identifier to the target molecule) or indirect (for example, via an additional molecule). Such indirect attachments may, for example, include a barcode bound to a specific-binding agent that recognizes a target molecule. In certain embodiments, a barcode is attached to protein G and the target molecule is an antibody or antibody fragment. Attachment of a barcode to target molecules (for example, proteins and other biomolecules) can be performed using standard methods well known in the art. For example, barcodes can be linked via cysteine residues (for example, C-terminal cysteine residues). In other examples, barcodes can be chemically introduced into polypeptides (for example, antibodies) via a variety of functional groups on the polypeptide using appropriate group-specific reagents (see for example www.drmr.com/abcon). In certain embodiments, barcode tagging can occur via a barcode receiving adapter associate with (for example, attached to) a target molecule, as described herein.

Target molecules can be optionally labeled with multiple barcodes in combinatorial fashion (for example, using multiple barcodes bound to one or more specific binding agents that specifically recognizing the target molecule), thus greatly expanding the number of unique identifiers possible within a particular barcode pool. In certain embodiments, barcodes are added to a growing barcode concatemer attached to a target molecule, for example, one at a time. In other embodiments, multiple barcodes are assembled prior to attachment to a target molecule. Compositions and methods for concatemerization of multiple barcodes are described, for example, in International Patent Publication No. WO 2014/047561, which is incorporated herein by reference in its entirety.

In some embodiments, a nucleic acid identifier (for example, a nucleic acid barcode) may be attached to sequences that allow for amplification and sequencing (for example, SBS3 and P5 elements for Illumina sequencing). In certain embodiments, a nucleic acid barcode can further include a hybridization site for a primer (for example, a single-stranded DNA primer) attached to the end of the barcode. For example, an origin-specific barcode may be a nucleic acid including a barcode and a hybridization site for a specific primer. In particular embodiments, a set of origin-specific barcodes includes a unique primer specific barcode made, for example, using a randomized oligo type (SEQ ID NO:16).

A nucleic acid identifier can further include a unique molecular identifier and/or additional barcodes specific to, for example, a common support to which one or more of the nucleic acid identifiers are attached. Thus, a pool of target molecules can be added, for example, to a discrete volume containing multiple solid or semisolid supports (for example, beads) representing distinct treatment conditions (and/or, for example, one or more additional solid or semisolid support can be added to the discreet volume sequentially after introduction of the target molecule pool), such that the precise combination of conditions to which a given target molecule was exposed can be subsequently determined by sequencing the unique molecular identifiers associated with it.

Labeled target molecules and/or target nucleic acids associated origin-specific nucleic acid barcodes (optionally in combination with other nucleic acid barcodes as described herein) can be amplified by methods known in the art, such as polymerase chain reaction (PCR). For example, the nucleic acid barcode can contain universal primer recognition sequences that can be bound by a PCR primer for PCR amplification and subsequent high-throughput sequencing. In certain embodiments, the nucleic acid barcode includes or is linked to sequencing adapters (for example, universal primer recognition sequences) such that the barcode and sequencing adapter elements are both coupled to the target molecule. In particular examples, the sequence of the origin specific barcode is amplified, for example using PCR. In some embodiments, an origin-specific barcode further comprises a sequencing adaptor. In some embodiments, an origin-specific barcode further comprises universal priming sites. A nucleic acid barcode (or a concatemer thereof), a target nucleic acid molecule (for example, a DNA or RNA molecule), a nucleic acid encoding a target peptide or polypeptide, and/or a nucleic acid encoding a specific binding agent may be optionally sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others. In some embodiments, the sequence of labeled target molecules is determined by non-sequencing based methods. For example, variable length probes or primers can be used to distinguish barcodes (for example, origin-specific barcodes) labeling distinct target molecules by, for example, the length of the barcodes, the length of target nucleic acids, or the length of nucleic acids encoding target polypeptides. In other instances, barcodes can include sequences identifying, for example, the type of molecule for a particular target molecule (for example, polypeptide, nucleic acid, small molecule, or lipid). For example, in a pool of labeled target molecules containing multiple types of target molecules, polypeptide target molecules can receive one identifying sequence, while target nucleic acid molecules can receive a different identifying sequence. Such identifying sequences can be used to selectively amplify barcodes labeling particular types of target molecules, for example, by using PCR primers specific to identifying sequences specific to particular types of target molecules. For example, barcodes labeling polypeptide target molecules can be selectively amplified from a pool, thereby retrieving only the barcodes from the polypeptide subset of the target molecule pool.

A nucleic acid barcode can be sequenced, for example, after cleavage, to determine the presence, quantity, or other feature of the target molecule. In certain embodiments, a nucleic acid barcode can be further attached to a further nucleic acid barcode. For example, a nucleic acid barcode can be cleaved from a specific-binding agent after the specific-binding agent binds to a target molecule or a tag (for example, an encoded polypeptide identifier element cleaved from a target molecule), and then the nucleic acid barcode can be ligated to an origin-specific barcode. The resultant nucleic acid barcode concatemer can be pooled with other such concatemers and sequenced. The sequencing reads can be used to identify which target molecules were originally present in which discrete volumes.

### Methods for Making Lentiviral System

In one aspect, the embodiments disclosed herein are directed to method of preparing a lentiviral or retroviral system comprising a polynucleotide having engineered associations comprising a sequence encoding one or more genetic perturbations and a unique molecular sequence wherein the system has reduced recombination or template switching activity. In one embodiment, polynucleotides encoding the one or more genetic perturbation and associated unique molecular sequence are cloned into a suitable lentiviral or retroviral vector (“targeting vector”). Suitable vectors include, for example; pBA571 (Addgene Cat #85968), pMJ114 (Addgene Cat #85995), pMJ179 (Addgene Cat #85996), pMJ117 (Addgene Cat #85997). Carrier plasmids are likewise selected. The carrier plasmids do not include sequences encoding the one or more genetic perturbations or the unique molecular sequence. Instead carrier plasmids are selected to comprise non-recombinogenic sequences, or encode proteins that are capable of dimerizing with the polynucleotide of sequence. Example carrier polynucleotides include pr_H2b-BFB (encoding a histone subunit tagged with blue fluorescent protein) and pLX_TRC131_LacZ (control vector used in ORF screens). In certain embodiments, the carrier plasmid may comprise a lentiviral or retroviral plasmid that has been modified to be non-integrating. For example, a lentiviral vector may be made non-integrating by mutating the 5′ long terminal repeat (LTR) and having a short LTR to LTR distance of 2.1 kb. Example proteins that are capable of dimerizing are disclosed in retroviral nucleoproteins (NC) . . . . The target vector and carrier carrier may then be introduced along with standard lentiviral or retroviral packaging plasmids that encode remaining elements need for full viral particle production into a packaging cell lines to generate a viral clone library, each clone comprising a different target vector and one or more carrier vectors. The target vector may be diluted in a composition with one or more carrier vectors prior to introduction in the packaging cell line. In certain example embodiments, the target vector is diluted in a solution comprising one or more carrier vectors prior to introduction into the packaging cell line at a dilution of 1:10, 1:20, 1:30, 1:40, 1:50, 1:60, 1:70, 1:80, 1:90, 1:100, 1:200, 1:300, 1:400, 1:500, 1:600, 1:700, 1:800, 1:900, 1:1000, 1:2000, 1:3000, 1:4000, or 1:5000.

### Libraries and Cell Lines

The compositions of the present invention further include libraries comprising a multiplicity of the retroviral systems disclosed herein. A number of libraries may be used in accordance with the present invention, including but not limited to, normalized and non-normalized libraries for sense and antisense expression; libraries selected for specific chromosomes or regions of chromosomes (e.g. as comprised in YACs or BACs), which would be possible by inclusion of the f1 origin; and libraries derived from a tissue source; and genomic libraries.

The libraries employed in embodiments of the subject methods can be produced using any convenient protocol. According to certain embodiments, preparing the libraries includes combining polynucleotide having a first engineered association and a second engineered association with a vector construct comprising a vector domain of vector sequence under conditions sufficient to produce transfection plasmids which, upon transfection of a packaging cell, result in the production of viral particles containing the polynucleotide as part of genomic nucleic acids encapsidated in viral protein shells. To prepare the product transfection plasmids used for transfection, a polynucleotide may be inserted into a vector nucleic acid, where any suitable protocol may be employed. Examples of suitable protocols include, but are not limited to: DNA ligase mediated joining, recombination enzyme mediate joining, Gateway® cloning technology (Life Technologies, Carlsbad, Calif.), and the like.

The resultant product transfection plasmids may then be used to transfect a suitable packaging cell line for production of library viral particles. The packaging cell line provides the viral proteins that are required in trans for the packaging of the viral genomic RNA into viral particles. The packaging cell line may be any cell line that is capable of expressing retroviral proteins, including HEK293, HeLa, D17, MDCK, BHK, NIH3T3, CHO, CrFK, and Cf2Th. In some embodiments, the construct is used together with a viral reporter construct which may comprise one or more reporter genes under the control of a constitutive or conditional promoter. The packaging cell line may stably express necessary viral proteins. Such a packaging cell line is described, for example, in U.S. Pat. No. 6,218,181. Alternatively, a packaging cell line may be transiently transfected with plasmids comprising nucleic acids that encode the necessary viral proteins. In another embodiment, a packaging cell line that does not stably express the necessary viral proteins is co-transfected with two or more plasmids. One of the plasmids comprises the viral construct comprising the polynucleotide. The other plasmid(s) comprises nucleic acids encoding the proteins necessary to allow the cells to produce functional virus that is able to infect the desired host cell. The packaging cell line may not express envelope gene products. In this case, the packaging cell line will package the viral genome into particles that lack an envelope protein. As the envelope protein is responsible, in part, for the host range of the viral particles, the viruses preferably are pseudotyped. A “pseudotyped” retrovirus is a retroviral particle having an envelope protein that is from a virus other than the virus from which the RNA genome is derived. The envelope protein may be from a different retrovirus or a non-retrovirus. One envelope protein is the vesicular stomatitis virus G (VSV-G) protein. Thus, the packaging cell line may be transfected with a plasmid that includes sequences encoding a membrane-associated protein, such as VSV-G, that will permit entry of the virus into a target cell. One of skill in the art can choose an appropriate pseudo type specific and/or more efficient for the target cell used. In addition to conferring a specific host range, a chosen pseudotype may permit the virus to be concentrated to a very high titer. Viruses alternatively can be pseudotyped with ecotropic envelope proteins that limit infection to a specific species.

The compositions of the present invention further include retrovirus particles derived from said first and second polynucleotides and other packaging vectors needed to form a complete viral particle. Such retrovirus particles are produced by the transfection of the polynucleotides and/or packaging vectors into retroviral cell packaging cell lines. Thus stably transfected cell lines comprising said sequences are also within the scope of the invention disclosed herein. The compositions of the invention further include provirus sequences derived from the retrovirus particles. The provirus sequences may be present in an integrated form within the genome of a recipient cell, or may be present in a free, circularized form. An integrated provirus is produced upon infection of a recipient cell, wherein the infection leads to the production an integration into the cell genome of the provirus nucleic acid sequence. The circularized provirus sequence may generally be produced upon excision of the integrated provirus from the recipient cell genome.

The compositions of the present invention still further include cells containing the retroviral systems disclosed herein, whether the packaging cell lines or recipient cell lines. Additionally, the present invention includes transgenic animals containing the retroviral systems disclosed herein, including preferably animals containing retroviral systems form which sequences (sense or antisense) are expressed in one or more cells.

### Genetic Screens

In one aspect, the present invention provides for a method of reconstructing a cellular network or circuit, comprising introducing at least 1, 2, 3, 4 or more single-order or combinatorial perturbations to a plurality of cells in a population of cells, wherein each cell in the plurality of the cells receives at least 1 perturbation; measuring comprising: detecting genomic, genetic, proteomic, epigenetic and/or phenotypic differences caused by the perturbations in single cells compared to one or more cells that did not receive any perturbation, and detecting the perturbation(s) in single cells; and determining measured differences relevant to the perturbations by applying a model accounting for co-variates to the measured differences, whereby intercellular and/or intracellular networks or circuits are inferred. The measuring in single cells may comprise single cell sequencing. The single cell sequencing may comprise cell barcodes, whereby the cell-of-origin of each RNA is recorded. The single cell sequencing may comprise unique molecular identifiers (UMI), whereby the capture rate of the measured signals, such as transcript copy number or probe binding events, in a single cell is determined. The model may comprise accounting for the capture rate of measured signals, whether the perturbation actually perturbed the cell (phenotypic impact), the presence of subpopulations of either different cells or cell states, and/or analysis of matched cells without any perturbation.

The single-order or combinatorial perturbations may comprise 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 perturbations. The perturbation(s) may target genes in a pathway or intracellular network.

The measuring may comprise detecting the transcriptome of each of the single cells. The perturbation(s) may comprise one or more genetic perturbation(s). The perturbation(s) may comprise one or more epigenetic or epigenomic perturbation(s). At least one perturbation may be introduced with RNAi- or a CRISPR-Cas system. At least one perturbation may be introduced via a chemical agent, biological agent, an intracellular spatial relationship between two or more cells, an increase or decrease of temperature, addition or subtraction of energy, electromagnetic energy, or ultrasound.

The cell(s) may comprise a cell in a model non-human organism, a model non-human mammal that expresses a Cas protein, a mouse that expresses a Cas protein, a mouse that expresses Cpf1, a cell in vivo or a cell ex vivo or a cell in vitro. The cell(s) may also comprise a human cell. The cell or cell population may be isolated from a tissue sample, for example, a biopsy sample from a mammalian or human subject. The biopsy sample can be a tumor sample.

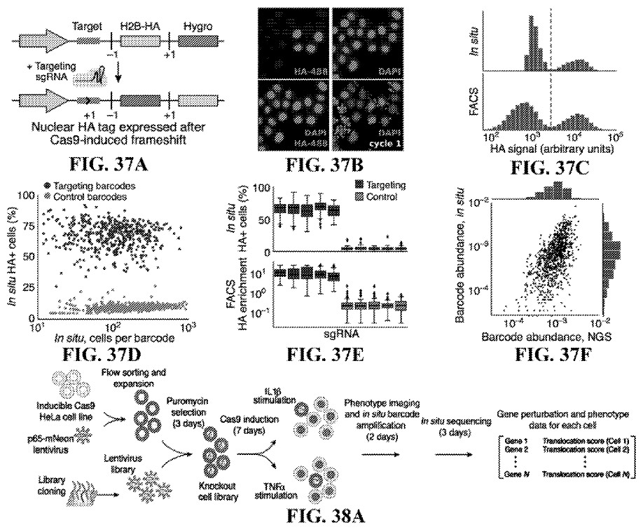

In embodiments, the cells may be engineered to comprise a frameshift reporter target sequence, for example, as detailed in FIG. 37A, FIG. 51A. In embodiments, the engineered cells can be transduced with a pL_FR_Hygro lentiviral vector as described herein, to express an open reading frame consisting of a 50-nt frameshift reporter target sequence, followed by an H2B histone gene with C-terminus HA epitope tag (+1 frameshift), followed by a second H2B gene with C-terminus myc tag (+0 frameshift) and hygro antibiotic resistance gene (+0 frameshift). In embodiments, the genes are preceded by self-cleaving 2A peptides in the same reading frame. In embodiments, the reporter can be activated by co-expression of a Cas protein and targeting guide leading to mutations in the target sequence. In embodiments, the Cas protein is a Type II, Type V or Type VI Cas protein. In one aspect, the Cas protein is a Cas9. The mutations in the target sequence generated by the CRISPR-Cas system may alter the downstream reading frame. In a preferred embodiment, the engineered cell comprises a frameshift of +1 that leads to expression of the H2B-HA protein, which can be visualized by immunofluorescence and detected by microscopy or flow cytometry. In particular embodiments, the engineered cell comprising the frameshift report is in a model non-human organism, e.g. a mouse.

The measuring or measured differences may comprise measuring or measured differences of DNA, RNA, protein or post translational modification; or measuring or measured differences of protein or post translational modification correlated to RNA and/or DNA level(s). The measuring or measured differences may comprise determining a phenotypic difference by capturing a microscopic image of the cell or cell population and correlate the phenotypic difference to the identified one or more genetic perturbations.

The perturbing or perturbation(s) may comprise(s) genetic perturbing. The genetic perturbations may target genes in a pathway or intracellular network. The perturbing or perturbation(s) may comprise(s) single-order perturbations. The perturbing or perturbation(s) may comprise(s) combinatorial perturbations. The perturbing or perturbation(s) may comprise gene knock-down, gene knock-out, gene overexpression, gene activation, gene insertion, or regulatory element deletion. The perturbation may result in a change. The perturbing or perturbation(s) may comprise genome-wide perturbation. The perturbing or perturbation(s) may comprise performing CRISPR-Cas-based perturbation. The perturbing or perturbation(s) may comprise performing pooled single or combinatorial CRISPR-Cas-based perturbation with a genome-wide library of sgRNAs. The perturbations may be of a selected group of targets based on similar pathways or network of targets.

The perturbing or perturbation(s) may comprises performing pooled combinatorial CRISPR-Cas-based perturbation with a genome-wide library of sgRNAs. Each sgRNA may be associated with a unique perturbation barcode. Each sgRNA may be co-delivered with a reporter mRNA comprising the unique perturbation barcode (or sgRNA perturbation barcode).

The perturbing or perturbation(s) may comprise subjecting the cell to an increase or decrease in temperature. The perturbing or perturbation(s) may comprise subjecting the cell to a chemical agent. The perturbing or perturbation(s) may comprise subjecting the cell to a biological agent. The biological agent may be a toll like receptor agonist or cytokine. The perturbing or perturbation(s) may comprise subjecting the cell to a chemical agent, biological agent and/or temperature increase or decrease across a gradient.

The cell may be in a microfluidic system. The cell may be in a droplet. The population of cells may be sequenced by using microfluidics to partition each individual cell into a droplet containing a unique barcode, thus allowing a cell barcode to be introduced.

The perturbing or perturbation(s) may comprise transforming or transducing the cell or a population that includes and from which the cell is isolated with one or more genomic sequence-perturbation constructs that perturbs a genomic sequence in the cell. The sequence-perturbation construct may be a viral vector, preferably a lentivirus vector. The perturbing or perturbation(s) may comprise multiplex transformation or transduction with a plurality of genomic sequence-perturbation constructs.

In another aspect, or in alternative embodiments of aspects described herein, the present invention provides for a method wherein proteins or transcripts expressed in single cells are determined in response to a perturbation, wherein the proteins or transcripts are detected in the single cells by binding of more than one labeling ligand comprising an oligonucleotide tag, and wherein the oligonucleotide tag comprises a unique constituent identifier (UCI) specific for a target protein or transcript. The single cells may be fixed in discrete particles. The discrete particles may be washed and sorted, such that cell barcodes may be added, e.g. sgRNA perturbation barcodes as described above. The oligonucleotide tag and sgRNA perturbation barcode may comprise a universal ligation handle sequence, whereby a unique cell barcode may be generated by split-pool ligation. The labeling ligand may comprise an oligonucleotide label comprising a regulatory sequence configured for amplification by T7 polymerase. The labeling ligands may comprise oligonucleotide sequences configured to hybridize to a transcript specific region. Not being bound by a theory, both proteins and RNAs may be detected after perturbation. The oligonucleotide label may further comprise a photocleavable linker. The oligonucleotide label may further comprise a restriction enzyme site between the labeling ligand and unique constituent identifier (UCI). The ligation handle may comprise a restriction site for producing an overhang complementary with a first index sequence overhang, and wherein the method further comprises digestion with a restriction enzyme. The ligation handle may comprise a nucleotide sequence complementary with a ligation primer sequence and wherein the overhang complementary with a first index sequence overhang is produced by hybridization of the ligation primer to the ligation handle. The method may further comprise quantitating the relative amount of UCI sequence associated with a first cell to the amount of the same UCI sequence associated with a second cell, whereby the relative differences of a cellular constituent between cell(s) are determined. The labeling ligand may comprise an antibody or an antibody fragment. The antibody fragment may be a nanobody, Fab, Fab′, (Fab′)2, Fv, ScFv, diabody, triabody, tetrabody, Bis-scFv, minibody, Fab2, or Fab3 fragment. The labeling ligand may comprise an aptamer. The labeling ligand may be a nucleotide sequence complementary to a target sequence. Single cell sequencing may comprise whole transcriptome amplification. The method in aspects of the invention may comprise comparing an RNA profile of the perturbed cell with any mutations in the cell to also correlate phenotypic or transcriptome profile and genotypic profile.

### Compressed Sensing

Mammalian genomes contain approximately 20,000 genes, and mammalian expression profiles are frequently studied as vectors with 20,000 entries corresponding to the abundance of each gene. It is often assumed that studying phenotypes requires measuring and analyzing these 20,000 dimensional vectors, but some mathematical results show that it is often possible to study high-dimensional data in low dimensional space without losing much of the pertinent information. In one embodiment of the present invention, less than 20,000 genes are perturbed in a population of single cells. Working in low dimensional space offers several advantages with respect to computation, data acquisition and fundamental insights about biological systems.

In one embodiment, perturbations are chosen that are generally part of gene modules or programs, whereby detection of a phenotype allows for the ability to infer phenotype for other perturbations present in a module or gene program.

In alternative embodiments, sparse coding or compressed sensing methods can be used to infer large amounts of data with a limited set of target proteins. In certain embodiments, the phenotype of each of the 20,000 genes can be recovered from random composite measurements. In this regard, reference is made to Cleary et al., “Composite measurements and molecular compressed sensing for highly efficient transcriptomics” posted on Jan. 2, 2017 at biorxiv.org/content/early/2017/01/02/091926, doi.org/10.1101/091926, incorporated herein by reference in its entirety.

In another aspect, or in alternative embodiments of aspects described herein, the present invention provides for a method comprising determining genetic interactions by causing a set of P genetic perturbations in single cells of the population of cells, wherein the method comprises: determining, based upon random sampling, a subset of π genetic perturbations from the set of P genetic perturbations; performing said subset of π genetic perturbations in a population of cells; performing single-cell molecular profiling of the population of genetically perturbed cells; inferring, from the results and based upon the random sampling, single-cell molecular profiles for the set of P genetic perturbations in cells. The method may further comprise: from the results, determining genetic interactions. The method may further comprise: confirming genetic interactions determined with additional genetic manipulations.

The set of P genetic perturbations or said subset of π genetic perturbations may comprise single-order genetic perturbations. The set of P genetic perturbations or said subset of π genetic perturbations may comprise combinatorial genetic perturbations. The genetic perturbation may comprise gene knock-down, gene knock-out, gene activation, gene insertion, or regulatory element deletion. The set of P genetic perturbations or said subset of π genetic perturbations may comprise genome-wide perturbations. The set of P genetic perturbations or said subset of π genetic perturbations may comprise k-order combinations of single genetic perturbations, wherein k is an integer ranging from 2 to 15, and wherein the method comprises determining k-order genetic interactions. The set of P genetic perturbations may comprise combinatorial genetic perturbations, such as k-order combinations of single-order genetic perturbations, wherein k is an integer ranging from 2 to 15, and wherein the method comprises determining j-order genetic interactions, with j<k.

The method in aspects of this invention may comprise performing RNAi- or CRIPSR-Cas-based perturbation. The method may comprise an array-format or pool-format perturbation. The method may comprise pooled single or combinatorial CRISPR-Cas-based perturbation with a genome-wide library of sgRNAs. The method may comprise pooled combinatorial CRISPR-Cas-based perturbation with a genome-wide library of sgRNAs.

The random sampling may comprise matrix completion, tensor completion, compressed sensing, or kernel learning. The random sampling may comprise matrix completion, tensor completion, or compressed sensing, and wherein π is of the order of log P.

The cell may comprise a eukaryotic cell. The eukaryotic cell may comprise a mammalian c ell. The mammalian cell may comprise a human cell. The cell may be from a population comprising 102 to 108 cells and DNA or RNA or protein or post translational modification measurements or variables per cell comprise 50 or more.

The perturbation of the population of cells may be performed in vivo. The perturbation of the population of cells may be performed ex vivo and the population of cells may be adoptively transferred to a subject. The population of cells may comprise tumor cells. The method may comprise a lineage barcode associated with single cells, whereby the lineage or clonality of single cells may be determined.

The perturbing may be across a library of cells to thereby obtain RNA level and/or optionally protein level, whereby cell-to-cell circuit data at genomic or transcript or expression level is determined. The library of cells may comprise or is from a tissue sample. The tissue sample may comprise or is from a biopsy from a mammalian subject. The mammalian subject may comprise a human subject. The biopsy may be from a tumor. The method may further comprise reconstructing cell-to-cell circuits.

The method may comprise measuring open chromatin and may comprise fragmenting chromatin inside isolated intact nuclei from a cell, adding universal primers at cutting sites, and uniquely tagging DNA that originated from the cell.

The method may comprise measuring protein and RNA levels and may comprise CyTOF.

In another aspect, the present invention provides for a method of determining any combination of protein detection, RNA detection, open chromatin detection, protein-protein interactions, protein-RNA interactions, or protein-DNA interactions comprising any of the preceding methods.

In another aspect, the present invention provides for a method for screening compounds or agents capable of modifying a cellular network or circuit comprising performing any method as described herein, wherein perturbing further comprises exposing the cell to each compound or agent.

In another aspect, the present invention provides for a method of identifying a therapeutic, and to a therapeutic identified by the method described herein.

In another aspect, the present invention provides a method of reconstructing a cellular network or circuit, comprising introducing at least 1, 2, 3, 4 or more single-order or combinatorial perturbations to each cell in a population of cells; measuring genomic, genetic and/or phenotypic differences of each cell and coupling combinatorial peturbations with measured differences to infer intercellular and/or intracellular networks or circuits. The single-order or combinatorial perturbations can comprise 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99 or massively parallel perturbations. The perturbation(s) can comprise one or more genetic perturbation. The perturbation(s) can comprise one or more epigenetic or epigenomic perturbation. The perturbation can be introduced with RNAi- or a CRISPR-Cas system. For example, reference is also made to Dahlman et al., Nature Biotechnology (2015) doi:10.1038/nbt.3390 Published online 5 Oct. 2015 to allow efficient orthogonal genetic and epigenetic manipulation. Dahlman et al., Nature Biotechnology (2015) doi:10.1038/nbt.3390 have developed a CRISPR-based method that uses catalytically active Cas9 and distinct single guide (sgRNA) constructs to knock out and activate different genes in the same cell. These sgRNAs, with 14- to 15-bp target sequences and MS2 binding loops, can activate gene expression using an active Streptococcus pyogenes Cas9 nuclease, without inducing double-stranded breaks. Dahlman et al., Nature Biotechnology (2015) doi:10.1038/nbt.3390 use these ‘dead RNAs’ to perform orthogonal gene knockout and transcriptional activation in human cells.

The at least one perturbation can be introduced via a chemical agent, an intracellular spatial relationship between two or more cells, an increase or decrease of temperature, addition or subtraction of energy, electromagnetic energy, or ultrasound. The cell can comprise a cell in a model non-human organism, a model non-human mammal that expresses a Cas protein, a mouse that expresses a Cas protein, a cell in vivo or a cell ex vivo or a cell in vitro. The measuring or measured differences can comprise measuring or measured differences of DNA, RNA, protein or post translational modification; or measuring or measured differences of protein or post translational modification correlated to RNA and/or DNA level(s). The method can include sequencing, and prior to sequencing: perturbing and isolating a single cell with at least one labeling ligand specific for binding at one or more target RNA transcripts, or isolating a single cell with at least one labeling ligand specific for binding at one or more target RNA transcripts and perturbing the cell; and/or lysing the cell under conditions wherein the labeling ligand binds to the target RNA transcript(s).

The method in aspects of this invention may also include, prior to sequencing perturbing and isolating a single cell with at least one labeling ligand specific for binding at one or more target RNA transcripts, or isolating a single cell with at least one labeling ligand specific for binding at one or more target RNA transcripts and perturbing the cell; and lysing the cell under conditions wherein the labeling ligand binds to the target RNA transcript(s). The perturbing and isolating a single cell may be with at least one labeling ligand specific for binding at one or more target RNA transcripts. The isolating a single cell may be with at least one labeling ligand specific for binding at one or more target RNA transcripts and perturbing the cell.

The perturbing of the present invention may involve genetic perturbing, single-order genetic perturbations or combinatorial genetic perturbations. The perturbing may also involve gene knock-down, gene knock-out, gene activation, gene insertion or regulatory element deletion. The perturbation may be genome-wide perturbation. The perturbation may be performed by RNAi- or CRISPR-Cas-based perturbation, performed by pooled single or combinatorial CRISPR-Cas-based perturbation with a genome-wide library of sgRNAs or performing pooled combinatorial CRISPR-Cas-based perturbation with a genome-wide library of sgRNAs.

In addition to loss-of-function (LOF) mutations, embodiments disclosed herein may also be used to modulate transcription without modifying genomic sequences. For example, inactive Cas9 (dCas9) can be catalytically fused to transcriptional activation and repression domains. CRISPR activation (CRISPRa) and CRISPR inhibition (CRISPRi) can be achieved by direct fusion or recruitment of activation and repression domains, such as VP64 and KRAB, respectively. Methods for setting up GOF and LOF genetic screens are described in detail in Joung et al. Nat Protoc. 2017 April: 12(4): 828-863.

Methods and tools for genome-scale screening of perturbations in single cells using CRISPR-Cas9 have been described, herein referred to as perturb-seq (see e.g., Dixit et al., “Perturb-Seq: Dissecting Molecular Circuits with Scalable Single-Cell RNA Profiling of Pooled Genetic Screens” 2016, Cell 167, 1853-1866; Adamson et al., “A Multiplexed Single-Cell CRISPR Screening Platform Enables Systematic Dissection of the Unfolded Protein Response” 2016, Cell 167, 1867-1882; and International publication serial number WO/2017/075294). The present invention is compatible with perturb-seq, such that lentiviral vectors targeting genes for perturbation may be identified and assigned to the proteomic and gene expression readouts of single cells based on transcripts encoding for guide sequence specific barcodes. The present invention can be used to prevent recombination during packaging lentiviral libraries that may shuffle associations between guide sequences and barcode transcripts, thus greatly improving phenotypic readouts associated with a perturbation.

The invention is further described in the following examples, which do not limit the scope of the invention described in the claims.

### EXAMPLES

### Example 1—A Single Lentiviral Vector Integrated in a Cell can be Identified In Situ Via an RNA Barcode

In this Example, it was tested whether existing lentiviral vectors could be modified by adding a barcode that could easily be associated with library elements (e.g., an sgRNA or ORF) and during library cloning and be detectable in situ in a fashion scalable to tens of millions of cells. As pooled assays generally aim to integrate a single lentiviral genome per cell, the barcode from transcribed RNA rather than DNA was detected in order to benefit from the intrinsic amplification of transcription.

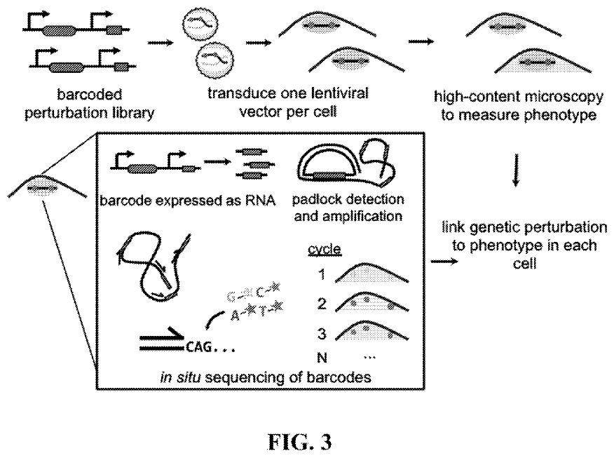

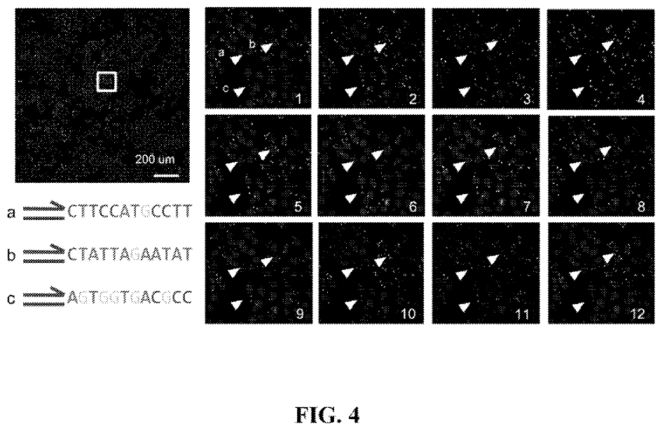

Padlock-based detection of RNA offers excellent specificity due to the requirement to hybridize a transcript-specific primer and padlock, followed by padlock circularization with a highly specific ligase (FIG. 3). A variable sequence in the RNA transcript can be copied into the padlock by binding the padlock to constant regions on either side and extending the 3′ end using a polymerase before ligation. The padlock is then amplified ˜1000-fold by rolling circle amplification. Hybridizing a fluorescently labeled probe yields a bright, compact fluorescent signal (FIG. 4) from each original RNA transcript that was successfully amplified. The variable sequence can be read out in situ by fluorescent sequencing chemistries such as sequencing by ligation and sequencing by synthesis. As a significant drawback of padlock-based RNA detection is low per-transcript sensitivity (1-10%), optimizing both the single-copy lentiviral RNA expression level and the in situ padlock chemistry to maximize the fraction of cells with detected barcodes were focused on initially.

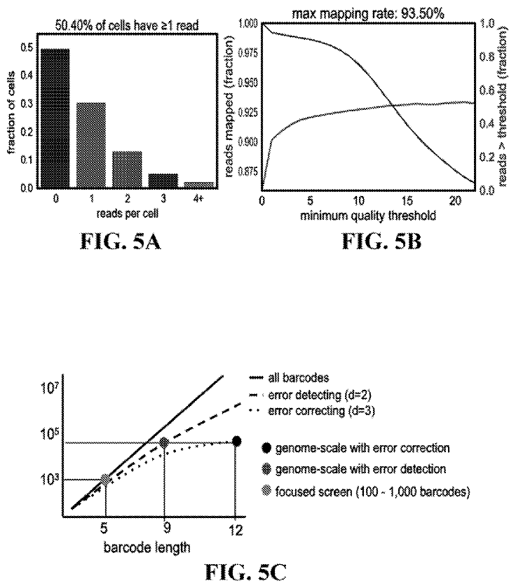

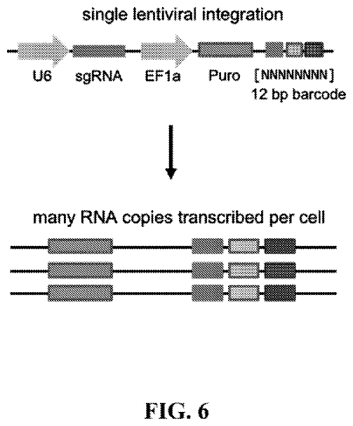

In order to measure the sensitivity of detecting barcodes from a single lentiviral integration, the lentiGuide-Puro vector widely used in CRISPR-Cas9 screening was modified. A 72 bp barcode cassette, including a 12 bp barcode, flanking padlock binding sites, and a downstream reverse-transcription primer binding site, was inserted after the stop codon of the Pol II-driven puro antibiotic resistance gene. This vector (lentiGuide-BC-EF1a) and an alternate vector where the EF1a promoter was replaced with the CMV promoter (lentiGuide-BC-CMV) were transduced into HeLa-TetR-Cas9 cells. The lentiGuide-BC design was selected after testing several alternatives, including direct padlock detection of the Pol III-driven sgRNA, with low-copy lentiviral transduction. Padlock in situ sequencing of cells transduced with lentiGuide-BC-CMV was shown in FIG. 5A. In order to measure barcode swapping between cells due to physical diffusion of RNA or DNA during padlock in situ sequencing, two populations of HeLa-Cas9 cells were labeled with H2B-BFP and H2B-GFP, separately barcoded, and mixed together at an equal ratio. Sequencing results of the mixed population) were shown in FIG. 5B.

In addition to selecting a high-performing barcode cassette, several key steps in the padlock detection protocol were optimized, including use of glutaraldehyde as a post-fixative after reverse transcription and a reduction in dNTP concentration during the gap-fill/ligation step.

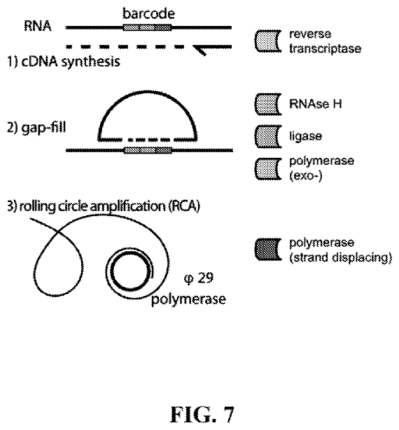

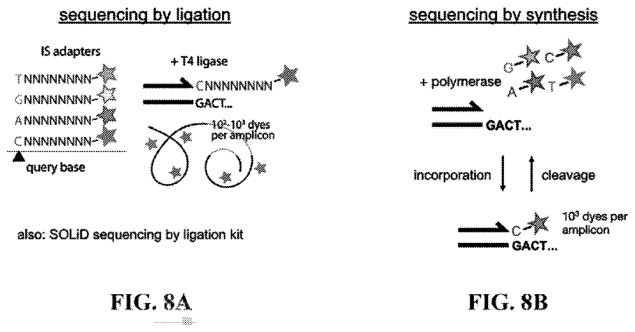

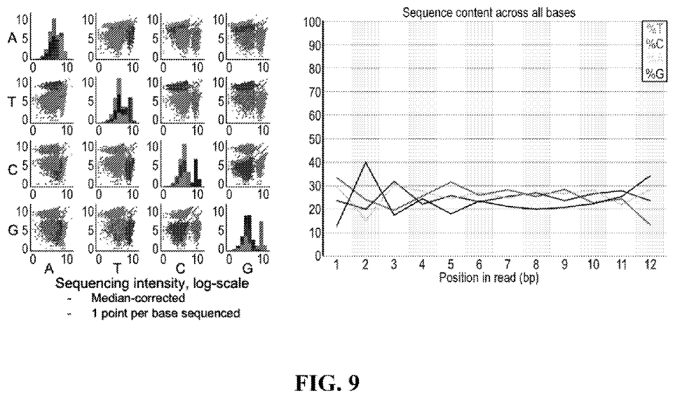

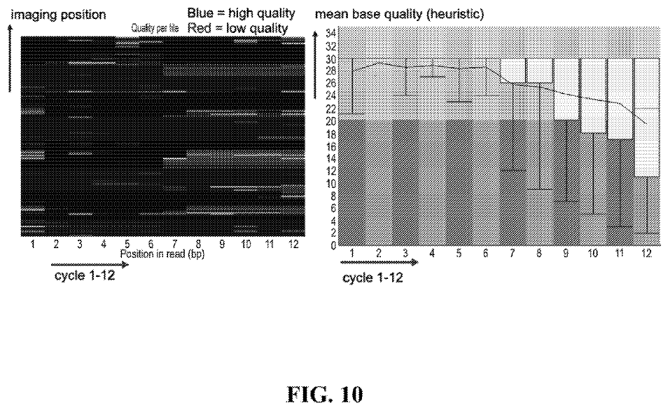

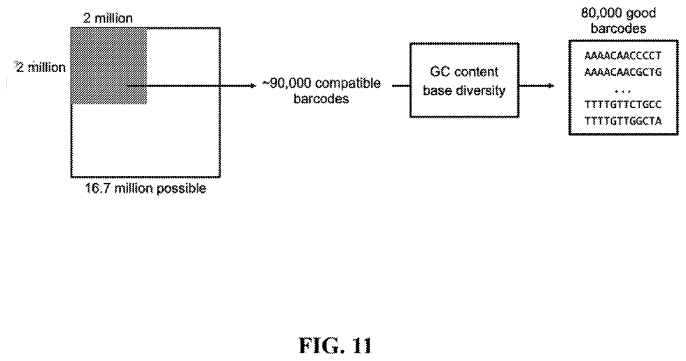

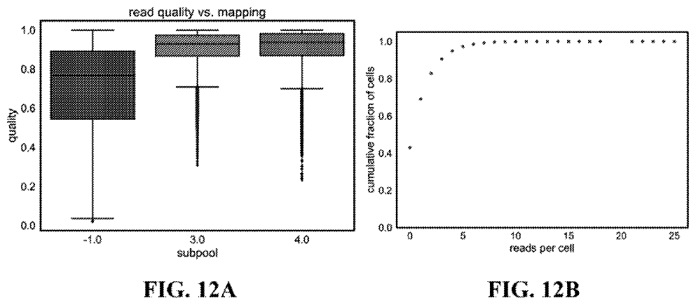

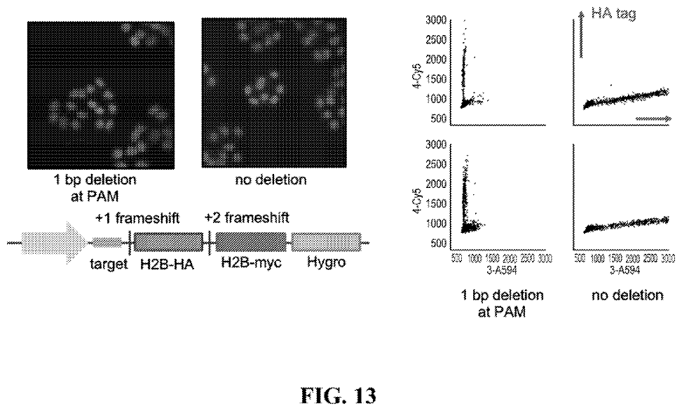

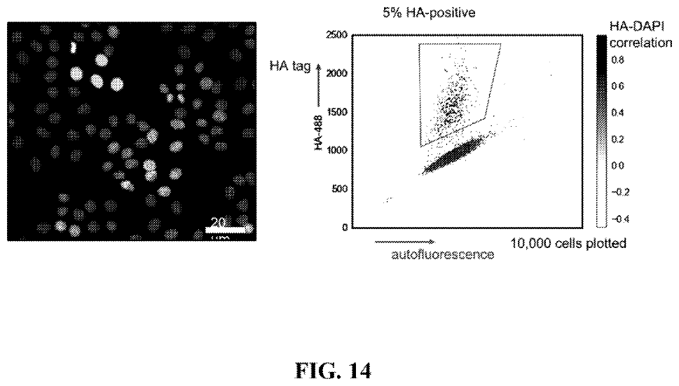

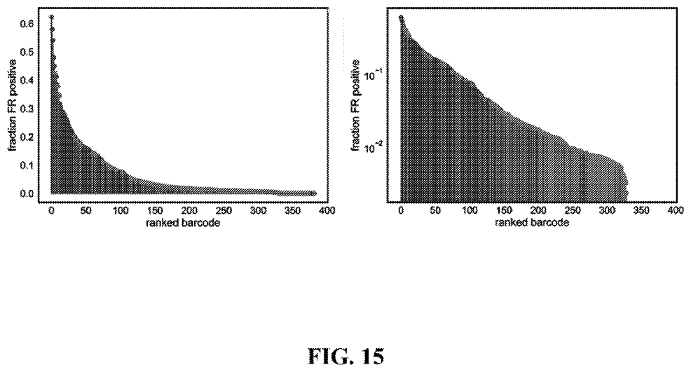

FIG. 7 is a diagram showing in situ RNA sequencing preparation steps. FIG. 8A and FIG. 8B illustrates sequencing by ligation method and sequencing by synthesis method, respectively.

TABLE 1
One combination for sgRNA, bancodes and total experimental time.

| | barcode | cell | | surface area | phenotyping | sequencing | total time* |
|---|---|---|---|---|---|---|---|
| sgRNAs | coverage | coverage | cells | (cm ^{∧}2) | (hours) | cycle (hours) | (workdays) |
| 11 | 7.5 | 3000 | 75,000 | 0.69 | 2 | 1.5 | 4 |
| 500 | 1 | 50 | 50,000 | 2 | 1 | 0 | 4 |
| 500 | 2 | 500 | 1,000,000 | 40 | 11 | 9 | 4 |
| 500 | 1 | 1000 | 3,000,000 | 120 | 33 | 26 | 11 |

TABLE 2
Imaging set up for table 1.

| | | |
|---|---|---|
| | density (cells/cm\hat{ }2) | 25,000 |
| | fraction called at 0.1 error rate | 25% |
| phenotyping | imaging FOV (mm\hat{ }2) | 0.09 |
| | time per slice (s) | 0.5 |
| | z slices | 4 |
| sequencing | imaging FOV (mm\hat{ }2) | 0.36 |
| | time per slice (s) | 2 |
| | z slices | 3 |
| | cycles | 6 |

TABLE 3
Another combination for shRNA, barcode, and total experimental time.

| | barcode | cell | | surface area | phenotyping | sequencing | total time |
|---|---|---|---|---|---|---|---|
| sgRNAs | coverage | coverage | cells | (cm ^{∧}2) | (hours) | cycle (hours) | (days) |
| 25 | 6 | 1000 | 600,000 | 24 | 15 | 11 | 4 |
| 500 | 1 | 50 | 100,000 | 4 | 2 | 2 | 4 |
| 500 | 2 | 300 | 1,200,000 | 48 | 30 | 22 | 8 |
| 500 | 3 | 1000 | 6,000,000 | 240 | 148 | 111 | 37 |

TABLE 4
Imaging set up for table 3.

| | | |
|---|---|---|
| | density (cells/cm\hat{ }2) | 25,000 |
| | fraction called at 0.1 error rate | 50% |
| phenotyping | imaging FOV (mm\hat{ }2) | 0.051 |
| | time per slice (s) | 0.5 |
| | z slices | 1 |
| sequencing | imaging FOV (mm\hat{ }2) | 0.457 |
| | time per slice (s) | 0.2 |
| | z slices | 3 |
| | cycles | 6 |

TABLE 5
Experimental set up for the reverse transcription and RCA steps.

| | | uL | uL | | | uL | uL |
|---|---|---|---|---|---|---|---|
| RT | dilution | (1 rxn) | (MM) | RCA | dilution | (1 rxn) | (MM) |
| RevertAid RT | 5 | 10 | 700 | ph129 | 10 | 5 | 150 |
| buffer (5X) | | | | buffer | | | |
| dNTPs | 40 | 1.25 | 87.5 | dNTPs | 40 | 1.25 | 37.5 |
| (10 mM each) | | | | (10 mM each) | | | |
| BSA (2%) | 100 | 0.5 | 35.0 | BSA (2%) | 100 | 0.5 | 15 |
| RT primer (10 uM) | 10 | 5 | 350.0 | glycerol (50%) | 10 | 5 | 150 |
| RNAseIn (40 U/uL) | 50 | 1 | 70.0 | ph129 (10 U/uL) | 100 | 5 | 150 |
| RevertAid (200 U/uL) | 41.7 | 1.2 | 83.9 | H2O | | 33.3 | 997.5 |
| H2O | | 31.1 | 2173.6 | total volume | | 50 | 1500 |
| total volume | | 50 | 3500 | | | | |

TABLE 6
Experimental set up for the gap-fill and ligation steps.

| gap-fill and ligation | dilution | uL (1 rxn) | uL (MM) |
|---|---|---|---|
| Ampligase buffer (10 X) | 10 | 5 | 50 |
| RnaseH (5 U/uL) | 12.5 | 4 | 40 |
| BSA (2%) | 100 | 0.5 | 5 |
| padlock (2 uM) | 20 | 2.5 | 25 |
| Stoffel fragment (2 U/uL) | 100 | 0.5 | 5 |
| Ampligase (5 U/uL) | 10 | 5 | 50 |
| H2O | | 32.5 | 325 |
| total volume | | 50 | 500 |

### Example 2—Design and Construction of Barcoded CRISPR Perturbation Libraries from Oligo Pools

Robust detection of 12 bp barcodes enabled the construction of CRISPR perturbation libraries with a pre-determined barcode assigned to each sgRNA. From the set of 16.7 million possible 12 bp sequences, 80,000 barcodes with GC content between 25% and 75% were selected, no more than 4 consecutive repeated bases, and minimum substitution and insertion/deletion edit distance of 3 between any pair of barcodes. Ensuring a minimum edit distance is useful for detecting and correcting errors, which arise mainly from DNA synthesis and imaging noise. Individual libraries were amplified from the oligo pool by dial-out PCR and cloned into lentiGuide-BC-CMV via two steps of Golden Gate assembly.

Barcoded perturbation libraries were transduced using a modified lentiviral protocol that reduces barcode swapping due to inter-molecular recombination to and increases the fraction of single integrations at low MOI. To assess the rate of barcode swapping and fraction of single integrations, HeLa-TetR-Cas9 cells were transduced with a lentiGuide-BC-CMV pool at MOI<5%, sorted into plates and expanded. Next-generation sequencing of the sgRNA and barcode from each expanded colony showed that the co-packaging protocol led to a decrease in multiple integrations and recombination compared to standard lentiviral packaging.

### Example 3—Use of a Frameshift Reporter to Validate Phenotype-to-Genotype in Millions of Cells

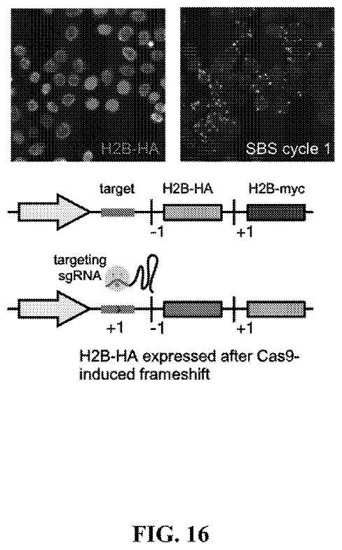

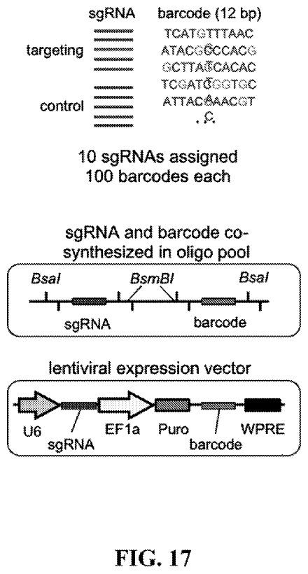

The performance of pooled CRISPR-Cas9 screening using padlock in situ sequencing was benchmarked by targeting a frameshift reporter designed as a highly sensitive and specific readout of Cas9-induced indel mutations (FIGS. 16 and 17). Single-copy integration of lentiGuide-BC carrying a targeting sgRNA activates the reporter in ˜60% of HeLa-TetR-Cas9-FR cells.

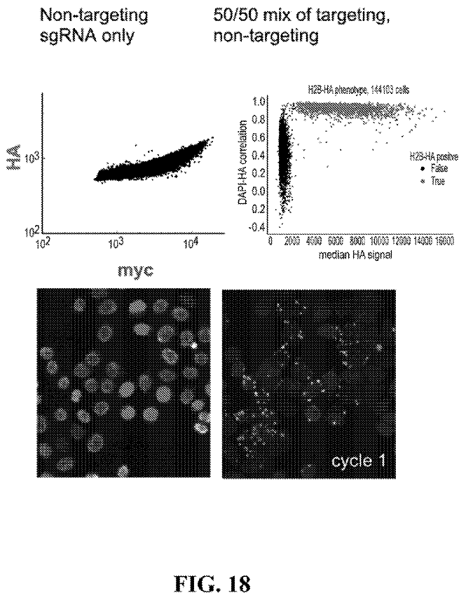

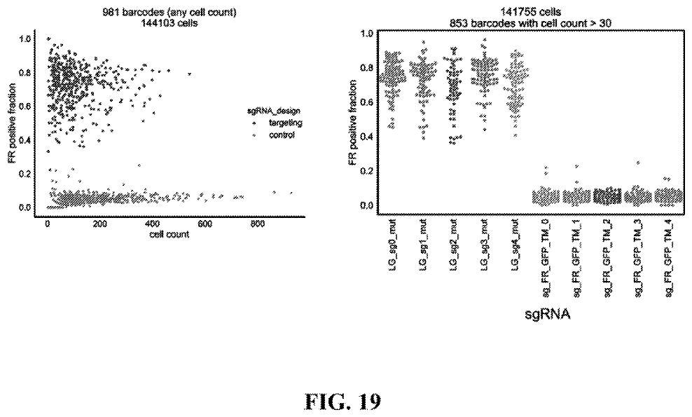

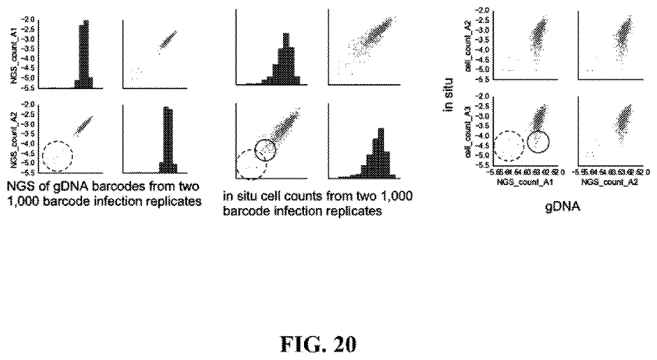

Errors in mapping genotype (targeting or non-targeting sgRNA) to phenotype (reporter activation) could arise from library synthesis and cloning, lentiviral delivery, barcode diffusion during in situ processing, in situ sequencing error, or incorrect assignment of reads to cells during image processing. In order to account for all of these sources of error, HeLa-TetR-Cas9-FR cells were transduced with a lentiGuide-BC library in which 100 barcodes were assigned to each of 5 targeting and 5 non-targeting sgRNAs, for a total of 1,000 barcodes. After antibiotic selection and 7 d of Cas9 induction, cells were fixed, the phenotype of each cell was determined by antibody staining, and the genotype was determined by padlock in situ sequencing. Results of the frameshift reporter were shown in FIGS. 18-20.

### Example 4—Screen for NFkB Nuclear Translocation Assay

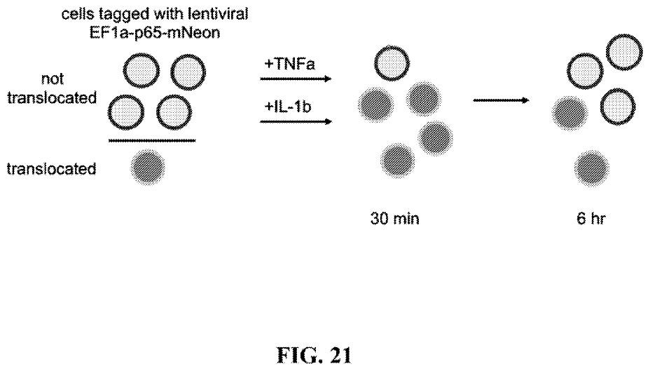

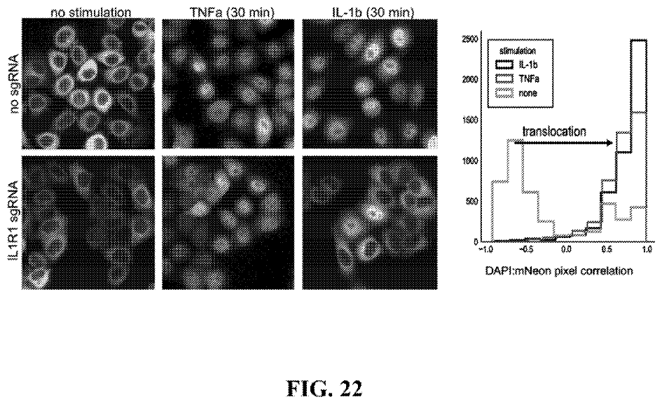

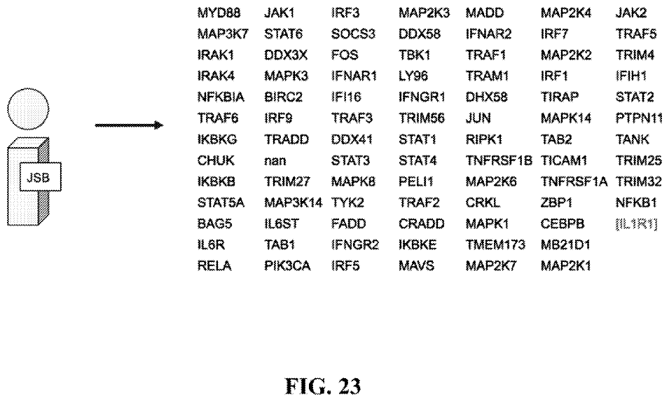

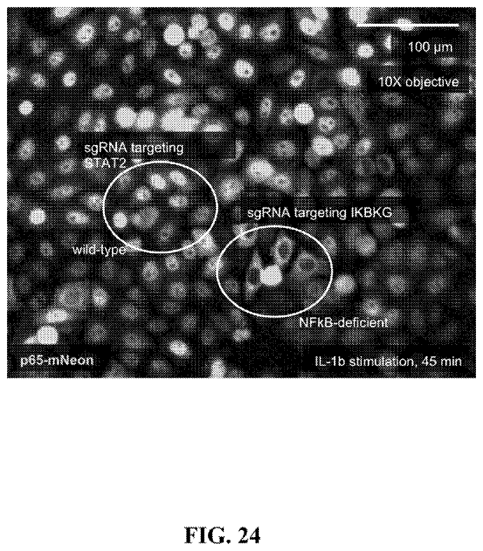

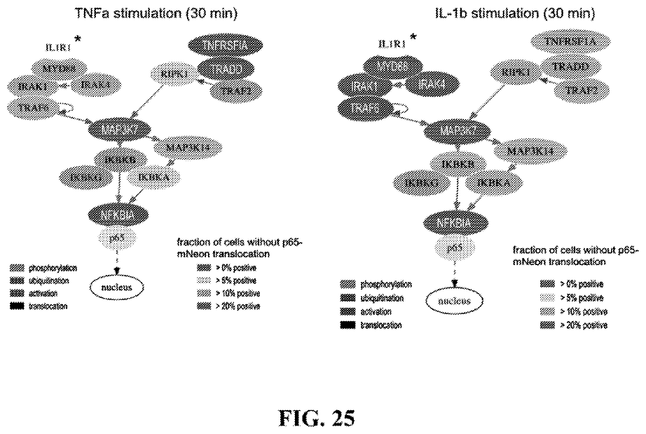

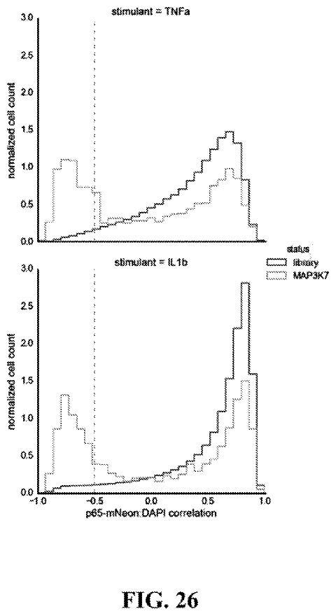

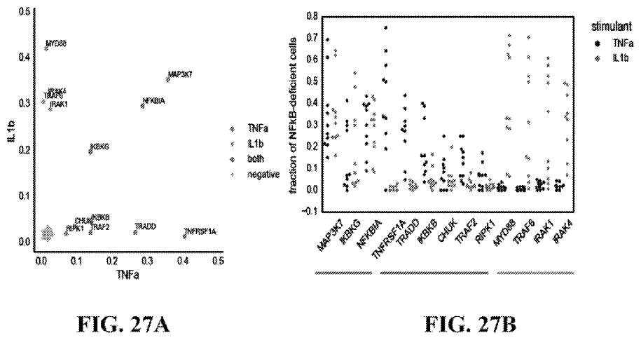

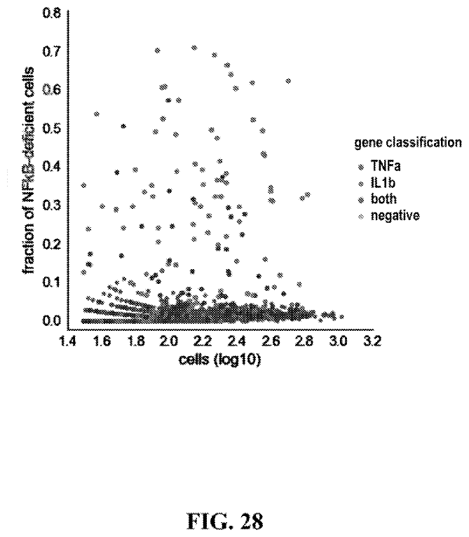

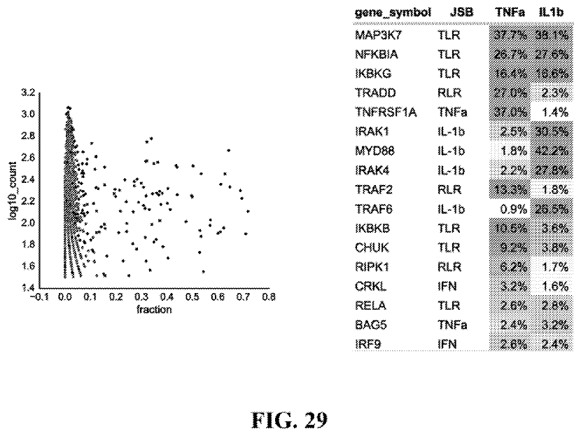

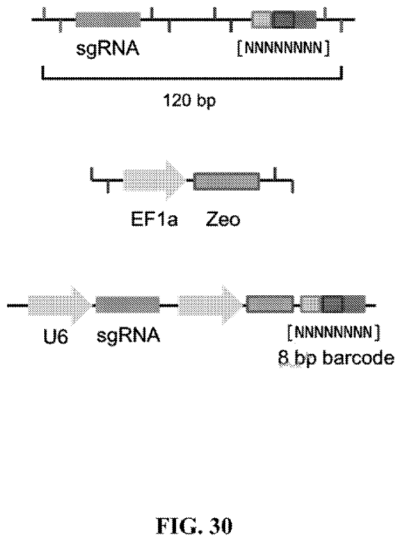

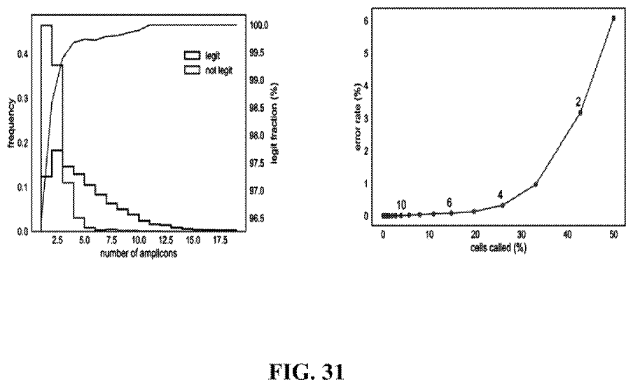

This Examples provides a screen using an NFkB nuclear translocation assay and in situ sequencing method. FIG. 21 and FIG. 22 provide a schematic and some representative microscopic images of the assay, respectively. This assay employs cells tagged with lentiviral EF1a-p65-mNeon construct. Upon TNFa and IL-1b stimulation, translocation of NFkB from the cytoplasm to the nucleus can be scored by fluorescent microscopy. A total of 87 genes were tested in this screen (FIG. 23). FIG. 25 illustrates stimuli and receptors during NFkB nuclear translocation and hits from the 87 gene set. Results of the in situ optical barcoding screen using this assay were shown in FIGS. 26-29.

### Example 5—Debugging: Lentiviral Recombination

Intermolecular provirus recombination during transduction can scramble barcode identities in pooled lentivirus preparations. To test lentivirus integration during in situ cell screening, a pool of 25 plasmids were packaged into lentiviruses using Joung 2017 lentivirus protocol. 500K cells were infected with 0.5 μl virus, and less than 3% cells survived. 192 single cells were sorted by flow cytometry and expanded. sgRNAs and barcodes were PCR amplified separately and deep sequenced. Together, there were 40 correct single integrations, 20 recombined single integrations, and 26 multiple integrations.

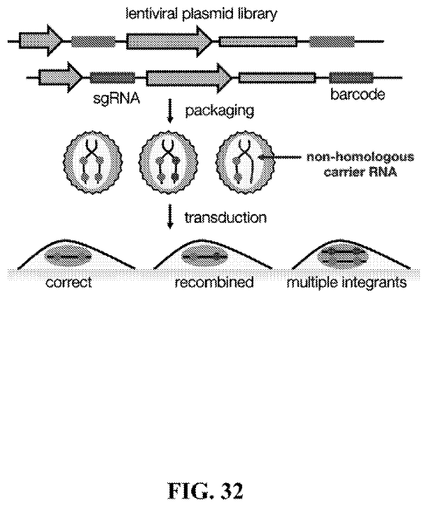

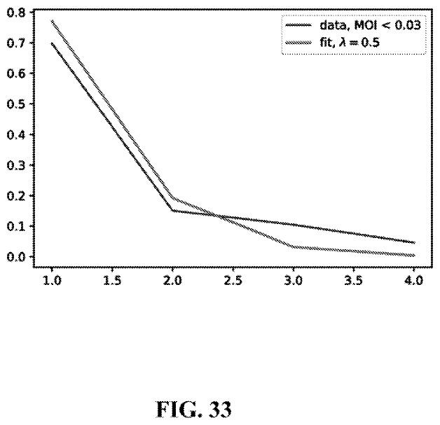

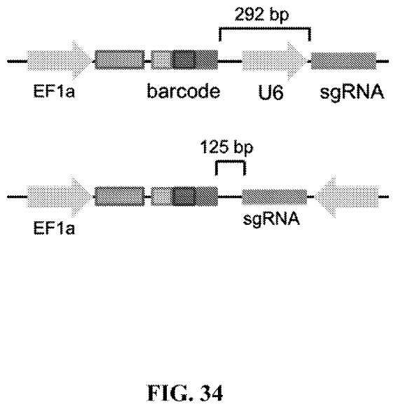

FIG. 32 is a schematic of delivery of barcoded lentiviral plasmid library into target cells. Viral genomes containing sgRNAs and transcribed RNA barcodes, driven by U6 and EF1a promoters, are packaged as dimers into virions and integrated into target cells. Packaging may yield homodimeric or heterodimeric virions or, in the case of co-packaging with a non-homologous carrier lentivirus, a functionally monomeric virion. Virions with two different library elements have the capacity for recombination between sgRNAs and barcodes as well as potential for integration of multiple perturbations into the target cell, whereas co-packaging with a non-homologous vector limits these alternatives. FIG. 33 demonstrates that infecting the lentivirus at low MOI does not remove multiple integrations.

TABLE 7
Different lentivirus conditions tested.

| | pool- | | | | rel- |
|---|---|---|---|---|---|
| | ing | | | | ative |
| condition | step | variation | identity | count | titer |
| 1, mix of pL42.2, | cells | — | correct | 39 | — |
| 42.4, 42.5 GFP+ | | | | | |
| 1, mix of pL42.2, | cells | — | recombined | 0 | — |
| 42.4, 42.5 GFP+ | | | | | |
| 8, individually | virus | — | correct | 45 | 1 |
| packaged | | | | | |
| 8, individually | virus | — | recombined | 0 | 1 |
| packaged | | | | | |
| 10, pL43.pool8.F | plasmid | — | correct | 34 | 1 |
| 10, pL43.pool8.F | plasmid | — | recombined | 17 | 1 |
| 9, pL43.pool8.F_ETP | plasmid | collect | correct | 26 | 1/30 |
| 11 h (250 uL) | | 11 h post- | | | |
| | | transfection | | | |
| 9, pL43.pool8.F_ETP | plasmid | collect | recombined | 17 | 1/30 |
| 11 h (250 uL) | | 11 h post- | | | |
| | | transfection | | | |
| 13, pL43.pool8.F + | plasmid | 1:1000 | correct | 33 | 1/100 |
| pUC19 1:1000 | | dilution | | | |
| | | in pUC19 | | | |
| 13, pL43.pool8.F + | plasmid | 1:1000 | recombined | 2 | 1/100 |
| pUC19 1:1000 | | dilution | | | |
| | | in pUC19 | | | |
| 16, pL43.pool8.F + | plasmid | 1:1000 | correct | 59 | 1/100 |
| SV40-H2B-BFP | | dilution in | | | |
| 1:1000 | | SV40-H2B- | | | |
| | | BFP lenti | | | |
| 16, pL43.pool8.F + | plasmid | 1:1000 | recombined | 0 | 1/100 |
| SV40-H2B-BFP | | dilution in | | | |
| 1:1000 | | SV40-H2B- | | | |
| | | BFP lenti | | | |

### Example 6—Pooled Optical Screens in Human Cells

Large-scale screens play a key role in the systematic discovery of genes underlying cellular phenotypes. Pooling of genetic perturbations greatly increases screening throughput, but has so far limited screens to enrichments defined by cell fitness and flow cytometry, or to comparatively low throughput single-cell gene expression profiles. Although microscopy is a rich source of spatial and temporal information about mammalian cells, high-content imaging screens have been restricted to inefficient arrayed formats. Here, an optical method is introduced to identify perturbations and their phenotypic outcomes at the single-cell level in a pooled setting. Barcoded perturbations are read out by targeted in situ sequencing in a highly accurate and scalable way, which can follow immunofluorescence or live-cell imaging. The technology is used to screen a focused set of 960 genes across >3 million cells for involvement in NF-κB activation by imaging the translocation of RelA (p65), recovering 22 known pathway components and 4 novel candidate positive regulators of TNFα and IL1β-stimulated immune responses.

Forward genetic screens are a powerful tool for finding genes that cause functional phenotypes. A variety of methods exist to disrupt genes, introduce exogenous genes, and modulate gene expression in mammalian cells. Many such perturbations can be pooled together and efficiently quantified by next-generation sequencing (NGS) of cell populations. The phenotypic effect of a perturbation can then be defined as an enrichment in cells carrying the perturbation under different conditions1,2.

Examples of enrichment-based phenotypes that are compatible with pooled screens include differential cell fitness (e.g., under drug selection) and differential fluorescence of a marker assessed by fluorescence-activated cell sorting (FACS) (e.g. a genetic reporter or immunostained protein)3. Although such screens have led to important biological discoveries, many complex phenotypes cannot be physically enriched at scale to provide a sample for NGS analysis. As a result, screens of more complex phenotypes have historically been limited to expensive and laborious testing of individual perturbations in arrayed formats. Recently, single cell genomic profiling was integrated with pooled CRISPR perturbations4-7 as a compelling alternative. However, many other complex phenotypes, especially in cell biology, require imaging, to identify cellular structures, live events and other features, which cannot be captured by any such assays and have not been compatible with pooled formats.

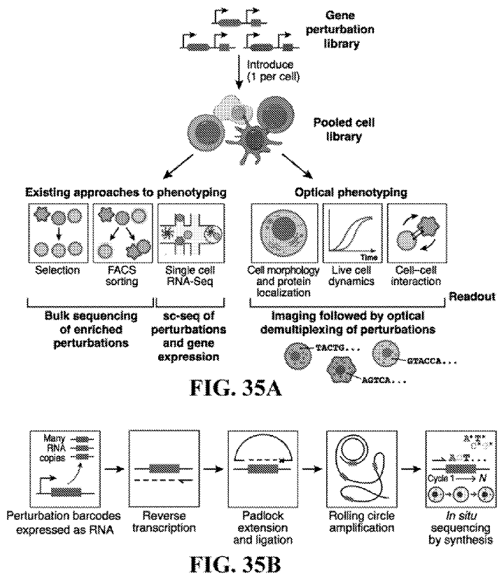

To address this challenge, pooled genetic screens compatible with a wide range of spatially and temporally resolved optical assays were developed in mammalian cells, greatly expanding the variety of phenotypes amenable to high-throughput forward genetics. The approach is to determine both the phenotype and the perturbation identity in each cell by microscopy (FIG. 35A). Each perturbation is encoded by an RNA barcode, and targeted in situ sequencing8 is used to detect which barcode is present in each cell. This strategy uses enzymatic amplification and sequencing-by-synthesis (FIG. 35B) to provide exceptionally high signal and information density compared to alternative methods such as fluorescence in situ hybridization (FISH)9. The method adapts the existing molecular biology pipeline for genetic screens, permitting information-rich phenotyping of thousands of perturbations across millions of cells.

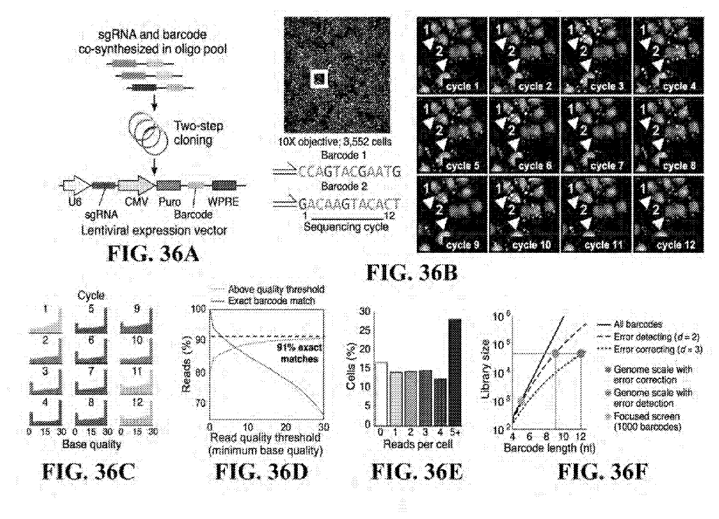

The identity of genetic perturbations was encoded by inserting CRISPR guide RNAs (sgRNAs) and associated 12-nt barcodes into lentiGuide-Puro, a widely-used sgRNA expression vector (FIG. 36A). The barcodes and flanking sequences were inserted into the 3′ UTR of the Pol II-driven antibiotic resistance gene, a highly expressed mRNA suitable for in situ detection. A pooled cloning approach was used where sgRNAs and barcodes are synthesized in tandem on an oligo array, such that sgRNA-barcode pairings are pre-determined (FIG. 36A). As reported for other lentiviral libraries using paired sequences, swapping of barcodes and associated sgRNAs was observed initially in lentiGuide-BC cells due to reverse transcription-mediated recombination11-14. To address recombination, a modified lentiviral packaging protocol was used that reduces barcode swapping from >28% to <5%15.

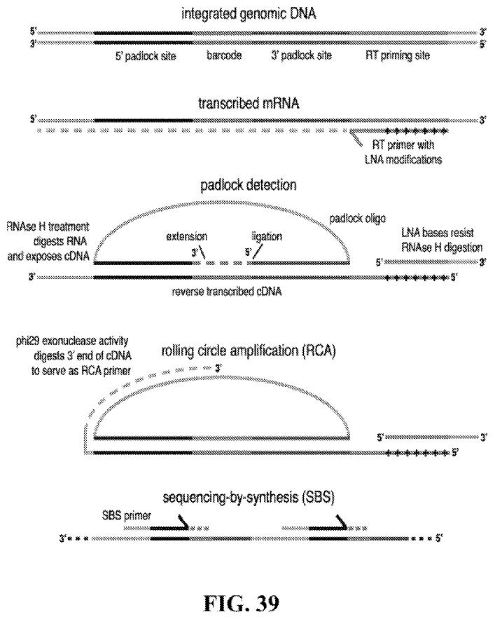

To test in situ detection of perturbations, a library of 40 sgRNA-barcode pairs was transduced into HeLa-TetR-Cas9 cells at low multiplicity of infection (MOI). A reverse transcription primer and padlock oligo complementary to constant regions flanking the barcode were designed, and the barcode was amplified via in situ reverse transcription, padlock extension/ligation, and rolling circle amplification (RCA) (FIG. 35B, FIG. 39). Sequencing in situ using a 4-color sequencing-by-synthesis (SBS) chemistry yielded bright, compact fluorescent spots that retained base specificity over 12 cycles (FIG. 36B, 36C). Image segmentation and base calling analysis produced sequencing reads that exactly matched the set of known barcodes with >85% accuracy (FIG. 36D).

For optical screening to be scalable, the in situ readout step should be able to process millions of cells over a few days, ensuring high coverage per perturbation (typically 100-1,000 cells). High cell throughput was achieved by using low-magnification optics to image a large field of view, which is possible due to the high fluorescence signal-to-noise following padlock-based amplification (FIG. 36B). Specifically, following optimization of key steps in the barcode amplification protocol, particularly the post-fixation and padlock extension/ligation conditions, to maximize detection efficiency and RCA yield, in situ sequencing reads were readily visible at 10× magnification, with one or more exactly mapped reads detected in over 82% of cells transduced with lentiGuide-BC-Ef1a (FIG. 36B, 36E).

Following the initial robust detection of 12-nt barcodes, a set of 83,000 error-correcting barcodes used in subsequent libraries were then designed. As there are 16.7 million possible 12-nt sequences, barcodes with minimum pairwise edit distance of 3 were selected, which provides redundancy to correct single insertion/deletion/substitutions arising from oligo synthesis or in situ processing (FIG. 36F)10.

It was next sought to evaluate the overall accuracy of mapping perturbation genotype to cell phenotype in situ. Errors could arise from oligo synthesis, library cloning, lentiviral delivery, barcode diffusion during in situ processing, barcode readout by in situ sequencing, or incorrect assignment of reads to cells during image processing. To assess the impact of these error sources in the context of CRISPR-Cas9 screening, a lentiviral reporter was built that produces HA-tagged, nuclear-localized H2B protein after a Cas9-induced +1 frameshift in a target region (FIG. 37A). Cells expressing the reporter can be screened either in situ or by FACS. The reporter is highly specific and sensitive, with a mean in situ activation across 5 targeting sgRNAs of 65%+/−5% (65%+/−5% by FACS) and background of <0.01% in the absence of an sgRNA (1%+/−0.3% by FACS) (FIG. 37B, 37C). Cells stably expressing the frameshift reporter were transduced with a lentiGuide-BC library containing 972 barcodes redundantly encoding 5 targeting and 5 control sgRNAs. Cas9 expression was then induced, reporter activation was measured by immunofluorescence and barcode sequences were determined by in situ SBS. All barcodes encoding targeting sgRNAs were separated from control sgRNAs by HA-positive fraction, with a per-cell misidentification rate of approximately 10% (FIG. 37D, 37E). The same cell library was screened via FACS followed by deep sequencing and observed a similar enrichment of targeting sgRNAs (FIG. 37E), with similar performance of most barcodes in both contexts (95% within 5-fold abundance, FIG. 37F). Comparably robust mapping of CRISPR sgRNAs to the frameshift reporter phenotype with the CROPseq vector was achieved, in which the sgRNA sequence is duplicated in the 3′ UTR of an antibiotic resistance transcript and may be directly sequenced in situ (FIG. 43).

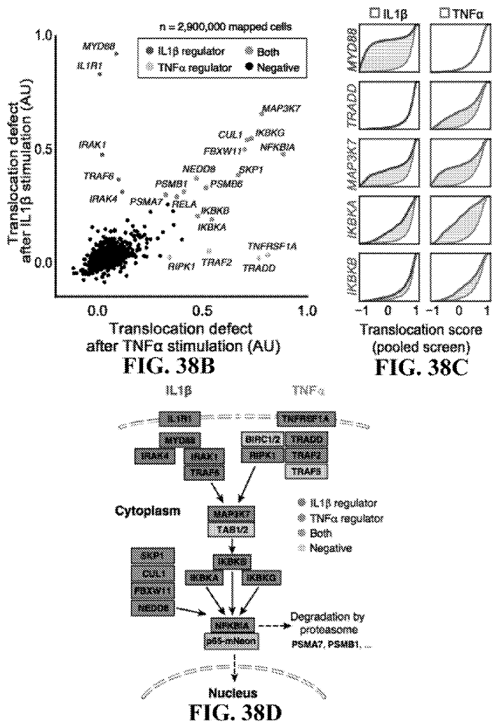

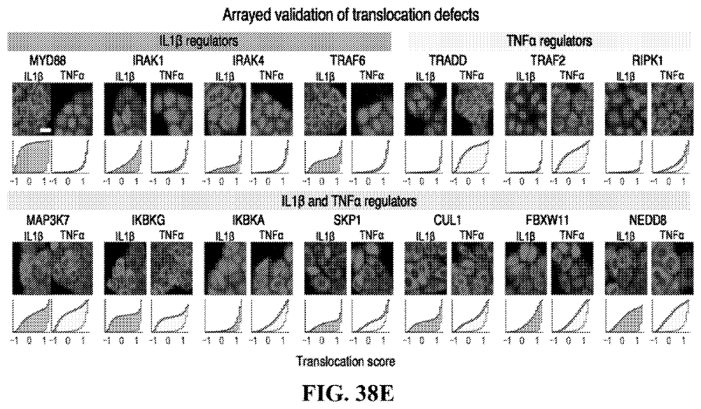

Finally, a large screen was performed to identify genes required for NF-κB activation, an extensively studied pathway that serves as a model for dynamic signaling in cells16. A system was relied on where stimulation of HeLa-TetR-Cas9-p65-mNeonGreen cells with either TNFα or IL1(3, cytokines that activate NF-κB via alternate pathways, leads to robust p65-mNeonGreen nuclear translocation (FIG. 38A). A library of 3,063 sgRNAs targeting a set of 960 genes encompassing known NF-κB pathway components as well as all GO-annotated ubiquitin ligases and deubiquitination enzymes was screened, which are intimately involved in both positive and negative regulation of NF-κB activation (FIG. 38A). After stimulation with either TNFα or IL1β, the degree of p65-mNeonGreen translocation in each cell was scored, and ranked the perturbations by their translocation score distribution compared to that of the negative control guides to identify hits specific to TNFα or IL1β as well as shared regulators (FIG. 38B, 38C). The full primary screen dataset was collected from a single multi-well plate, in which a total of 8,168,177 cells were imaged, with 3,037,909 cells retained for analysis after filtering cells based on reporter expression, nuclear morphology, and exact barcode mapping.

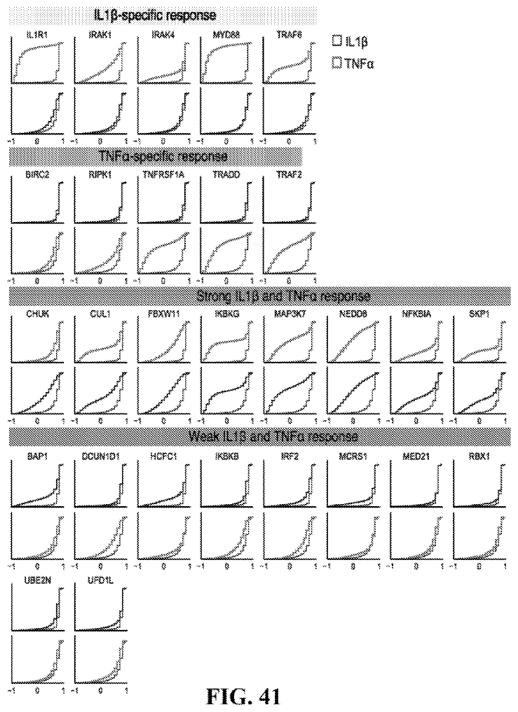

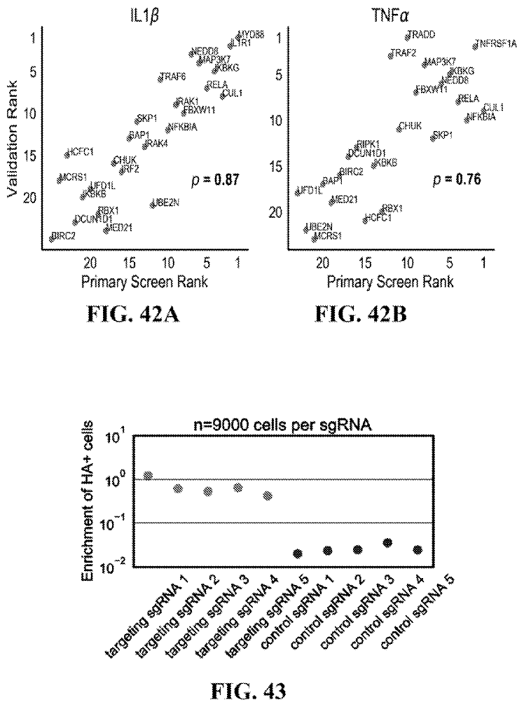

The screen recovered known pathway components annotated for NF-κB activation by IL1β (10/12 genes, KEGG) and TNFα (11/15 genes), including the cytokine-specific receptors, adapter proteins, and factors that activate the shared upstream regulator MAP3K7 (FIG. 38D)17. Hits common to both cytokines included MAP3K7 itself and its target, the IKK complex (IKBKA/B/G), as well as components of the SKP1-CUL1-F-box ubiquitin ligase complex and proteasome subunits, which together lead to degradation of the inhibitory IκBα protein and nuclear translocation of p65. Validation with arrayed CRISPR knockouts confirmed 20/20 top-ranked hits (FIG. 38E and FIG. 41), as well as potentially novel hits BAP1, HCFC1, DCUN1D1, RBX1, and MCRS1. The high sensitivity of the primary screen was underscored by the validation of genes associated with NF-κB activation that showed with weak effects on p65-mNeon translocation, including RIPK1, BIRC2 and IRF2 (FIG. 41). Phenotype strength was well correlated between the validation and the primary screening data (Spearman's ρ=0.87 (IL1β), ρ=0.76 (TNFα)), emphasizing the quantitative nature of the primary screen (FIG. 42A-42B). The failure to detect the KEGG-annotated genes TAB1 and TAB2 may be due to their non-essentiality for NF-kB signaling 18.

Pooled optical screens are a novel method for systematic analysis of the genetic components underpinning a broad palette of spatially and temporally defined phenotypes. The workflow closely mimics conventional pooled screening and requires no specialized hardware apart from a standard automated epifluorescence microscope. Optical demultiplexing is compatible with any perturbation that can be identified by a short sequence, including Cas9-mediated gene knockout, repression, and activation, as well as libraries of in silico-designed peptides, ORFs or regulatory elements barcoded with expressed tags19,20.

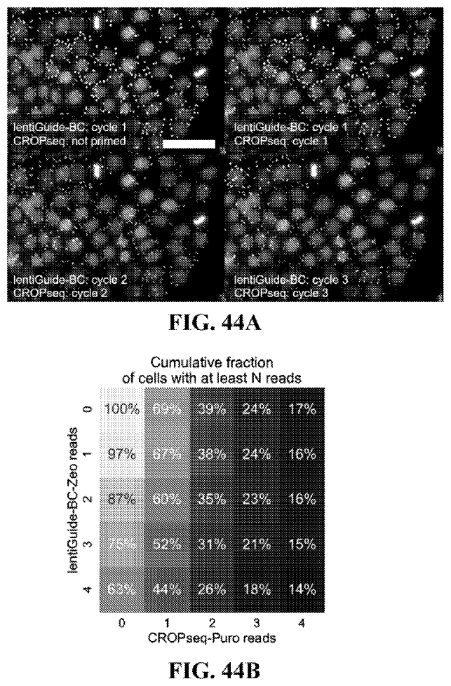

This approach has broad applicability in many settings. For example, multiple perturbation barcodes can be read out within the same cell (FIG. 44), suggesting a straightforward route to studying genetic interactions by microscopy. The potential to integrate optical screening with high-dimensional morphological profiling and in situ multiplexed gene expression analysis8,21-24 raises the prospect of learning phenotypes from data rather than pre-specifying phenotypes of interest and validating assays for their measurement. Existing protocols for in situ sequencing in tissue samples8,25 also highlights the exciting possibility of perturbing cells in vivo and measuring phenotypes within the native spatial context.

### References for Example 6

1. Wang, Tim, et al. “Genetic screens in human cells using the CRISPR-Cas9 system.” Science 343.6166 (2014): 80-84.
2. Shalem, Ophir, et al. “Genome-scale CRISPR-Cas9 knockout screening in human cells.” Science 343.6166 (2014): 84-87.
3. Parnas, Oren, et al. “A genome-wide CRISPR screen in primary immune cells to dissect regulatory networks.” Cell 162.3 (2015): 675-686.
4. Datlinger, Paul, et al. “Pooled CRISPR screening with single-cell transcriptome readout.” Nature methods 14.3 (2017): 297.
5. Jaitin, Diego Adhemar, et al. “Dissecting immune circuits by linking CRISPR-pooled screens with single-cell RNA-seq.” Cell 167.7 (2016): 1883-1896.
6. Dixit, Atray, et al. “Perturb-Seq: dissecting molecular circuits with scalable single-cell RNA profiling of pooled genetic screens.” Cell 167.7 (2016): 1853-1866.
7. Adamson, Britt, et al. “A multiplexed single-cell CRISPR screening platform enables systematic dissection of the unfolded protein response.” Cell 167.7 (2016): 1867-1882.
8. Hill, Andrew J., et al. “On the design of CRISPR-based single-cell molecular screens.” Nature methods 15.4 (2018): 271.
9. Xie, Shiqi, et al. “Frequent sgRNA-barcode recombination in single-cell perturbation assays.” PloS one 13.6 (2018): e0198635.
10. Adamson, Britt, et al. “Approaches to maximize sgRNA-barcode coupling in Perturb-seq screens.” bioRxiv (2018): 298349.
11. Feldman, David, et al. “Lentiviral co-packaging mitigates the effects of intermolecular recombination and multiple integrations in pooled genetic screens.” bioRxiv (2018): 262121.
12. Lawson, Michael J., et al. “In situ genotyping of a pooled strain library after characterizing complex phenotypes.” Molecular systems biology 13.10 (2017): 947.
13. Emanuel, George, Jeffrey R. Moffitt, and Xiaowei Zhuang. “High-throughput, image-based screening of pooled genetic-variant libraries.” nature methods 14.12 (2017): 1159.
14. Carpenter, Anne E., et al. “CellProfiler: image analysis software for identifying and quantifying cell phenotypes.” Genome biology 7.10 (2006): R100.
15. Ke, Rongqin, et al. “In situ sequencing for RNA analysis in preserved tissue and cells.” Nature methods 10.9 (2013): 857.
16. Chen, Xiaoyin, et al. “Efficient in situ barcode sequencing using padlock probe-based BaristaSeq.” Nucleic acids research (2017).
17. Lee, Je Hyuk, et al. “Highly multiplexed subcellular RNA sequencing in situ.” Science 343.6177 (2014): 1360-1363.
18. Chen, Kok Hao, et al. “Spatially resolved, highly multiplexed RNA profiling in single cells.” Science 348.6233 (2015): aaa6090.
19. Lubeck, Eric, et al. “Single-cell in situ RNA profiling by sequential hybridization.” Nature methods 11.4 (2014): 360.
20. Gewurz, Benjamin E., et al. “Genome-wide siRNA screen for mediators of NF-κB activation.” Proceedings of the National Academy of Sciences 109.7 (2012): 2467-2472.
21. Sack, Laura Magill, et al. “Sources of error in mammalian genetic screens.” G3: Genes, Genomes, Genetics 6.9 (2016): 2781-2790.
22. Yang, Xiaoping, et al. “A public genome-scale lentiviral expression library of human ORFs.” Nature methods 8.8 (2011): 659.
23. Kanehisa, Minoru, and Susumu Goto. “KEGG: kyoto encyclopedia of genes and genomes.” Nucleic acids research28.1 (2000): 27-30.
24. Shim, Jae-Hyuck, et al. “TAK1, but not TAB1 or TAB2, plays an essential role in multiple signaling pathways in vivo.” Genes & development 19.22 (2005): 2668-2681.
25. Buschmann, Tilo, and Leonid V Bystrykh. “Levenshtein Error-Correcting Barcodes for Multiplexed DNA Sequencing.” BMC Bioinformatics 14 (2013): 272. doi:10.1186/1471-2105-14-272.
26. Wang, Xiao, et al. “Three-dimensional intact-tissue sequencing of single-cell transcriptional states.” Science (2018): eaat5691.
27. Melnikov, Alexandre, et al. “Systematic dissection and optimization of inducible enhancers in human cells using a massively parallel reporter assay.” Nature biotechnology 30.3 (2012): 271.

### Methods

### Tissue Culture

HEK293FT cells (ATCC CRL-1573) were cultured in DMEM with sodium pyruvate and GlutaMAX (Life 10569044) supplemented with heat-inactivated fetal bovine serum (Seradigm 97068-085) and 100 U/mL penicillin-streptomycin (Life Technologies 15140163). All HeLa cell lines were cultured in the same media with serum substituted for 10% tetracycline-screened fetal bovine serum (Hyclone SH30070.03T).

Parental HeLa-TetR-Cas9 cells were a gift. In order to select an optimal clone for further experiments, single cells were sorted into a 96-well plate (Sony FACSAria), clonally expanded, and screened for Cas9 activity after 8 days with and without 1 μg/mL doxycycline induction. Cas9 activity was assessed by transducing each clone with pXPR_011 (Addgene #59702), a GFP reporter plasmid, and using FACS to read out efficiency of genome editing. Additionally, genomic DNA was extracted from both uninduced and induced clones by resuspending in cell lysis buffer (10 mM Tris pH 7.5, 1 mM CaCl2, 2 mM MgCl2, 1 mM EDTA, and 0.2 mg/mL Proteinase K), and heating for 10 minutes at 65° C. and 15 minutes at 95° C. The guide target region was amplified by PCR and sequenced on an Illumina Miniseq. The best clones showed efficient indel generation (≥97%) in the presence of doxycycline and minimal cutting (≤2%) in its absence.

In preparation for in situ analysis, cells were seeded onto glass-bottom plates (6-well: Cellvis P06-1.5H-N, 24-well: Greiner Bio-one 662892, 96-well: Greiner Bio-one 665892) at a density of 50,000/cm2 and incubated for 2 days to permit proper cell attachment, spreading, and colony formation.

### Lentiviral Production

HEK293FT cells were seeded into 15-cm plates or multi-well plates at a density of 50,000/cm2. After 16-20 h, cells were transfected with pMDG, psPAX, and a lentiviral transfer plasmid (2:3:4 mass ratios) using Lipofectamine 3000. Media was exchanged after 4 hours and supplemented with 2 mM caffeine 20 h post-transfection. Viral supernatant was harvested 48 h after transfection and filtered through 0.45 μm PVDF filters (Millipore SLHVR04NL).

### Lentiviral Transduction

HeLa-TetR-Cas9 cells were transduced by adding viral supernatant supplemented with polybrene (8 μg/mL) and centrifuging at 1000 g for 2 hours at 33° C. At 5 h post-infection, media was exchanged. At 24 h post-infection, cells were passaged into media containing selection antibiotic (1 μg/mL puromycin; 300 μg/mL hygromycin; 300 μg/mL zeocin).

For lentiviral transduction of barcoded perturbation libraries, a carrier plasmid was utilized to minimize recombination between distant genetic elements (e.g., sgRNA and associated barcode). Libraries were packaged following the above protocol, with the library transfer plasmid diluted in integration-deficient carrier vector pR_LG (1:10 mass ratio of library to carrier).

### Library Cell Line Validation

For library transductions, multiplicity of infection was estimated by counting colonies after sparse plating and antibiotic selection. Genomic DNA was also extracted for NGS validation of library representation.

### Next Generation Sequencing of Libraries

Genomic DNA was extracted using an extraction mix as described above. Barcodes and sgRNAs were amplified by PCR from a minimum of 1e6 genomic equivalents per library using NEBNext 2× Master Mix (initial denaturation 5 min at 98° C., followed by 28 cycles of annealing for 10 s at 65° C., extension for 25 s at 72° C., and denaturation for 20 s at 98° C.).

### Library Design and Cloning

A set of 12-nt barcodes was designed by selecting 80,000 barcodes from the set of 16.7 million possible 12-nt sequences by filtering for GC content between 25% and 75%, no more than 4 consecutive repeated bases, and minimum substitution and insertion/deletion edit distance (Levenshtein distance) of 3 between any pair of barcodes. Ensuring a minimum edit distance is useful for detecting and correcting errors, which arise mainly from DNA synthesis and in situ reads with low signal-to-background ratios. The E-CRISP web tool was used to select sgRNA sequences targeting genes of interest. Barcode-sgRNA pairs were randomly assigned and co-synthesized on a 130-nt 90K oligo array (CustomArray). Individual libraries were amplified from the oligo pool by dial-out PCR (Schwartz 2012) and cloned into lentiGuide-BC-CMV via two steps of Golden Gate assembly using BsmBI and BbsI restriction sites. Libraries were transformed in electrocompetent cells (Lucigen Endura) and grown in liquid culture for 18 h at 30° C. before extracting plasmid DNA. The sgRNA-barcode association was validated by Sanger sequencing individual colonies from the final library.

### Padlock-Based RNA Detection

Preparation of targeted RNA amplicons for in situ sequencing was adapted from published protocols with modifications to improve molecular detection efficiency and amplification yield [Ke et al 2013, Larsson 2010]. Cells were fixed with 4% paraformaldehyde (Electron Microscopy Sciences 15714) for 30 minutes, washed with PBS, and permeabilized with 70% ethanol for 30 minutes. Permeabilization solution was carefully exchanged with PBS-T wash buffer (PBS+0.05% Tween-20) to minimize sample dehydration. Reverse transcription mix (1× RevertAid RT buffer, 250 μM dNTPs, 0.2 mg/mL BSA, 1 μM RT primer, 0.8 U/μL Ribolock RNAse inhibitor, and 4.8 U/μL RevertAid H minus reverse transcriptase) was added to the sample and incubated for 16 h at 37° C. Following reverse transcription, cells were washed 5 times with PBS-T and postfixed with 3% paraformaldehyde and 0.1% glutaraldehyde for 30 minutes at room temperature, then washed with PBS-T 5 times. At this stage, cells expressing p65-mNeonGreen were imaged. Samples were thoroughly washed again with PBS-T, incubated in a padlock probe and extension-ligation reaction mix (1× Ampligase buffer, 0.4 U/μL RNAse H, 0.2 mg/mL BSA, 10 nM padlock probe, 0.02 U/μL Stoffel fragment, 0.5 U/μL Ampligase and 5 nM dNTPs) for 5 minutes at 37° C. and 90 minutes at 45° C., and then washed 2 times with PBS-T. Circularized padlocks were amplified with rolling circle amplification mix (1× Phi29 buffer, 250 μM dNTPs, 0.2 mg/mL BSA, 5% glycerol, and 1 U/μL Phi29 DNA polymerase) at 30° C. for either 3 h or overnight.

### In Situ Sequencing

Rolling circle amplicons were prepared for sequencing by hybridizing a mix containing sequencing primer pL42_FWD or CROP_FWD (1 μM primer in 2×SSC+10% formamide) for 30 min at room temperature. Barcodes were read out using sequencing-by-synthesis reagents from the Illumina Miseq 500 cycle Nano Kit (Illumina MS-103-1003). First, samples were washed with incorporation buffer (Nano kit PR2) and incubated for 3 minutes in incorporation mix (Nano kit reagent 1) at 60° C. Samples were then thoroughly washed with PR2 at 60° C. (6 washes for 3 minutes each) and placed in 200 ng/mL DAPI in 2×SSC for fluorescence imaging. Following each imaging cycle, dye terminators were removed by incubation for 6 minutes in Illumina cleavage mix (Nano kit reagent 4) at 60° C. and samples were thoroughly washed with PR2.

### Fluorescence Microscopy

All screening images were acquired using a Ti-E Eclipse inverted epifluorescence microscope (Nikon) with automated XYZ stage control and hardware autofocus. An LED light engine (Lumencor Sola SE FISH II) was used for fluorescence illumination and all hardware was controlled using Micromanager software (Edelstein 2010). In situ sequencing cycles with the Illumina Miseq kit were imaged using a 10×0.45 NA CFI Plan Apo Lambda objective (Nikon) with the following filters (Semrock) and exposure times for each base: G (excitation 534/20 nm, emission 572/28 nm, dichroic 552 nm, 200 ms); T (excitation 589/15 nm, emission 628/32 nm, dichroic 593 nm, 200 ms exposure); A (excitation 640/14 nm, emission 676/29 nm, dichroic 635 nm, 200 ms); C (excitation 661/20 nm, emission 725/40 nm, dichroic 695 nm, 800 ms).

### Frameshift Reporter Screen/Frameshift Reporter Cells

HeLa-TetR-Cas9 cells were stably transduced at MOI>2 with pL_FR_Hygro and selected with hygromycin for 7 days to generate the HeLa-TetR-Cas9-FR cell line. Cells transduced with the pL_FR_Hygro lentiviral vector express an open reading frame consisting of a 50-nt frameshift reporter target sequence, followed by an H2B histone gene with C-terminus HA epitope tag (+1 frameshift), followed by a second H2B gene with C-terminus myc tag (+0 frameshift) and hygro antibiotic resistance gene (+0 frameshift). All genes are preceded by self-cleaving 2A peptides in the same reading frame. Before generation of Cas9-mediated indel mutations, cells translate the genes with +0 frameshift. Subsequent activation of the reporter by co-expression of Cas9 and a targeting sgRNA leads to mutations in the target sequence, which may alter the downstream reading frame. A frameshift of +1 leads to expression of the H2B-HA protein, which can be visualized by immunofluorescence and detected by microscopy or flow cytometry. Integration of multiple copies of reporter per cell increases the likelihood of generating a +1 frameshift in at least one copy.

HeLa-TetR-Cas9-FR cells were used to screen targeting and control sgRNAs. A barcoded sgRNA perturbation library (pLL31) with 1,000 barcodes, each encoding one of 5 targeting or 5 control sgRNAs, was synthesized and cloned into lentiGuide-BC. This library was transduced into HeLa-TetR-Cas9-FR cells at MOI<0.05 in three replicates, which were independently cultured and screened. Following 4 days of puromycin selection, cells were collected to validate library representation by NGS. Cas9 expression was induced by supplementing the culture media with 1 μg/mL doxycycline for 6 days. Cells were then split for screening either via in situ sequencing or by FACS.

For in situ screening, 500,000 cells were seeded into each well of a glass-bottom 6-well plate (CellVis). After two days of culture, in situ padlock detection and sequencing were carried out as above, with the modification that prior to sequencing-by-synthesis, cells were immunostained to monitor reporter frame shifts by blocking and permeabilizing with 3% BSA+0.5% Triton X-100 for 5 minutes, incubating in rabbit anti-HA (1:1000 dilution in 3% BSA) for 30 minutes, washing with PBS-T and incubating with goat anti-rabbit F(ab′)2 fragment Alexa 488 (CST 4412S, 1:1000 dilution in 3% BSA) for 30 minutes. Samples were changed into imaging buffer (200 ng/mL DAPI in 2×SSC) and phenotype images were acquired.

FACS screening was carried out by fixing cells with 4% PFA, permeabilizing with 70% ice-cold ethanol, and immunostaining with the same anti-HA primary and secondary antibodies and dilutions used for in situ analysis. Cells were sorted into HA+ and HA-populations (Sony SH800) and genomically integrated perturbations were sequenced as described above. An enrichment score for each barcoded perturbation was defined as the log ratio of normalized read counts.

### NF-κB Pooled Screen

HeLa-TetR-Cas9 cells were transduced with pR14p65-mNeonGreen, a C-terminal fusion of p65 with a bright monomeric green fluorescent protein (Allele Biotechnology). Fluorescent cells were sorted by FACS (Sony FACSAria) and re-sorted to select for cells with stable expression. This reporter cell line was further transduced with either a 1,000-barcode sgRNA library (pLL31, 87 genes targeted) or a 4,000-barcode sgRNA library (pLL47, 980 genes targeted). Cells were selected with puromycin for 4 days and library representation was validated by NGS.

Cas9 expression was induced with 1 μg/mL doxycycline and cells were seeded onto 6-well glass-bottom plates 2 days prior to translocation experiments. The total time between Cas9 induction and performing the NF-κB activation assay was 7 days. Cells were stimulated with either 30 ng/mL TNFα or 30 ng/mL IL1β for 40 minutes prior to fixation with 4% paraformaldehyde for 30 minutes and initiation of the in situ sequencing protocol. Translocation phenotypes were recorded after the post-fixation step by exchanging for imaging buffer (2×SSC+200 ng/mL DAPI) and imaging the nuclear DAPI stain and p65-mNeonGreen. After phenotyping, the remainder of the in situ sequencing protocol (gap-fill and RCA) and 12 bases of sequencing-by-synthesis were completed.

### Image Analysis

Images of cell phenotype and in situ sequencing of perturbations were aligned using cross-correlation of DAPI-stained nuclei. Nuclei were detected using local thresholding and watershed-based segmentation. Cells were typically segmented using local thresholding of cytoplasmic stain and assignment of pixels to the nearest nucleus by the fast-marching method. Frameshift reporter and NF-κB translocation phenotypes were quantified by calculating pixel-wise correlations between the nuclear DAPI channel and 488 channel (HA stain or p65-mNeonGreen, respectively).

Sequencing reads were detected by applying a Laplacian-of-Gaussian linear filter, calculating the per-pixel standard deviation over sequencing cycles, averaging over color channels, and finding local maxima. The base intensity at each cycle was defined as the maximum value in a 3×3 pixel window centered on the read. A linear transformation was then applied to correct for optical cross-talk and variable intensity between color channels. Finally, each base was called according to the channel with maximum corrected intensity, and a per-base quality score was defined as the ratio of intensity for the maximum channel to total intensity for all channels. The output of the sequencing imaging analysis was a FASTQ file recording each sequencing read along with the identity of the overlapping cell, quality score per base and spatial location. Python scripts, including a complete pipeline from example data to sequencing reads, will be made available on Github.

TABLE 8
Oligo sequences used for padlock detection and sequencing-by-synthesis.
Sequences of reverse transcription primer (pRT), padlock probe (pPD),
and sequencing-by-synthesis primer (pSBS) used for detecting barcodes
in cells transduced with the lentiGuide-BC or CROPseq vectors.
Note that LNA-modified bases in the reverse transcription primer are
essential to retain the linkage between the cDNA and fixed RNA,
preventing diffusion of cDNA out of the cell.

| name | Sequence | modifications | 1717 NO |
|---|---|---|---|
| pRT_lentiGuide-BC | G+AC+GT+GT+GC+TT+AC+CCAAA | LNA bases | 17 |
| | GG | | |
| pRT_CROPseq | G+AC+TA+GC+CT+TA+TT+TTAAC | LNA bases | 18 |
| | TTGCTAT | | |
| pPD_lentiGuide-BC | /5Phos/actggctattcattcgcCTCCTGTTCG | 5′phosphorylation | 19 |
| | ACAGTCAGCCGCATCTGCGTCTATT | | |
| | TAGTGGAGCCC | | |
| | TTGtgttcaatcaacattcc | | |
| pPD_CROPseq | /5Phos/gttttagagctagaaatagcCTCCTGTT | 5′phosphorylation | 20 |
| | CGACAGTCAGCCGCATCTGCGTCT | | |
| | ATTTAGTGGAG | | |
| | CCCTTGaaggacgaaacaccg | | |
| pSBS_lentiGuide-BC | TTCGACAGTCAGCCGCATCTGCGT | | 21 |
| | CTATTTAGTGG | | |
| | AGCCCTTGtgttcaatcaacattcc | | |
| pSBS_CROPseq | TCAGCCGCATCTGCGTCTATTTAG | | 22 |
| | TGGAGCCCTTG | | |
| | Aaggacgaaacaccg | | |

TABLE 9
Screening throughput. The screening approach described in this study uses
fully pooled protocols for library construction, cloning and cell culture. As a result, practical
limitations to screen scale come mainly from the time required to image large numbers of cells,
and the cost of reagents for padlock detection and in situ sequencing. Table 9 summarizes the
scale and throughput of the NF-kB screen performed, as well as two hypothetical screens using
either low-density cell culture or a genome-scale perturbation library. The key figure
determining both throughput and cost is the total surface area processed, which affects the
volume of reagents used and the time per sequencing cycle. The NF-kB screen was read out
from a total of ~8 million cells in a single 6-well plate.

| | perturbations | screen | cell | mapped | cells | cell density | sequencing | #of | total readout | reagent cost |
|---|---|---|---|---|---|---|---|---|---|---|
| | (e.g., sgRNAs) | replicates | coverage | cells | (total) | (cells/cm ^{∧}2) | cycle (hours) | cycles | time (days) | (direct) |
| NF-kB screen | 4,000 | 6 | 130 | 3,120,000 | 8,320,000 | 175,000 | 6 | 12 | 3 | $1,485 |
| in this study | | | | | | | | | | |
| (3 replicates each | | | | | | | | | | |
| for IL1b and TNFa) | | | | | | | | | | |
| 4000 perturbation | 4,000 | 3 | 150 | 1,800,000 | 4,800,000 | 25,000 | 21 | 12 | 11 | $5,997 |
| screen with low- | | | | | | | | | | |
| density cells | | | | | | | | | | |
| Genome-scale | 60,000 | 3 | 200 | 36,000,000 | 96,000,000 | 175,000 | 59 | 12 | 30 | $17,134 |
| CRISPR screen | | | | | | | | | | |
| with 3 replicates | | | | | | | | | | |

TABLE 10
Reagent cost estimate for in situ barcode readout. The major reagent
costs (direct) are shown. The cost per 6-well plate corresponds
to the NF-kB screen in FIG. 38 and the first row of Table 9.

| | | cost/6-well |
|---|---|---|
| | | plate (Table |
| step | reagent | S1 row 1) |
| reverse transcription | RevertAID H minus RT | $192.42 |
| reverse transcription | RNaseIn | $55.62 |
| padlock extension/ligation | RnaseH | $81.60 |
| padlock extension/ligation | TaqIT | $0.60 |
| padlock extension/ligation | Ampligase | $97.98 |
| rolling circle amplification | Φ29 polymerase | $441.00 |
| | padlock detection | $869.22 |
| sequencing by synthesis | Illumina MiSeq v2: | $52.50 |
| | incorporation mix, | |
| | cleavage mix, wash buffer | |
| | total (12 sequencing cycles) | $1,4199.22 |

TABLE 11
Top NF-κB screen hits. Each gene is assigned a score for lack
of p65-mNeon translocation in response to IL1β and TNFα
stimulation. The top thirty genes are shown, ordered by the
minimum of the rank for each cytokine score.

| | | | IL1β | TNFα |
|---|---|---|---|---|
| | IL1β | TNFα | p-value | p-value |
| gene | score | score | (corrected) | (corrected) |
| MYD88 | 0.92 | 0.09 | | |
| NFKBIA | 0.48 | 0.89 | | |
| IL1R1 | 0.83 | 0.01 | | |
| TNFRSF1A | 0.04 | 0.81 | | |
| MAP3K7 | 0.66 | 0.78 | | |
| IKBKG | 0.55 | 0.73 | | |
| TRADD | 0.02 | 0.77 | | |
| CUL1 | 0.54 | 0.72 | | |
| FBW11 | 0.50 | 0.70 | | |
| IRAK1 | 0.48 | 0.02 | | |
| SKP1 | 0.39 | 0.67 | | |
| CHUK | 0.19 | 0.54 | | |
| TRAF2 | 0.06 | 0.53 | | |
| NEDD8 | 0.38 | 0.47 | | |
| PSMB6 | 0.33 | 0.52 | | |
| TRAF6 | 0.37 | 0.10 | | |
| IKBKB | 0.21 | 0.48 | | |
| PSMB1 | 0.32 | 0.41 | | |
| IRAK4 | 0.32 | 0.12 | | |
| PSMC2 | 0.11 | 0.40 | | |
| PSMA7 | 0.30 | 0.32 | | |
| RELA | 0.30 | 0.38 | | |
| UBA52 | 0.17 | 0.36 | | |
| RBM25 | 0.26 | 0.33 | | |
| RBX1 | 0.23 | 0.36 | | |
| RIPK1 | 0.03 | 0.34 | | |
| COPS5 | 0.23 | 0.25 | | |
| GLTSCR2 | 0.14 | 0.31 | | |
| PSMA4 | 0.19 | 0.14 | | |
| ACTL6A | 0.03 | 0.30 | | |

### Additional References for Example 6

1. Chen, Xiaoyin, et al. “Efficient in situ barcode sequencing using padlock probe-based BaristaSeq.” Nucleic acids research (2017).
2. Lee, Je Hyuk, et al. “Highly multiplexed subcellular RNA sequencing in situ.” Science 343.6177 (2014): 1360-1363.
3. Edelstein, Arthur, et al. “Computer control of microscopes using ¬μManager.” Current protocols in molecular biology (2010): 14-20.
4. Schwartz, Jerrod J., Choli Lee, and Jay Shendure. “Accurate gene synthesis with tag-directed retrieval of sequence-verified DNA molecules.” Nature methods 9.9 (2012): 913.
5. Larsson, Chatarina, et al. “In situ detection and genotyping of individual mRNA molecules.” Nature methods 7.5 (2010): 395.

### Example 7—Highly Scalable Mapping of Phenotype to Genotype in an Optical Screen

The screening approach described in this study uses fully pooled protocols for library construction, cloning, and cell culture. As a result, practical limitations to screen scale come mainly from the time required to image large numbers of cells and the cost of reagents for padlock detection and in situ sequencing.

Applicants developed an optical barcoding strategy to enable pooled screens of microscopy-based phenotypes (FIG. 39A). In the approach, perturbations are identified via targeted in situ sequencing of an expressed barcode, read out by fluorescence microscopy (FIG. 35B). As pooled screens in mammalian cells generally use single-copy lentiviral integration to deliver perturbations, Applicants focused on establishing reliable in situ sequencing of barcode transcripts in this format. To apply this barcoding method to CRISPR-based screens, Applicants modified an existing lentiviral CRISPR guide RNA (sgRNA) expression vector (lentiGuide-Puro) to express both an sgRNA and a 12-nt barcode, and termed the resulting vector lentiGuide-BC. The barcodes and constant flanking sequences were inserted into the 3′ UTR of the Pol II-transcribed antibiotic resistance gene, a highly expressed mRNA suitable for in situ detection.

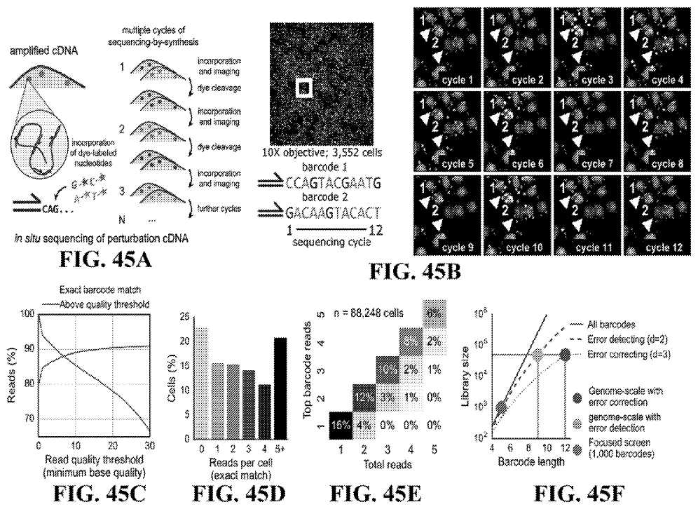

To test in situ identification of perturbations, and as discussed in Example 6, Applicants transduced a lentiGuide-BC library containing 40 sgRNA-barcode pairs into HeLa-TetR-Cas9 cells at low multiplicity of infection (MOI). Applicants prepared samples for targeted in situ sequencing using a padlock-based approach (Ke et al., 2013), in which variable sequences within an RNA transcript are converted to single-stranded DNA and enzymatically amplified via in situ reverse transcription, padlock extension/ligation, and rolling circle amplification (RCA) (FIGS. 35B, 45A, FIG. 39). Sequencing the amplified DNA with a 4-color sequencing-by-synthesis chemistry over 12 cycles generated high quality reads with excellent uniformity among barcodes (FIGS. 45B and 45C). After image segmentation and base calling analysis, >85% of sequence reads mapped exactly to one of the 40 known barcodes out of the 412=16.7 million possible 12-nt sequences (FIG. 45D, STAR Methods).

Perturbation Detection In Situ is Compatible with the Demands of Large Screens

Next, Applicants demonstrated that an in situ sequencing approach can meet the demands of large pooled screens: (1) measurement across millions of cells quickly; (2) high specificity and sensitivity per cell; (3) detection of one or more perturbations delivered independently; and (4) compatibility with a large number of perturbations.

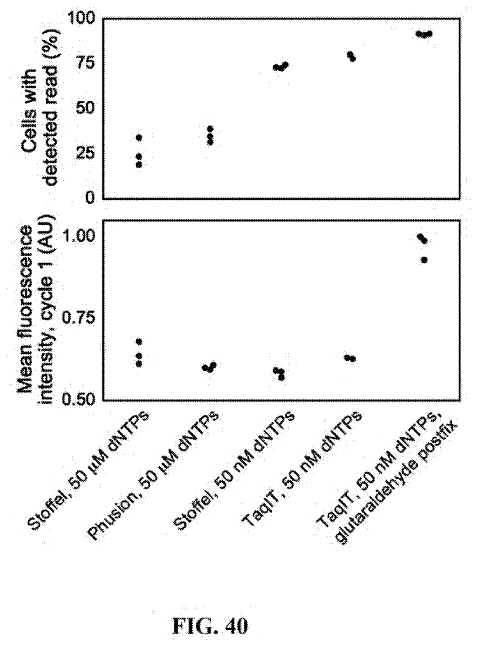

Applicants showed that the in situ readout step can process millions of cells within a few days at high coverage (typically 100-1,000 cells/perturbation), as necessary to enable high-throughput optical screening. First, high signal intensity allows accurate sequence data to be obtained across large fields of view at low optical magnification, each containing thousands of cells (FIG. 45B, Tables 10 and 12). Next, Applicants maximized fluorescence signal-to-background by optimizing the barcode amplification protocol, including the post-fixation step that follows reverse transcription and the conditions for padlock extension/ligation (FIG. 40). Using the optimized protocol, in situ sequencing spots were readily visible at 10× optical magnification, with at least one exactly mapped read detected in more than 82% of transduced cells (FIGS. 45B and 45E).

Next, Applicants constructed two types of large barcoded perturbation libraries using oligo microarray synthesis. In the first approach, Applicants designed a set of 83,314 barcodes (out of the 16.7 million possible 12-nt sequences) using criteria that balanced GC content, minimized homopolymer repeats, and maintained a minimum pairwise edit distance of three. (STAR Methods). This allows rejection of reads containing up to two errors (single base insertion/deletion/substitution) arising from oligo synthesis or in situ processing (FIG. 45F, STAR Methods) (Buschmann and Bystrykh, 2013). Applicants then used a two-step procedure to clone a library of sgRNAs and associated barcodes into lentiGuide-BC (STAR Methods). In contrast to perturbation barcoding by random pairing (Emanuel et al., 2017), this approach pairs sgRNA with specific barcodes in silico, ensuring efficient use of available barcodes. Applicants addressed swapping of barcodes and associated sgRNAs due to reverse transcription-mediated recombination during lentiviral recombination (Adamson et al., 2018; Hill et al., 2018; Sack et al., 2016; Xie et al., 2018) with a modified lentiviral packaging protocol (Feldman et al., 2018) which reduced the frequency of cells exhibiting swapped barcodes from >28% to <5%.

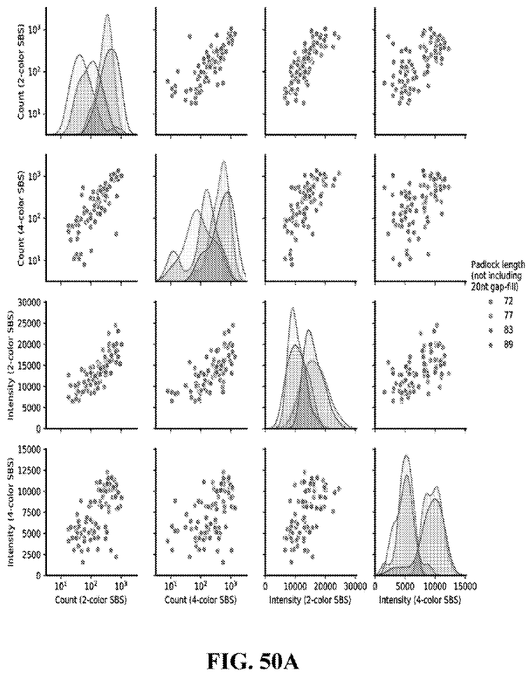

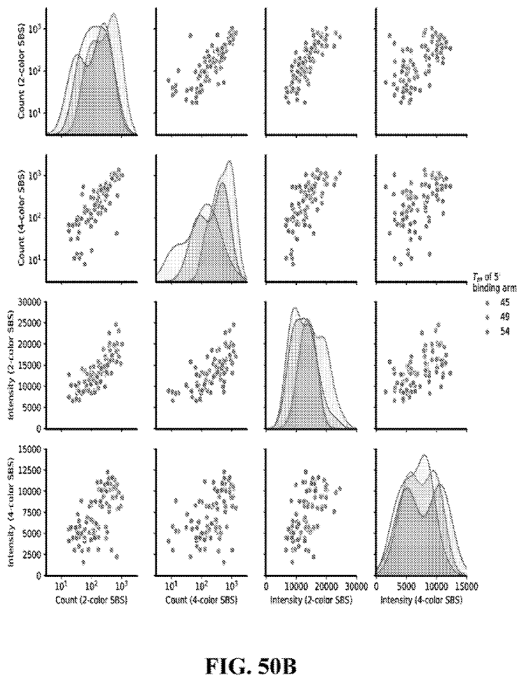

In the second approach, Applicants used the CROP-seq vector (Datlinger et al., 2017) to directly sequence a Pol II-transcribed copy of the sgRNA, rather than relying on auxiliary barcodes. Applicants observed accurate sequencing of sgRNAs in HeLa-TetR-Cas9 cells, but with reduced fluorescent signal intensity compared to lentiGuide-BC. Applicants then systematically tested a set of 84 CROP-seq-targeting padlocks with a range of padlock binding arms, backbone length, and backbone sequences (STAR Methods, FIG. 50), to yield a top-performing CROP-seq padlock with 4-fold increase in reads per cell and 1.7-fold increased signal intensity relative to the median padlock.

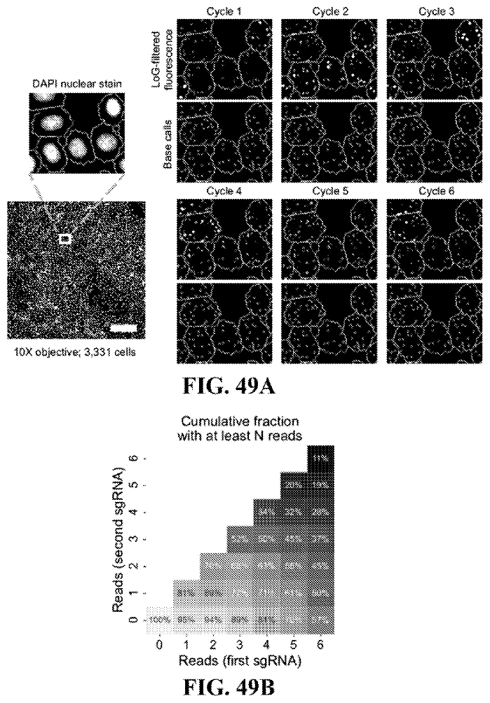

Moreover, Applicants showed that in situ sequencing could be used to read out multiple perturbations within the same cell. To this end, Applicants transduced HeLa-TetR-Cas9 cells with one CROP-seq library carrying a puromycin selection marker, followed by a second CROP-seq library carrying a zeocin selection marker. After serial transduction and antibiotic selection, Applicants performed in situ sequencing on both libraries simultaneously. Most cells (81%) contained reads mapping to two sgRNAs (FIG. 49), demonstrating that multiple perturbations can be read in one cell even when delivered by separate vectors, suggesting straightforward application to higher-order combinatorial screens.

### Accurate, Highly Scalable Mapping of Phenotype to Genotype in an Optical Pooled Screen

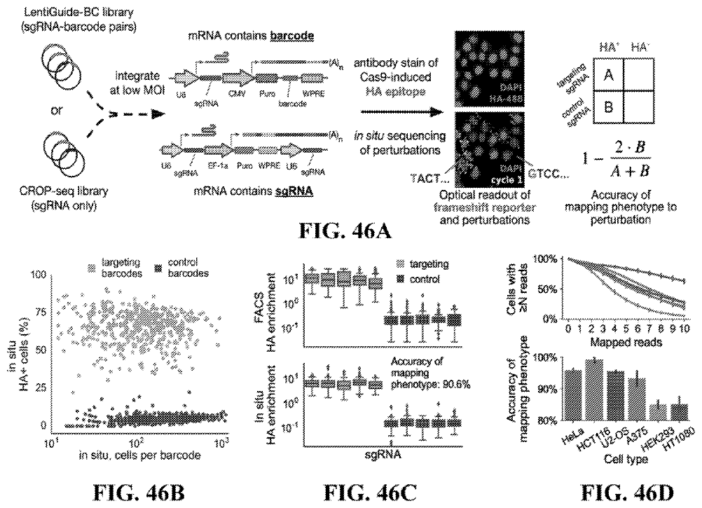

Applicants next showed that the disclosed approach can correctly map genetic perturbations to cell phenotypes in situ by performing a reporter imaging screen, in which a lentiviral reporter produces an HA-tagged, nuclear-localized H2B protein after a Cas9-induced +1 frameshift in a target region (FIG. 46A, FIG. 51, STAR Methods). Cells expressing the reporter can either be screened in situ or by FACS. The reporter is highly specific and sensitive, with a mean in situ activation across 5 targeting sgRNAs of 65±2.7% and background of <0.001% in the absence of a targeting sgRNA (FIG. 46B). Cells were transduced, stably expressing the frameshift reporter with a lentiGuide-BC library containing 972 barcodes redundantly encoding 5 targeting and 5 control sgRNAs (average of 97 barcodes per sgRNA). Applicants then induced Cas9 expression, measured reporter activation by immunofluorescence, and determined barcode sequences by in situ sequencing.

All barcodes encoding targeting sgRNAs were distinguishable from control sgRNAs by HA+ fraction, with a per-cell identification accuracy of 90.6% (based on false positive events in which HA+ cells were assigned control sgRNAs, STAR Methods). Indeed, there were no errors in barcode phenotype assignment even for barcodes represented by very few cells (FIG. 46B, STAR Methods). Screening the same cell library by FACS with NGS readout showed similar enrichment of targeting sgRNAs (FIG. 46C), with consistent representation of most barcodes in both contexts (95% within 5-fold abundance, FIG. 52). Applicants achieved comparably robust mapping of CRISPR sgRNAs to the frameshift reporter phenotype with the CROP-seq vector in HeLa cells as well as U2-OS, HCT-116, A375, HT1080 and HEK293 cells (FIG. 46D), with per-cell identification accuracy ranging from 83-98%. Thus, the combined errors that may arise from oligo synthesis, library cloning, lentiviral delivery, barcode diffusion during in situ processing, barcode readout by in situ sequencing, or incorrect assignment of reads to cells during image processing, are low enough to permit pooled functional screens.

### A Pooled Optical Screen in Millions of Cells for Regulators of NF-κB Activation

After demonstrating the ability to screen genetic knockouts for optical phenotypes, Applicants set out to identify genes required for activation of NF-κB, a family of transcription factors (p50, p52, p65, RelB and c-Rel) that translocate to the nucleus in response to a host of stimuli (Gewurz et al., 2012; Pahl, 1999). At baseline, NF-κB dimers are maintained in an inactive state in the cytoplasm by inhibitory IκB proteins, which mask nuclear localization signals to prevent NF-κB translocation into the nucleus. Upon stimulation of cell surface receptors, a signal cascade is initiated and leads to downstream activation of the IKK complex. IKK-β then phosphorylates IκB proteins, triggering their phosphorylation-dependent ubiquitination and subsequent degradation by the proteasome, releasing NF-κB from inhibition. Free NF-κB dimers may then translocate to the nucleus and induce the expression of genes promoting cell proliferation, survival and pro-inflammatory responses. It is known that post-translational modifications play a key role in regulating the baseline state and activation of NF-κB. In addition to its role in proteolytic degradation, ubiquitin has been shown to function in IKK activation and the activity of many core NF-κB pathway members is controlled by alterations in ubiquitination state.

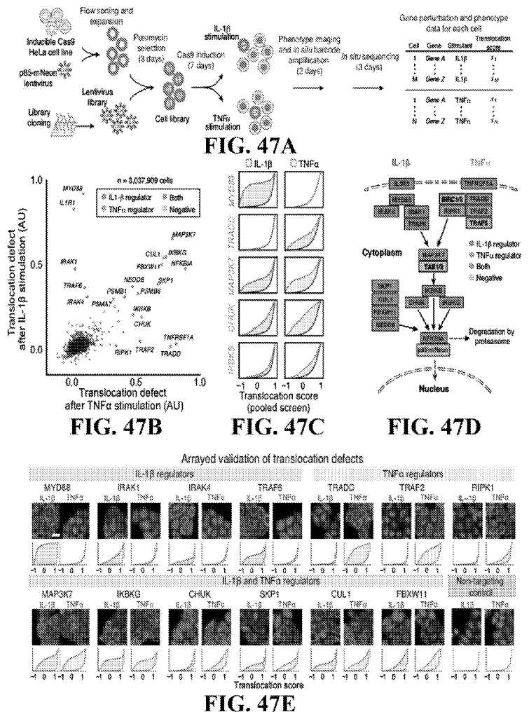

An established nuclear translocation assay was used to measure the localization of a p65-mNeonGreen reporter in HeLa cells following stimulation with either IL-1β or TNFα, cytokines that activate NF-κB via different pathways (FIG. 47A). Applicants screened 3063 sgRNAs targeting 963 genes, encompassing all GO-annotated ubiquitin ligase and deubiquitinase enzymes, and 425 immune-related genes, hypothesizing that there may be unknown roles of ubiquitin signaling in NF-κB activation and relaxation. (Chen, 2005) (FIG. 47A, Tables S4 and S5). After stimulation with either IL-1β or TNFα, Applicants imaged p65-mNeonGreen translocation, and then performed in situ sequencing of the guide barcodes. Applicants retained a total of 3,037,909 cells for analysis after filtering cells based on reporter expression, nuclear morphology, and exact barcode mapping. Applicants scored the degree of p65-mNeonGreen translocation in each cell and ranked the perturbations by the deviation of their translocation score distribution from negative control sgRNAs to identify gene knockouts that led to defects in response to IL-1β and/or TNFα (FIGS. 47B and 47C, STAR Methods).

### The Screen Recovered Most Known Regulators and Uncovered Novel Candidates

The top hits in the screen included known pathway components annotated by KEGG (Kanehisa and Goto, 2000) for NF-κB activation by IL-1β signaling (5/5 genes), TNFα signaling (4/7 genes) and downstream components (5/7 genes), including cytokine-specific receptors, adapter proteins, and factors that activate the shared regulator MAP3K7 (FIG. 47D) (Gewurz et al., 2012). Hits common to both cytokines included MAP3K7 and its target, the IKK complex (CHUK, IKBKB, IKBKG), as well as components of the SKP1-CUL1-F-box ubiquitin ligase complex and proteasome subunits, which together promote degradation of the inhibitor NFKBIA/IκBα and nuclear translocation of p65. Many of these annotated regulators were also top hits when the screen was repeated with an antibody against endogenous p65 in HeLa (16/23 KEGG genes detected), A549 (18/23 genes) and HCT116 cells (11/23 genes).

The p65-mNeon screen showed high sensitivity, successfully detecting genes involved in NF-κB activation with modest but reproducible translocation defects in the model system, such as RBCK1/HOIL1 (LUBAC complex, poly-ubiquitination of IKBKG/NEMO and RIPK1), UBE2N (activation of MAP3K7), DCUN1D1 (neddylation of CUL1), COPS5 (COPS signalosome, regulates neddylation), PADI2 (citrullination promoting nuclear import of p65) (Sun et al., 2017), UFD1L/p97 and VPRBP/DCAF1 (ubiquitin-dependent degradation of NFKBIA/IκBα) (Li et al., 2014).

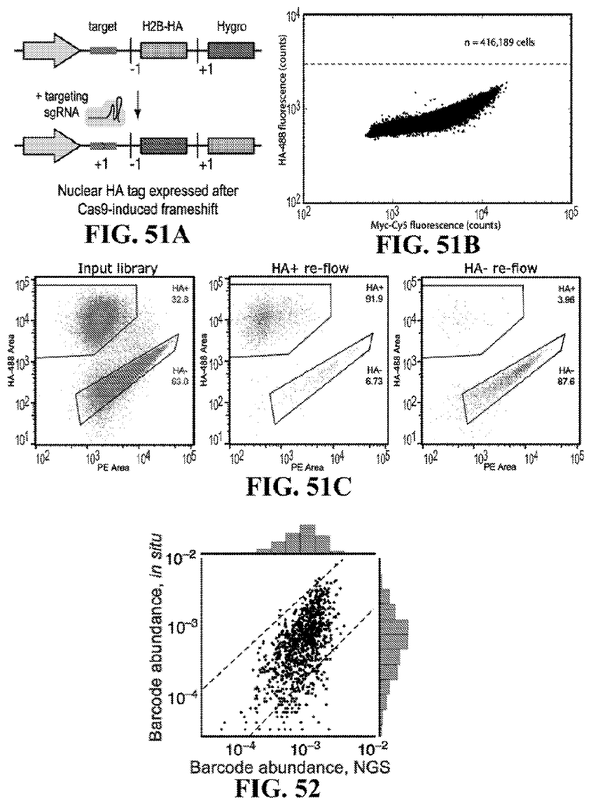

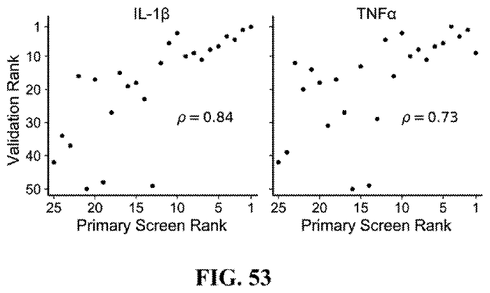

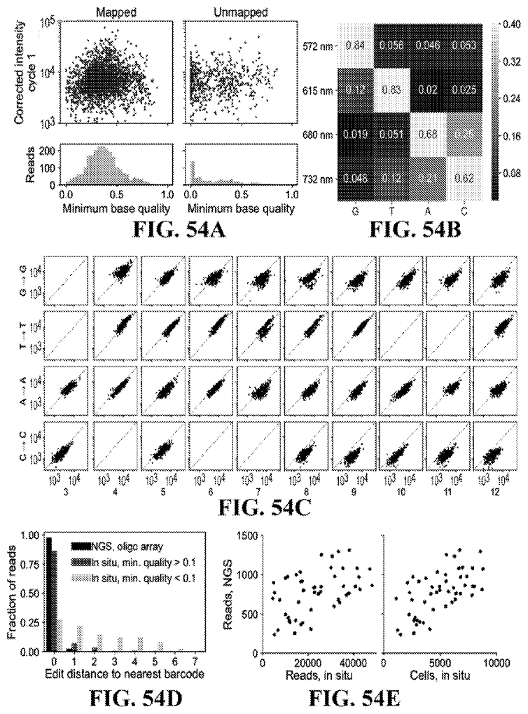

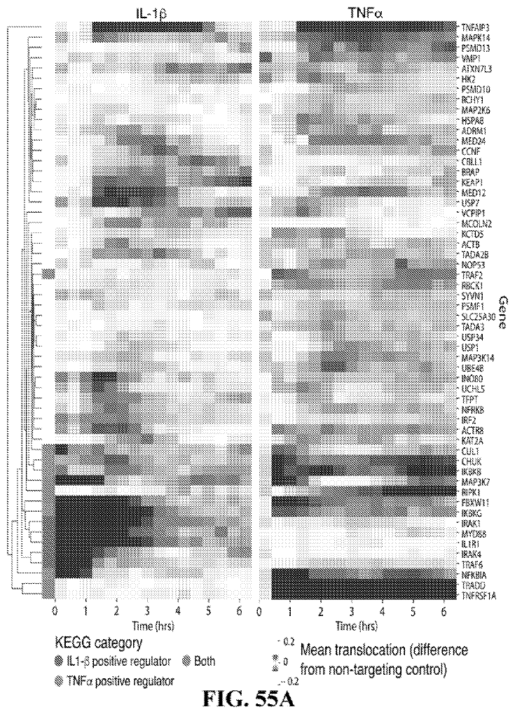

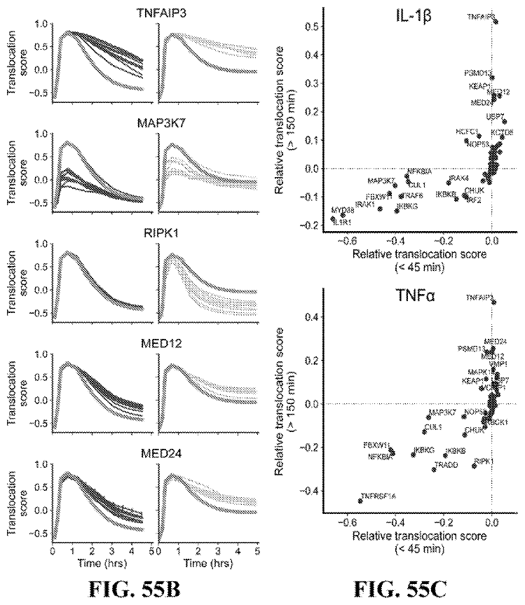

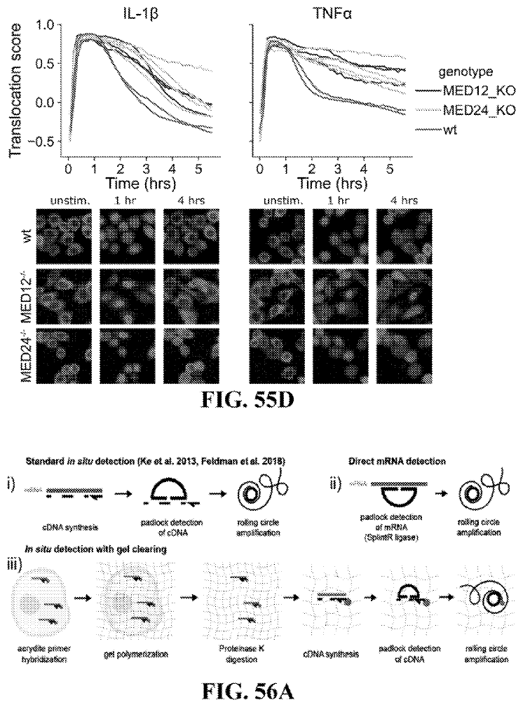

The results of the pooled screen were confirmed by arrayed CRISPR knockouts of a subset of hits. Specifically, 19 out of 20 top-ranked hits were validated (FIG. 47E). Phenotype strength was well correlated between the primary screening and validation data (Spearman's p=0.87 (IL-1β), p=0.76 (TNFα)), emphasizing the quantitative nature of the primary screen (FIG. 53).

The screen also identified potentially novel pathway members that were validated by arrayed knockouts, including BAP1, HCFC1, and KCTD5. Among the IL-1β-specific screening hits, BAP1 has been previously described to deubiquitinate HCFC1 (Machida et al., 2009), with relevance for controlling metabolism (Bononi et al., 2017), ER-stress signaling (Dai et al., 2017), cell-cycle progression (Misaghi et al., 2009), and viral gene expression (Johnson et al., 1999). However, no involvement in NF-κB signaling has been described to Applicants' knowledge. KCTD5, the loss of which led to defects in both IL-1β and TNFα-induced activation, has been shown to physically interact with CUL3 (Bayón et al., 2008; Bennett et al., 2010) and AAV Rep proteins (Weger et al., 2007). While KCTD5 has been proposed to act as a substrate adapter for CUL3, no connection to NF-κB signaling has been previously reported.

### High-Content Analysis of Morphology Distinguishes Regulators by Function

The high content imaging nature of the screen meant that in addition to the translocation phenotype, Applicants captured a morphological profile for each cell. Applicants hypothesized that loss of genes with related function might induce similar morphological changes, providing additional dimensions to interpret the screening results.

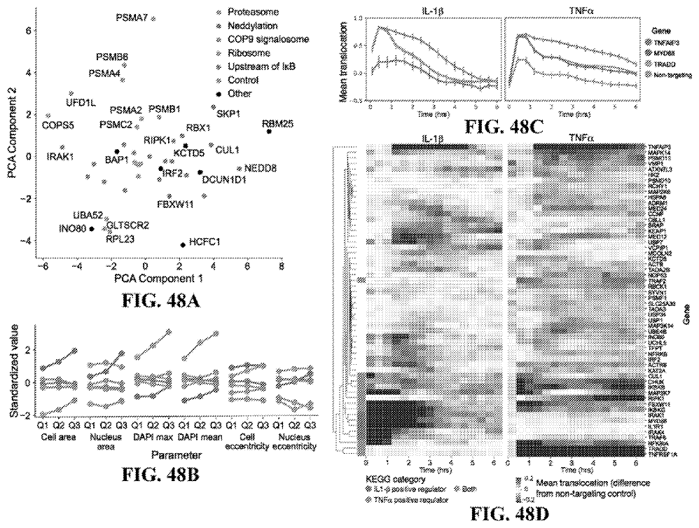

In particular, it was observed for a subset of NF-κB regulators that gene knockouts also induced abnormal nuclear or cellular morphology, and thus re-scored each cell in the primary screen for cell and nuclear morphology, excluding the p65-mNeonGreen signal (STAR Methods). The gene-level (across all guides and cells) distribution of morphological features was summarized by its first, second and third quartiles, producing an 18-dimensional vector for each gene. Applicants performed dimensionality reduction by Principal Components Analysis (PCA) and visualized the first two principal components, finding distinct clusters for regulators downstream of IKK (FIGS. 48A, and 48B). Members of the CUL1-RBX1-SKP1-FBXW11 complex and their substrate NEDD8 showed increased cell and nuclear area, with 4 out of 5 genes grouped together by PCA. Intriguingly, neddylation-related genes had a similar morphological phenotype to RBM25, a splicing factor known to be upregulated by interferon stimulation but not previously linked to NF-κB activation. The top 6 proteasome subunits from the primary screen (highest rank 17, lowest 35) grouped together and showed a distinct morphological profile consisting of rounded cells (low eccentricity) with enlarged nuclei. UFD1L, a member of the VCP/p97-UFD1L-NPL4 complex that mediates post-ubiquitination degradation of IκBα, shared a similar profile to COPS5, which interacts directly with VCP/p97 as part of the COPS signalosome (Cayli et al., 2009; Li et al., 2014). Disruption of the chromatin remodeler INO80, as well as NOP53, GLTSCR2, and UBA52, genes with roles in ribosome biogenesis, showed a decrease in cell and nuclear area. Overall, a stronger morphological signature was observed for downstream regulators, in particular ubiquitin-degradation components, which may reflect broader roles of these genes in cell homeostasis. The ability to group screen hits into functional categories based on morphological changes is a key benefit of image-based screening, which could be further enhanced by staining additional cellular markers to extract more information from each cell.

### Live-Cell Imaging Defines the Kinetics of p65 Activation and Relaxation

Activation of NF-κB involves a cascade of signaling events and feedback loops whose kinetics define the dynamic response of the pathway. As the optical pooled perturbation screening platform can be combined with live-cell imaging, Applicants screened a CROP-seq library of sgRNAs targeting the same gene set for variability in the timing of p65 activation and relaxation (˜6 sgRNAs/gene). Cells were stimulated with either IL-1β or TNFα and imaged at 23 minute intervals up to 6 hours post-stimulation. Nuclei were tracked using a cell-permeable DNA stain and p65-mNeonGreen nuclear translocation was assessed at each timepoint. Following live-cell analysis, cells were fixed and the perturbation in each cell was read out by in situ sequencing.

The live-cell screen closely replicated the initial screen, with 15 out of 20 top positive regulators shared when the live cell analysis was restricted to a single matched time point (STAR Methods). Hierarchical clustering of mean translocation time profiles revealed distinct populations of positive and negative regulators (FIG. 48C, 48D). To quantify changes in translocation kinetics, Applicants defined a cumulative defect for each gene, which was calculated by first integrating over time the difference between each cell's translocation score and the mean translocation score of non-targeting controls. Then, for each gene, the distribution of cell-level cumulative defects was tested for statistically significant deviations from the non-targeting control population (STAR Methods). Applicants identified key negative regulators of the pathway, including TNFAIP3 (an NF-κB target gene that provides negative feedback by deubiquitinating multiple upstream signaling components, Chen 2005), KEAP1 (a ubiquitin ligase involved in degradation of IKKβ) (Lee et al., 2009) and USP7 (a deubiquitinase that slows ubiquitination and proteasomal degradation of NF-κB) (Colleran et al., 2013). In addition, Applicants discovered a group of regulators whose knockout yielded a delayed translocation defect, likely resulting from diminished translocation and/or accelerated relaxation of p65 translocation (e.g., MAP3K17, USP1).

Next, Applicants performed a pooled validation screen for 65 genes that showed a kinetic phenotype (10 sgRNAs/gene). By optimizing conditions for live-cell imaging and tracking, Applicants were able to link time-resolved p65 translocation data to perturbation barcodes from 3,000,000 cells. At this high level of sampling, guides for a given gene showed excellent concordance, with validated hits from the primary screen clearly distinguished from false positives, and clearly distinguished genes with positive or negative effects on p65 translocation from false positives. For example, INO80, RIPK1, KCTD5 and HCFC1 were all detected as weak positive regulators in the initial static screen. While the kinetic validation screen confirmed the role of INO80, RIPK1 showed a much stronger translocation defect at later timepoints while KCTD5 and HCFC1 were seen to negatively regulate p65.

The validation screen also provided strong evidence for MED12, MED24 and VMP1 as novel negative regulators of p65 activity. Clonal knockouts for these genes confirmed their altered translocation kinetics relative to nontargeting guides. In some cases (e.g., MED24), mutant phenotypes were poorly captured by heterozygous knockouts, underscoring that the primary screen was fairly sensitive despite indel heterogeneity. Applicants further investigated the role of these genes in NF-κB signaling by performing RNA-seq on clonal knockout lines. The ability to screen p65 translocation kinetics adds a rich temporal dimension to the profiling of gene knockouts, permitting direct measurement of response onset and duration in addition to strength, directionality (positive/negative) and cytokine specificity.

### Choice of Vector Backbone for Pooled Optical Screens

In this example, Applicants describe two different strategies for CRISPR-based optical screens. The original LentiGuide-BC strategy used an sgRNA and associated barcode separated by a long (˜2 kb) intervening sequence. Applicants and others have noted that such designs can result in a substantial degree of barcode swapping that diminishes the statistical power of screens, see, e.g., Hill, Nature Methods 2018 April; 15(4): 271-274, likely due to homologous recombination during lentiviral integration. Applicants addressed this problem by co-packaging the plasmid library with a non-homologous carrier plasmid (pR_LG), which eliminated barcode swaps but also reduced the functional viral titer. As a second strategy, Applicants used the CROP-seq approach in which the sgRNA is duplicated onto a Pol II transcript that can serve as a template for reverse transcription and in situ detection. In this approach, lentiviral recombination is not an issue and thus the effective viral titers can be much higher (˜100×).

After the oligos used for in situ amplification of the CROP-seq vector were optimized, both strategies produced in situ sequencing spots that could readily be detected at 10× magnification and yielded similar screening throughput. In addition to the improvement in titer, the CROP-seq backbone requires only one cloning step to generate sgRNA libraries (versus two steps for LentiGuide-BC). Although the CROPseq screens in this manuscript use an earlier version of the sgRNA scaffold sequence, Applicants have recently optimized padlock detection probes for a modified scaffold that has been shown to improve CRISPR efficiency. Dang et al., Genome Biology (2015) 16:280, DOI: 10.1186/s13059-015-0846-3.

For these reasons, the CROP-seq backbone is recommended for most CRISPR-based optical screens. For applications where an associated barcode is advantageous or necessary, such as barcoded ORF libraries or dual-sgRNA vector backbones, the LentiGuide-BC flanking sequences may be useful for in situ detection.

Promoter choice and selection strategy also affect the abundance of expressed barcodes In initial screens, Applicants used a CMV promoter that resulted in a bimodal distribution of barcode transcripts in HeLa cells. Comparatively, an EF1a promoter yielded a more uniform distribution in HeLa cells and is a strong promoter in many cell types. The expression of sequence tags can be further boosted by using a more stringent antibiotic selection such as puromycin or zeocin (Takanaka et al) or by FACS-sorting a co-expressed fluorescent marker.

Considerations when Choosing a Cell Line for Screening

When choosing a cell line model for a pooled optical screen, a number of factors must be considered, including the fraction of cells with accurately mapped perturbations, Cas9 efficiency (for CRISPR screens) and suitability for the biological process of interest. Applicants have already demonstrated in situ detection in a panel of cancer cells, including adherent and suspension lines, as well as primary neurons. Additionally, the maximum feasible cell density is an important consideration as it directly impacts screening throughput (i.e., number of cells imaged per unit time and cost per cell).

The perturbation mapping rate, which depends on the number of spots per cell and their brightness, is affected by barcode expression as well as in situ amplification efficiency. Expression level may be tuned by varying promoter choice or selection strategy (See, section on choice of vector) while in situ amplification is likely affected by biophysical properties of the cell after fixation (e.g., molecular constituents and organization). Sequence tag amplification may be optimized by altering the in situ processing (enzyme and dNTP concentrations, buffer components or fixation conditions). The protocol described herein achieves high yield from a number of cell lines and is a recommended starting point.

For CRISPR knockout and CRISPRi/a screens, there are well-described assays to validate Cas9 performance in the target cell line (J Joung, et al., Nat. Protoc. 2017 DOI:10.1038/nprot.2017.016; M. A. Horlbeck, et al., eLife 2016; 5:e19760, DOI:10.7554/eLife.19760). Screens may be performed in a cell line with heterogeneous Cas9 expression; however, flow-enrichment or clonal isolation (as used in this work) may boost CRISPR efficiency and improve statistical power. For knockout screens, indel sequencing provides a quantitative measure of Cas9 performance that can be easily multiplexed across multiple cell lines or clones. For CRISPR knockout screens, the frameshift reporter screen outlined in the main text is a convenient all-in-one test for Cas9 efficiency and perturbation readout in a target cell line/clone; analogous fluorescent reporter screens may be used to validate CRISPRi/a modalities.

Certain cell lines will introduce additional complexities to the screening workflow. Applicants have successfully demonstrated in situ sequencing in suspension cell lines by coating glass substrates with various proteins (PLL, fibronectin, collagen) to facilitate attachment. Neuronal cell types can be challenging to segment accurately, but perturbation assignment can be aided by using low cell densities and restricting attention to barcodes in the soma and nucleus.

Image Analysis Pipeline for Cell Features and Sequencing-by-Synthesis

Phenotype and sequencing-by-synthesis images were acquired on a Nikon Ti-E microscope using Micromanager software. Coarse hardware alignment was performed by using nuclear masks to calibrate the plate position between cycles. Fine alignment was achieved using cross-correlation of nuclei images to determine pixel shifts. Nuclei were then detected using local thresholding and watershed-based segmentation. Cells were segmented using local thresholding of cytoplasmic background and assignment of pixels to the nearest nucleus by the fast-marching method.

Raw sequencing data was processed by applying a Laplacian-of-Gaussian filter (kernel width σ=1 pixel) to subtract low-frequency background and enhance SBS spots. Peaks were detected by calculating the standard deviation in corrected intensity (for each channel) at each pixel over all sequencing cycles, averaging over color channels, and finding local maxima in this standard deviation image. The base intensity at each cycle was defined as the maximum value in a 3×3 pixel window centered on the read. This data was then extracted into a “base table” that summarized the four-color intensities across all cycles for each read along with the read location (x and y positions within each tile).

In order to call bases, a spectral correction for optical cross-talk and intensity differences between color channels was applied as a linear transformation of the LoG-transformed intensity values. The transformation matrix were determined automatically on a tile-by-tile basis to account for local differences in signal intensity and background. Next, basecalling was performed by choosing a base for each cycle according to the channel with the maximum corrected intensity, and a per-base quality score was defined as the ratio of intensity for the maximum channel to total intensity for all channels. This output (barcode calls, quality scores, read locations and cell identities) was saved into a “read table.” Finally, a “cell table” was assembled by noting the top two barcodes for each cell along with their abundances. Cellular phenotypes were analyzed by extracting image features for each cell into a “feature table.” Frameshift reporter and NF-κB translocation phenotypes were quantified by calculating pixel-wise correlations between the nuclear DAPI channel and 488 channel (HA stain or p65-mNeonGreen, respectively).

### Discussion

Pooled optical screens enable systematic analysis of the genetic components underpinning a wide range of spatially and temporally defined phenotypes, including subcellular localization, live-cell dynamics and high-content morphological profiling. Applicants demonstrated a pooled CRISPR loss-of-function screen for regulators of p65 translocation in 3 million cells over 3 days, recovering nearly all major known regulators of TNFα- and IL-1β-stimulated NF-κB activation and discovering potentially novel regulators that were confirmed by arrayed knockouts. High-content screening of cell and nuclear morphology and time-domain analysis of p65 translocation further grouped regulators into functional categories. The in situ sequencing framework is compatible with any perturbation that can be identified by a short expressed sequence, and readily scales to millions of cells, opening the door to genome-scale optical screens of functionally relevant phenotypes in a single sample.

The use of in situ sequencing to read out perturbations has several advantages over alternative approaches. Amplification by RCA enables fast in situ sequencing at low magnification (10×), greatly increasing throughput. The 12-nt sequences used here can robustly distinguish >80,000 perturbations, sufficient to encode genome-scale perturbation libraries. Existing CRISPR sgRNA libraries can be read out directly (using the CROP-seq vector) while other perturbations (e.g., ORFs, non-coding sequences) can be paired with short barcodes (Melnikov et al., 2012; Yang et al., 2011). By comparison, reported methods for highly multiplexed FISH require higher imaging magnification (60×) and require barcodes longer than 200 bp, precluding cost-effective direct oligo array synthesis. Epitope-based protein barcodes are a promising method for enzyme-free decoding of pooled elements, but are currently limited in scale to −100 barcodes (Wroblewska et al., 2018).

Despite their rich phenotypic information, imaging assays have been underused for screening applications due to the substantial cost, labor and expertise required to work with arrayed perturbation reagents. In recent years, there have been many pooled genome-scale CRISPR screens based on enriching cells via fitness or reporter fluorescence in various model systems, but only one large-scale (2,281 sgRNA) CRISPR imaging screen (de Groot et al., 2018). Pooling dramatically reduces the cost and labor required, making genome-scale screens with image-based phenotyping accessible to most laboratories.

Pooled optical screening serves as an important complement to pooled single-cell molecular profiling, which also provides high-content data but is costly at large scales and is not yet able to deliver dynamic or spatial information. The lentiGuide-BC and CROP-seq libraries described in this study both generate mRNA carrying 3′ barcodes, so the same libraries of perturbed cells can potentially be screened in situ and by single cell RNA-seq. This approach could assist in establishing functional relationships between molecular profiles and higher order cellular phenotypes (e.g., morphology, motility, electrical depolarization, cell-to-cell interactions).

The disclosed approach is broadly applicable across many settings. Identification of multiple perturbations within the same cell were demonstrated, providing a straightforward route to study higher order genetic interactions. The potential to integrate optical screening with high-dimensional morphological profiling and in situ multiplexed gene expression analysis (Chen et al., 2015; Ke et al., 2013; Lee et al., 2014; Lubeck et al., 2014; Wang et al., 2018) raises the prospect of learning phenotypes from high-content data rather than pre-specifying phenotypes of interest. Libraries of endogenous or engineered protein variants could be screened for effects on cell structure or other optically-defined phenotypes. Pooled optical screening in a 2D or 3D co-culture system could be used to analyze non-cell autonomous phenotypes based on physical contact (e.g., formation of adhesion complexes, direct contact signaling, neurotransmission). Existing protocols for in situ sequencing in tissue samples (Ke et al., 2013; Wang et al., 2018) highlight the exciting possibility of perturbing cells in vivo and measuring the resulting phenotypes within the native spatial context.

Tissue Culture

HEK293FT cells (ATCC CRL-1573) were cultured in DMEM with sodium pyruvate and GlutaMAX (Life 10569044) supplemented with heat-inactivated fetal bovine serum (Seradigm 97068-085) and 100 U/mL penicillin-streptomycin (Life Technologies 15140163). All HeLa cell lines were cultured in the same media with serum substituted for 10% tetracycline-screened fetal bovine serum (Hyclone SH30070.03T). All other cell lines (A549, HCT116, HT1080, A375) were cultured in the same media with 10% heat-inactivated fetal bovine serum (Sigma F4135).

Parental HeLa-TetR-Cas9 cells were a gift from lain Cheeseman. In order to select an optimal clone for further experiments, single cells were sorted into a 96-well plate (Sony SH800), clonally expanded, and screened for Cas9 activity after 8 days with and without 1 μg/mL doxycycline induction. Cas9 activity was assessed by transducing each clone with pXPR_011 (Addgene #59702), a reporter vector expressing GFP and an sgRNA targeting GFP, and using FACS to read out efficiency of protein knockdown. Additionally, gene editing was directly assessed by transduced HeLa-TetR-Cas9 clones with a guide targeting TFRC. Genomic DNA was extracted from both uninduced and induced clones by resuspending in cell lysis buffer (10 mM Tris pH 7.5, 1 mM CaCl2, 3 mM MgCl2, 1 mM EDTA, 1% Triton X-100, and 0.2 mg/mL Proteinase K), and heating for 10 minutes at 65° C. and 15 minutes at 95° C. The guide target region was amplified by PCR and sequenced on an Illumina MiniSeq. The best clones showed efficient indel generation (≥97%) in the presence of doxycycline and minimal cutting (≤2%) in its absence.

In preparation for in situ analysis, cells were seeded onto glass-bottom plates (6-well: Cellvis P06-1.5H-N, 24-well: Greiner Bio-one 662892, 96-well: Greiner Bio-one 655892) at a density of 50,000 cells/cm2 and incubated for 2 days to permit proper cell attachment, spreading, and colony formation.

### Lentivirus Production

HEK293FT cells were seeded into 15-cm plates or multi-well plates at a density of 100,000 cells/cm2. After 20 hours, cells were transfected with pMD2.G (Addgene #12259), psPAX2 (Addgene #12260), and a lentiviral transfer plasmid (2:3:4 ratio by mass) using Lipofectamine 3000 (Thermo Fisher L3000015). Media was exchanged after 4 hours and supplemented with 2 mM caffeine 20 hours post-transfection to increase viral titer. Viral supernatant was harvested 48 hours after transfection and filtered through 0.45 μm PVDF filters (Millipore SLHVR04NL).

### Lentiviral Transduction

Cells were transduced by adding viral supernatant supplemented with polybrene (8 μg/mL) and centrifuging at 1000 g for 2 hours at 33° C. At 5 hours post-infection, media was exchanged. At 24 hours post-infection, cells were passaged into media containing selection antibiotic at the following concentrations: 1 μg/mL puromycin (ThermoFisher A1113802), 300 μg/mL hygromycin (Invivogen ant-hg-1), 30 μg/mL blasticidin (ThermoFisher A1113903), and 300 μg/mL zeocin (ThermoFisher R25001).

For lentiviral transduction of lentiGuide-BC libraries, a carrier plasmid was utilized to minimize recombination between distant genetic elements (e.g., sgRNA and associated barcode). Libraries were packaged following the above protocol, with the library transfer plasmid diluted in integration-deficient carrier vector pR_LG (1:10 mass ratio of library to carrier, Addgene #112895) (Feldman et al., 2018).

TABLE 12
Screening throughput summarizes the scale and throughput of the NF-κB screen
performed, as well as two hypothetical screens using either low-density cell culture
or a genome-scale perturbation library, assuming a cell mapping rate of 80% (typical for EF1a
in HeLa cells, FIG. 45E) and 600 cells screened per perturbation. The key figure determining
both throughput and cost is the total surface area processed, which affects the volume of
reagents used and the time per sequencing cycle. The total readout time is
substantially reduced by using a two-color chemistry and optimized microscope hardware.

| | | | | | | | Total |
|---|---|---|---|---|---|---|---|
| | | | | | | | readout |
| | | | | | | Total | time, |
| | | Cell | Sequencing | | Reagent | readout | optimized |
| | Cells | density | cycle | # of | cost | time | hardware |
| | (Mapped) | (cells/cm²) | (hours) | cycles | (direct) | (days) | (days) |
| 4,000 | 2,400,000 | 175,000 | 3 | 9 | $549 | 2 | 1 |
| perturbation | | | | | | | |
| screen with | | | | | | | |
| high-density | | | | | | | |
| cells (e.g. | | | | | | | |
| HeLa) | | | | | | | |
| 4,000 | 2,400,000 | 25,000 | 16 | 9 | $3,840 | 6 | 1 |
| perturbation | | | | | | | |
| screen with | | | | | | | |
| low-density | | | | | | | |
| cells (e.g., | | | | | | | |
| neurons) | | | | | | | |
| 60,000 | 36,000,000 | 175,000 | 34 | 11 | $8,909 | 16 | 4 |
| perturbation | | | | | | | |
| screen with | | | | | | | |
| high-density | | | | | | | |
| cells | | | | | | | |

TABLE 13
Reagent cost estimate for in situ barcode readout,
Related to Figures 45-48

| | | Cost/ |
|---|---|---|
| Step | Reagent | 6-well plate |
| Reverse transcription | RevertAID H minus RT | $192.42 |
| Reverse transcription | RNaseIn | $55.62 |
| Padlock extension/ligation | RnaseH | $81.60 |
| Padlock extension/ligation | TaqIT | $0.60 |
| Padlock extension/ligation | Ampligase | $97.98 |
| Rolling circle amplification | Φ29 polymerase | $441.00 |
| | Padlock detection | $869.22 |
| Sequencing by synthesis | Illumina MiSeq v2: | $648.00 |
| | incorporation mix, | |
| | cleavage mix, wash buffer | |
| | Total (12 sequencing cycles) | $1,517.22 |

The major reagent costs (direct) are shown. The cost per 6-well plate corresponds to the NF-κB screen in FIG. 47 and the first row of Table 12.

TABLE 14
NF-kB live-cell screen results.

| gene | KEGG | HeLa-mNeon | | HeLa anti-p65 | | A549 anti-p65 | | HCT116 anti-p65 | |
|---|---|---|---|---|---|---|---|---|---|
| symbol | annotation | IL-1B | TNFa | IL-1B | TNFa | IL-1B | TNFa | TNFa | IL-1B |
| MYD88 | IL-1B | 1 | 164 | 2 | 56 | 2 | 373 | 3 | 250 |
| IL1R1 | IL-1B | 2 | 723 | 1 | 388 | 1 | 928 | 4 | 914 |
| IRAK1 | IL-1B | 7 | 634 | 3 | 432 | 8 | 852 | 1 | 527 |
| TRAF6 | IL-1B | 11 | 135 | 8 | 619 | 6 | 907 | 8 | 858 |
| IRAK4 | IL-1B | 13 | 94 | 4 | 774 | 5 | 829 | 7 | 341 |
| TRAF2 | TNFa | 219 | 10 | 884 | 7 | 924 | 25 | 205 | 730 |
| BIRC2 | TNFa | 309 | 31 | 265 | 10 | 322 | 289 | 327 | 192 |
| TNFRSF1A | TNFa | 309 | 2 | 304 | 1 | 386 | 1 | 757 | 6 |
| RIPK1 | TNFa | 424 | 18 | 721 | 70 | 404 | 32 | 722 | 910 |
| TRADD | TNFa | 424 | 4 | 375 | 4 | 449 | 4 | 721 | 2 |
| BIRC3 | TNFa | 872 | 802 | 586 | 603 | 120 | 359 | 103 | 199 |
| TRAF5 | TNFa | 872 | 723 | 505 | 97 | 592 | 141 | 315 | 771 |
| MAP3K7 | both | 3 | 3 | 6 | 2 | 7 | 3 | 5 | 14 |
| IKBKG | both | 4 | 5 | 5 | 3 | 3 | 2 | 2 | 1 |
| NFKBIA | both | 7 | 1 | 10 | 6 | 20 | 10 | 13 | 18 |
| IKBKB | both | 19 | 12 | 12 | 8 | 13 | 9 | 60 | 96 |
| CHUK | both | 20 | 9 | 7 | 5 | 12 | 6 | 6 | 9 |
| TAB1 | both | 219 | 368 | 743 | 471 | 239 | 456 | 676 | 190 |
| TAB2 | both | 872 | 81 | 81 | 20 | 149 | 18 | 68 | 289 |
| CUL1 | degradation | 4 | 6 | 17 | 9 | 10 | 8 | 12 | 658 |
| FBXW11 | degradation | 6 | 8 | 14 | 16 | 11 | 7 | 9 | 4 |
| SKP1 | degradation | 9 | 6 | 662 | 424 | 19 | 13 | 537 | 45 |
| NEDD8 | degradation | 10 | 12 | 440 | 26 | 9 | 5 | 10 | 374 |

Genes were assigned p-values based on comparing the translocation either at 4 minutes post-stimulation (as in initial screen) or from 45 minutes to 345 minutes post-stimulation (STAR Methods)

### Table 15. p65 Translocation Screen in a Panel of Cell Lines

Screen ranks for p65 translocation defects and a summary table for KEGG genes detected across HeLa, A549 and HCT116 cells, color-coded based on FDR threshold for each screen (FDR<10% hits in dark green; FDR<20% genes in light green; screen negatives in white and FDR>20% genes in). In addition to detecting core pathway members, antibody screens in HeLa, A549 and HCT116 cells provided supporting evidence for weaker hits from the initial HeLa p65-mNeon reporter screen, such as RBCK1, DCUND1, UBE2N, and UF1D.

TABLE 15

| | Number of KEGG genes detected for each category | | | | |
|---|---|---|---|---|---|
| Cell type | IL-1β | TNFα | Both | Degradation | All KEGG |
| HeLa p65-mNeon | 5/5 | 5/7 | 5/7 | 4/4 | 19/23 |
| HeLa | 5/5 | 4/7 | 5/7 | 2/4 | 16/23 |
| A549 | 5/5 | 4/7 | 5/7 | 4/4 | 18/23 |
| HCT116 | 5/5 | 2/7 | 3/7 | 1/4 | 11/23 |

TABLE 16
Screen throughput and in situ sequencing metrics
Numerical summary of screens, including number of cells passing a phenotyping filter (e.g. non-mitotic
with reporter expression above threshold) and number of those cells with ≥1 or 2 mapped reads.

| | | | | cells | # of | # of | |
|---|---|---|---|---|---|---|---|
| | | | # cells | passing | mapped | mapped | |
| | | | imaged | phenotyping | cells | cells | SBS |
| experiment | parental cells | vector backbone | (SBS) | filter | (≥1 read)* | (≥2 reads)* | cycles |
| static p65-mNeon | HeLa TetR-Cas9 | LentiGuide- | 8,168,177 | 4.187,465 | 3,675,137 | 1,912,584 | 9 |
| screen (FIG. 4) | p65-mNeon | CMV-BC | | | | | |
| live-cell primary | HeLa TetR-Cas9 | CROPseq-EF1a | 2,976,016 | 1,008,090 | 434,505 | 410,100 | 8 |
| screen (FIG. 5A) | p65-mNeon | | | | | | |
| combinatorial screen | HeLa TetR-Cas9 | CROPseq-EF1a | 1,501,956 | n/a | 1,213.684 | 1,046.119 | 6 |
| (Fig SX) | p65-mNeon | | | | | | |
| live-cell validation | HeLa TetR-Cas9 | CROPseq-EF1a | 8,515,583 | 5,397,357 | 2,595,514 | 2,065,904 | 8 |
| screen (FIG. 58) | p65-mNeon | | | | | | |
| static p65 antibody | HCT116 | CROPseq-EF1a | 8,682,346 | 8,244,263 | 3,090,260 | 623,101 | 9 |
| screen (FIG. SX) | Cas9-blast | | | | | | |
| static p65 antibody | HeLa TetR-Cas9 | CROPseq-EF1a | 3,637,980 | 3,098,658 | 2,186,852 | 958,257 | 8 |
| screen (FIG. SX) | | | | | | | |
| static p65 antibody | HeLa TetR-Cas9 | CROPseq-EF1a | 6,351,907 | 6,183,487 | 4,327,399 | 2,860,277 | 8 |
| screen (FIG. SX) | | | | | | | |
| static p65 antibody | A549 Cas9-blast | CROPseq-EF1a | 4,970,269 | 4,802,058 | 2,372,971 | 1,172,433 | 9 |
| screen (FIG. SX) | | | | | | | |

*mapped cells are a subcategory of cab that pass through the phenotyping filter

TABLE 17
Key Resources Table

| | |
|---|---|
| Endura Electrocompetent Cells | 69242 |
| TNFα | rcyc-htnfa |
| IL-1β | rcyec-hil1b |
| rabbit anti-HA antibody | 3724 |
| rabbit anti-p65 antibody | 8242 |
| goat anti-rabbit IgG Alexa Fluor 488 | 4412 |
| Revertaid H minus RT | EP0452 |
| Ribolock RNase inhibitor | EO0384 |
| RNase H | Y9220L |
| TaqIT DNA polymerase | P7620L |
| Stoffel Fragment | RP810 |
| Ampligase | A3210K |
| Phi29 DNA polymerase | EP0092 |
| MiSeq 500 cycle Nano kit | MS-103-1003 |
| HeLa-TetR-Cas9 | N/A |
| oRT_lentiGuide-BC (for use with | N/A |
| LentiGuide-BC): | |
| G+AC+GT+GT+GC+TT+AC+CCAAA | |
| GG | |
| oRT_CROPseq (for use with CROPseq- | N/A |
| puro and CROPseq-zeo): | |
| G+AC+TA+GC+CT+TA+TT+TTAAC | |
| TTGCTAT | |
| oRT_CROPseq_ov2 (for use with | N/A |
| CROPseq-puro-v2): | |
| oPD_lentiGuide-BC (for use with | N/A |
| LentiGuide-BC): | |
| /5Phos/actggctattcattcgcCTCCTGTTCG | |
| ACAGTCAGCCGCATCTGCGTCTATT | |
| TAGTGGAGCCC | |
| TTGtgttcaatcaacattcc | |
| oPD_CROPseq_old (used with | N/A |
| CROPseq-puro;): | |
| oPD_CROPseq_v2 (for use with XY): | N/A |
| /5Phos/gttttagagctagaaatagcaagCTCCT | |
| GTTCGCCTATCCCTTCCCTATCCT | |
| CTCTCATACAT | |
| CCAACTCACAaaaggacgaaacaccg | |
| oPD_CROPseq_v2 (for use with | N/A |
| CROPseq-puro-v2): | |
| oSBS_lentiGuide-BC (for use with | N/A |
| LentiGuide-BC): | |
| TTCGACAGTCAGCCGCATCTGCGT | |
| CTATTTAGTGG | |
| AGCCCTTGtgttcaatcaacattcc | |
| oSBS_CROPseq_v2 (for use with | N/A |
| CROPseq-puro and CROPseq-zeo): | |
| TTCGCCTATCCCTTCCCTATCCTCT | |
| CTCATACATC | |
| CAACTCACAaaaggacgaaacaccg | |
| oSBS_CROPseq_v2 (for use with | N/A |
| CROPseq-puro-v2): | |
| LentiGuide-BC | N/A |
| LentiGuide-BC-CMV | N/A |
| CROPseq-Guide-Puro | Addgene Plasmid #86708 |
| pR14_p65-mNeonGreen | N/A |
| pL_FR_Hygro | N/A |
| CROPseq-Puro-v2 | N/A |
| pR_LG | Addgene Plasmid #112895 |
| Light source | Sola SE365 FISH |
| Camera | ORCA-Flash 4.0 v3 |
| Objective Lens | CFI Plan Apochromat Lambda |
| | 10X/0.45 |
| DAPI filter set | LED-DAPI-A-NTE-ZERO |
| GFP filter set | GFP-1828A-NTE-ZERO |
| Cy3 filter set (Miseq G) | FF01-534/20-25 |
| | FF552-Di02-25x36 FF01- |
| | 572/28-25 |
| A594 filter set (Miseq T) | FF03-575/25-25 |
| | FF596-Di01-25x36 FF01- |
| | 615/24-25 |
| Cy5 filter set (Miseq A) | FF01-635/18-25 |
| | FF652-Di01-25x36 FF01- |
| | 680/42-25 |
| Cy7 filter set (Miseq C) | FF01-661/20-25 |
| | FF695-DI01-25X36 FF01- |
| | 732/68-25 |
| Scikit-Image | http://scikit-image.org/ |
| Snakemake | https://snakemake.readthedocs.io/ |
| | en/stable/ |
| Sample in situ sequencing data and | https://github.com/blaineylab/ |
| analysis pipeline from data to | OpticalPooledScreens |
| sequencing read table | |

### Library Cell Line Validation

For library transductions, multiplicity of infection (summarized below) was estimated by counting colonies after sparse plating and antibiotic selection. Genomic DNA was also extracted for NGS validation of library representation.

TABLE 18
Library Transductions

| Experiment | Parental cells | Library backbone | MOI (%) |
|---|---|---|---|
| Static p65-mNEON | HeLa TetR-Cas9 | LentiGuide- | <1 |
| screen (Figure 47) | p65-mNeon | CMV-BC* | |
| Live-cell primary | HeLa TetR-Cas9 | CROPseq-EF1a | 10 |
| screen (Figure 48A) | p65-mNeon | | |
| Live-cell validation | HeLa TetR-Cas9 | CROPseq-EF1a | 10 |
| screen. (Figure 48B) | p65-niNeon | | |
| Static p65 antibody | HeLa TetR-Cas9 | CROPseq-EF1a | 8 |
| screen (Figure SX) | | | |
| Static p65 antibody | A549 Cas9-b1ast | CROPseq-EF1a | 5 |
| screen (Figure SX) | | | |
| Static p65 antibody | HCT116 Cas9-blast | CROPseq-EF1a | 20 |
| screen (Figure SX) | | | |

*Co-packaged 1:10 in pR_LG

### Next Generation Sequencing of Libraries

Genomic DNA was extracted using an extraction mix as described above. Barcodes and sgRNAs were amplified by PCR from a minimum of 1e6 genomic equivalents per library using NEBNext 2× Master Mix (initial denaturation 5 minutes at 98° C., followed by 28 cycles of annealing for 10 seconds at 65° C., extension for 25 seconds at 72° C., and denaturation for 20 seconds at 98° C.).

### Library Design and Cloning

A set of 12-nt barcodes was designed by selecting 83,314 barcodes from the set of 16.7 million possible 12-nt sequences by filtering for GC content between 25% and 75%, no more than 4 consecutive repeated bases, and minimum substitution and insertion/deletion edit distance (Levenshtein distance) of 3 between any pair of barcodes. Ensuring a minimum edit distance is useful for detecting and correcting errors, which arise mainly from DNA synthesis and in situ reads with low signal-to-background ratios. The E-CRISP web tool was used to select sgRNA sequences targeting genes of interest. Barcode-sgRNA pairs were randomly assigned and co-synthesized on a 125-nt 90K oligo array (CustomArray/Genscript). Individual libraries were amplified from the oligo pool by dial-out PCR (Schwartz et al., 2012) and cloned into lentiGuide-BC or lentiGuide-BC-CMV (the latter contains the CMV promoter instead of the EF1a promoter) via two steps of Golden Gate assembly using BsmBI restriction sites. Then, the sgRNA scaffold sequence and desired resistance cassette were inserted using BbsI restriction sites. Libraries were transformed in electrocompetent cells (Lucigen Endura) and grown in liquid culture for 18 hours at 30° C. before extracting plasmid DNA. The sgRNA-barcode association was validated by Sanger sequencing individual colonies from the final library.

### Padlock-Based RNA Detection

Preparation of targeted RNA amplicons for in situ sequencing was adapted from published protocols with modifications to improve molecular detection efficiency and amplification yield (Ke et al., 2013; Larsson et al., 2010). Cells were fixed with 4% paraformaldehyde (Electron Microscopy Sciences 15714) for 30 minutes, washed with PBS, and permeabilized with 70% ethanol for 30 minutes. Permeabilization solution was carefully exchanged with PBS-T wash buffer (PBS+0.05% Tween-20) to minimize sample dehydration. Reverse transcription mix (1× RevertAid RT buffer, 250 μM dNTPs, 0.2 mg/mL BSA, 1 μM RT primer, 0.8 U/μL Ribolock RNase inhibitor, and 4.8 U/μL RevertAid H minus reverse transcriptase) was added to the sample and incubated for 16 hours at 37° C. Following reverse transcription, cells were washed 5 times with PBS-T and post-fixed with 3% paraformaldehyde and 0.1% glutaraldehyde for 30 minutes at room temperature, then washed with PBS-T 5 times. After this step, cells expressing p65-mNeonGreen were imaged. Samples were thoroughly washed again with PBS-T, incubated in a padlock probe and extension-ligation reaction mix (1× Ampligase buffer, 0.4 U/μL RNase H, 0.2 mg/mL, BSA, 10 nM padlock probe, 0.02 U/μL TaqIT polymerase, 0.5 U/μL Ampligase and 50 nM dNTPs) for 5 minutes at 37° C. and 90 minutes at 45° C., and then washed 2 times with PBS-T. Circularized padlocks were amplified with rolling circle amplification mix (1× Phi29 buffer, 250 μM dNTPs, 0.2 mg/mL BSA, 5% glycerol, and 1 U/μL Phi29 DNA polymerase) at 30° C. for either 3 hours or overnight.

### In Situ Sequencing

Rolling circle amplicons were prepared for sequencing by hybridizing a mix containing sequencing primer oSBS_lentiGuide-BC or oSBS_CROP-seq (1 μM primer in 2×SSC+10% formamide) for 30 minutes at room temperature. Barcodes were read out using sequencing-by-synthesis reagents from the Illumina MiSeq 500 cycle Nano kit (Illumina MS-103-1003). First, samples were washed with incorporation buffer (Nano kit PR2) and incubated for 3 minutes in incorporation mix (Nano kit reagent 1) at 60° C. Samples were then thoroughly washed with PR2 at 60° C. (6 washes for 3 minutes each) and placed in 200 ng/mL DAPI in 2×SSC for fluorescence imaging. Following each imaging cycle, dye terminators were removed by incubation for 6 minutes in Illumina cleavage mix (Nano kit reagent 4) at 60° C., and samples were thoroughly washed with PR2.

### Fluorescence Microscopy

All images were acquired using a Ti-E Eclipse inverted epifluorescence microscope (Nikon) with automated XYZ stage control and hardware autofocus. An LED light engine (Lumencor Sola SE FISH II) was used for fluorescence illumination and all hardware was controlled using Micromanager software (Edelstein et al., 2010). In situ sequencing cycles were imaged using a 10×0.45 NA CFI Plan Apo Lambda objective (Nikon) with the following filters (Semrock) and exposure times for each base: G (excitation 534/20 nm, emission 572/28 nm, dichroic 552 nm, 200 ms); T (excitation 575/25 nm, emission 615/24 nm, dichroic 596 nm, 200 ms); A (excitation 635/18 nm, emission 680/42 nm, dichroic 652 nm, 200 ms); C (excitation 661/20 nm, emission 732/68 nm, dichroic 695 nm, 800 ms).

### Image Analysis

Images of cell phenotype and in situ sequencing of perturbations were aligned using cross-correlation of DAPI-stained nuclei. Nuclei were detected using local thresholding and watershed-based segmentation. Cells were typically segmented using local thresholding of cytoplasmic background and assignment of pixels to the nearest nucleus by the fast-marching method. Frameshift reporter and NF-κB translocation phenotypes were quantified by calculating pixel-wise correlations between the nuclear DAPI channel and 488 channel (HA stain or p65-mNeonGreen, respectively).

Sequencing reads were detected by applying a Laplacian-of-Gaussian linear filter (kernel width σ=1 pixel), calculating the per-pixel standard deviation over sequencing cycles, averaging over color channels, and finding local maxima. The base intensity at each cycle was defined as the maximum value in a 3×3 pixel window centered on the read. A linear transformation was then applied to correct for optical cross-talk and intensity differences between color channels. Finally, each base was called according to the channel with maximum corrected intensity, and a per-base quality score was defined as the ratio of intensity for the maximum channel to total intensity for all channels. The output of the sequencing image analysis was a FASTQ file recording each sequencing read along with the identity of the overlapping cell, quality score per base, and spatial location.

Additional details and source code available at gitbub.com/blaineylab/OpticalPooledScreens.

### Frameshift Reporter Screen

HeLa-TetR-Cas9 cells were stably transduced at MOI>2 with pL_FR_Hygro and selected with hygromycin for 7 days to generate the HeLa-TetR-Cas9-FR cell line. Cells transduced with the pL_FR_Hygro lentiviral vector express an open reading frame consisting of a 50-nt frameshift reporter target sequence, followed by an H2B histone coding sequence with C-terminus HA epitope tag (+1 frameshift), followed by a second H2B sequence with C-terminus myc tag (+0 frameshift) and hygro antibiotic resistance cassette (+0 frameshift). The H2B-HA, H2B-myc, and hygromycin resistance sequences are preceded by self-cleaving 2A peptides in the same reading frame. Before generation of Cas9-mediated indel mutations, cells express the coding sequences with +0 frameshift. Subsequent activation of the reporter by co-expression of Cas9 and a targeting sgRNA leads to mutations in the target sequence, which may alter the downstream reading frame. A frameshift of +1 leads to expression of the H2B-HA protein, which can be visualized by immunofluorescence and detected by microscopy or flow cytometry. Integration of multiple copies of reporter per cell increases the likelihood of generating a +1 frameshift in at least one copy.

HeLa-TetR-Cas9-FR cells were used to screen targeting and control sgRNAs. A barcoded sgRNA perturbation library with 972 barcodes, each encoding one of 5 targeting or 5 control sgRNAs, was synthesized and cloned into lentiGuide-BC-CMV. This library was transduced into HeLa-TetR-Cas9-FR cells at MOI<0.05 in three replicates, which were independently cultured and screened. Following 4 days of puromycin selection, cells were collected to validate library representation by NGS. Cas9 expression was induced by supplementing the culture media with 1 μg/mL doxycycline for 6 days. Cells were then split for screening either via in situ sequencing or by FACS.

Frameshift reporter screens in U2-OS, A375, HT1080, HCT116 and HEK293T cell types were performed by first transducing these cells with lentiCas9-blast (Addgene #52962) and selecting with blasticidin for 4 days. Cas9-expressing cells were then transduced at MOI>2 with pL_FR_Hygro and selected with hygromycin for 7 days to generate reporter lines. These reporter lines were transduced with a CROPseq library of guides consisting of 5 sgRNAs complementary to the frameshift reporter target region and 5 non-targeting sgRNAs. Cell libraries were selected with puromycin for 4 days and cells were seeded onto glass-bottom dishes 8 days after transduction.

For in situ screening, 500,000 cells were seeded into each well of a glass-bottom 6-well plate (CellVis). After two days of culture, in situ padlock detection and sequencing were carried out as above, with the modification that prior to sequencing-by-synthesis, cells were immunostained to detect frameshift reporter activation by blocking and permeabilizing with 3% BSA+0.5% Triton X-100 for 5 minutes, incubating in rabbit anti-HA (1:1000 dilution in 3% BSA) for 30 minutes, washing with PBS-T and incubating with goat anti-rabbit F(ab′)2 fragment Alexa 488 (CST 4412S, 1:1000 dilution in 3% BSA) for 30 minutes. Samples were changed into imaging buffer (200 ng/mL DAPI in 2×SSC) and phenotype images were acquired.

FACS screening was carried out by fixing cells with 4% PFA, permeabilizing with 70% ice-cold ethanol, and immunostaining with the same anti-HA primary and secondary antibodies and dilutions used for in situ analysis. Cells were sorted into HA+ and HA-populations (Sony SH800) and genomically integrated perturbations were sequenced as described above. The enrichment for each barcoded perturbation was defined as the ratio of normalized read counts.

To estimate the rate at which cells are assigned an incorrect perturbation barcode, Applicants first assumed all HA+ cells mapped to a non-targeting control sgRNA (4.7%) were false positive events due to incorrect barcode assignment (supported by the very low false positive rate (<0.001%) of the frameshift reporter itself, measured for a single perturbation in arrayed format). However, as incorrect barcode assignments were equally likely to map an HA+ cell to a targeting or control sgRNA, Applicants estimated the misidentification rate to be twice as large, or 9.4%.

### NF-κB Static Screen

HeLa-TetR-Cas9 cells were transduced with pR14_p65-mNeonGreen, a C-terminal fusion of p65 with a bright monomeric green fluorescent protein (Allele Biotechnology). Fluorescent cells were sorted by FACS (Sony SH800) and re-sorted to select for cells with stable expression. This reporter cell line was further transduced with a 4,063-barcode sgRNA library (962 genes targeted and 952 detected, 866 barcodes assigned to non-targeting controls) in lentiGuide-BC-CMV. Cells were selected with puromycin for 4 days and library representation was validated by NGS.

Cas9 expression was induced with 1 μg/mL doxycycline and cells were seeded onto 6-well cover glass-bottom plates 2 days prior to translocation experiments. The total time between Cas9 induction and performing the NF-κB activation assay was 7 days. Cells were stimulated with either 30 ng/mL TNFα or 30 ng/mL IL-1β (Invivogen) for 40 minutes prior to fixation with 4% paraformaldehyde for 30 minutes and initiation of the in situ sequencing protocol. Translocation phenotypes were recorded after the post-fixation step by exchanging for imaging buffer (2×SSC+200 ng/mL DAPI) and imaging the nuclear DAPI stain and p65-mNeonGreen. After phenotyping, the remainder of the in situ sequencing protocol (gap-fill and rolling circle amplification) and 12 bases of sequencing-by-synthesis were completed.

HeLa Cas9-blast, A549 Cas9-blast and HCT116 Cas9-blast cells were transduced with a 1052 sgRNA library (952 genes targeted, 100 non-targeting controls) in CROPseq-puro. Cells were selected for 4 days and seeded onto 6-well plates 2 days prior to translocation experiments. The total time between library transduction and the screen was 7-14 days. Cells were stimulated with TNFα or IL-1β (30 ng/mL for HCT116, 3 ng/mL for HeLa and A549) for 40 minutes prior to fixation with 4% paraformaldehyde for 30 minutes and initiation of the in situ sequencing protocol. Cells were stained after the post-fixation step by incubating for 1 hour with a rabbit antibody against p65 (CST 8242S, 1:400 dilution in 3% BSA), washing with PBS-T, then incubating with goat anti-rabbit F(ab′)2 fragment Alexa 488 (CST 4412S, 1:1000 dilution in 3% BSA) for 45 minutes and washing with PBS-T before proceeding to the gap-fill step. After RCA, cells were exchanged into imaging buffer (2×SSC+200 ng/mL DAPI) and imaged with DAPI and 488 (p65) filters. After phenotyping, sequencing primer was hybridized and 10 bases of sequencing-by-synthesis were acquired.

Nuclei of individual cells were segmented by thresholding background-subtracted DAPI signal and separating the resulting regions using the watershed method. Cells with at least one read exactly matching a library barcode were retained for analysis. In order to remove mitotic cells and cells with abnormally high or low reporter expression, cells were further filtered based on nuclear area, maximum DAPI signal, and mean p65-mNeonGreen signal. Pixel-wise DAPI-mNeonGreen correlation within the segmented nuclear region, described above, was used to define the translocation score for each cell as it most clearly separated perturbations against known NF-κB genes from non-targeting controls. The phenotypic effects of perturbations targeting known NF-κB genes ranged from a large increase in fully untranslocated cells (e.g., MAP3K7) to more subtle negative shifts in the distribution of scores (e.g., IKBKB).

To capture a broad range of effect size, Applicants calculated an sgRNA translocation defect in a given replicate by computing the difference in translocation score distribution compared to non-targeting controls (the shaded area in FIGS. 47C and 47E). Applicants found this metric performed better at separating known genes from controls than the often-used Kolmogorov-Smirnov distance. Applicants defined the gene translocation defect as the second-largest sgRNA translocation defect for sgRNAs targeting that gene. This statistic helps reduce the false positive rate due to clonal effects (integration of an sgRNA into a cell that is defective in translocation) which are independent among sgRNAs and screen replicates, as well as false negatives due to inefficient sgRNAs.

A permutation test was used to calculate p-values for the gene translocation defects. Random subsets of sgRNA translocation defects were sampled from non-targeting controls to build a null distribution (3 sgRNAs per replicate, repeated 100,000 times). The cumulative null distribution was used to determine p-values for the gene translocation defects. Hits at an estimated FDR<10% and <20% were identified using the Benjamini-Hochberg procedure. KEGG-annotated genes were defined as members of KEGG pathway HSA04064 (NF-kappa B signaling pathway) between IL-1β or TNFα and p65/p50.

### NF-κB Live-Cell Screen

HeLa-TetR-Cas9 stably expressing the p65-mNeonGreen reporter were further transduced with a CROP-seq library of 5638 sgRNAs targeting the same set of 952 genes as well as 100 non-targeting sgRNAs (present at 5% of total abundance). Cells were selected with puromycin for 4 days and library representation was validated by NGS.

Cas9 expression was induced for 7 days with 1 μg/mL doxycycline and cells were seeded onto 6-well cover glass-bottom plates 2 days prior to translocation experiments. The total time between the start of Cas9 induction and performing the NF-κB live-cell assay was 14 days. Prior to the experiment, culture media was exchanged for imaging media: 0.1 ng/mL Hoechst 33342 in phenol red-free DMEM with HEPES and L-glutamine (Life 21063029), supplemented with 10% FBS and 100 U/mL penicillin-streptomycin. Cells were returned to the incubator for 2 hours, then stimulated with either 30 ng/mL TNFα or 30 ng/mL IL-1β and immediately loaded onto an automated live-cell microscope with environmental control (Zeiss CellDiscoverer 7). Images of the Hoechst nuclear stain and p65-mNeonGreen were acquired at 5× magnification at 23 minute intervals over 6 hours, tiling two wells of a 6-well plate (one well per cytokine). Immediately after stopping live-cell imaging, cells were fixed and the in situ sequencing protocol was carried out as above.

Image analysis was conducted as for the initial NF-κB screen, with additional pre-processing steps to (a) track cells through the live-cell timecourse, and (b) align the final timepoint of live-cell with the first cycle of sequencing-by-synthesis. For each cell, the translocation score at each timepoint was subtracted from a baseline translocation score, interpolated in time from control cells imaged in the same well. For each gene, an integrated translocation score was calculated by integrating each cell's baseline-subtracted translocation scores from 45 to 345 minutes post-stimulation. Statistical significance for deviation in the integrated translocation score between perturbed and control cells was quantified using the non-parametric Mann-Whitney U test.

### NF-κB Arrayed Validation

Top-ranking genes from the primary pooled screen were validated with individual sgRNAs. For each gene, 2 or 3 sgRNAs were tested, including at least one sgRNA not used in the primary screen. HeLa-TetR-Cas9 cells expressing p65-mNeonGreen were prepared and assayed as in the pooled screen, except that cells were transduced with the lentiGuide-Puro sgRNA expression vector (Addgene #52963), and the translocation assay was carried out in 96-well cover glass plates. Image acquisition and data analysis were performed with the same hardware and software settings as in the pooled screen.

The translocation defect for each sgRNA and cytokine translocation was assessed by computing the difference in translocation score distribution compared to the average of at least 3 non-targeting control sgRNAs assayed on the same plate. For each cytokine, the translocation defects were standardized using the mean and standard deviation of the translocation defects for non-targeting control guides. These standardized values were averaged over replicate sgRNAs for a given gene and cytokine pair to obtain validation Z-scores.

### Example 8—Pooled Optical Screening in Tissues

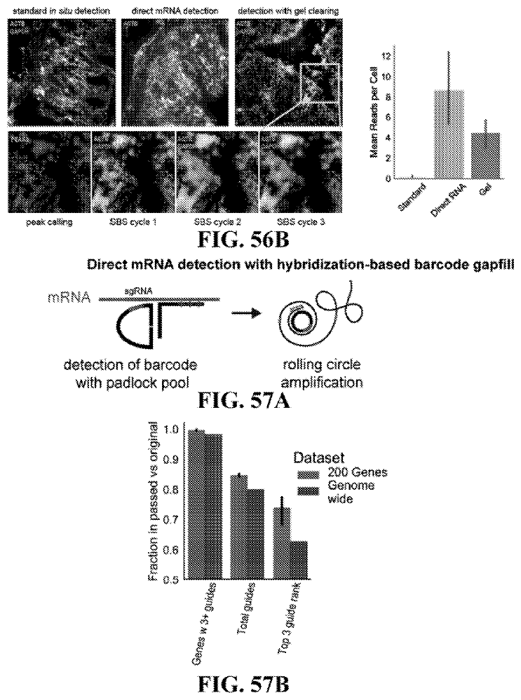

Feasibility testing of in situ detection of target RNAs in tissues using one of three methods (FIG. 56a) have been completed: (i) standard in situ detection (adapted from the Nilsson group) that has performed robustly in cultured cells but did not perform well in tissue sections, (ii) direct mRNA detection to eliminate the inefficient RT step, and (iii) in situ detection with acrydite RT primers covalently linked to an acrylamide gel, paired with proteinase K clearing of the tissue matrix to improve in situ reagent activity. FIG. 1b demonstrates that both modifications of our standard approach achieved significantly improved spot counts compared to the standard approach (FIG. 56ai-) in mouse colon tissue.

### Example 9—Optimization of Screening in Tissues

Optimization of the detection approach described in Example 8 will continue to culminate in the first all-optical in vivo screen enabling linkage of perturbations with in vivo organization of cellular communities for the first time.

The barcode detection optimally suited for tissues will be identified after comparing two approaches: i) direct RNA detection (FIG. 56aii), which will be modified to enable more efficient detection of sgRNAs (FIG. 57a), and ii) the standard in situ method with a gel clearing step (FIG. 56aiii), which already easily accommodates guide detection.

One approach will adapt direct RNA detection with SNAIL probes (Wang et al. 2018) to be compatible with barcode detection by using a padlock pool. This approach will be validated with endogenously expressed transcripts in tissues. Hybridization specificity will be quantified with library of 1000+ guides in cultured cells, and hybridization conditions, including temperature, formamide concentration, time, and may include other blocking reagents such as tRNA, salmon sperm DNA. Quantifying extent of diffusion with mouse/human cell coculture will be performed, including, if required, modifying the protocol to include a postfix step to improve anchoring or linking padlock to acrylamide gel with an acrydite-linked RCA primer. This approach is ideal for efficient barcode detection because: it lacks inefficient RT and gapfill, specificity is maintained via two hybridization events to the mRNA. The approach also enables anchoring of the RCA product to the RNA through LNA bases, including modification of STARMAP protocol, which could remove the requirement for gel formation (as gelation renders the protocol less scalable and consistent). It also requires only a single dsDNA ligation event, which is more efficient than RNA ligation (FIG. 56aii). Further, the approach potentially enables detection of Pol II sgRNA expression in addition to Pol II expression since no hybridization 5′ to the guide is required. However, production of a padlock pool complementary to guides of interest is required. Therefore, validation of a protocol for padlock pool production by reverse transcription of in vitro transcribed RNAs will also be completed.

Increasing efficiency of in situ detection with gel clearing will also be explored. Improving consistency of casting thin gels in tissues and cultured cells (target 90+% success, currently ˜40-50% gels with no visible defects). Casting o thing gels will include improving degassing chamber and titrate acrylamide percentage and testing a variety of initiation strategies to optimize consistency. Improving detection efficiency will be explored by using a TSO to detect Pol III sgRNA expression in addition to Pol II expression in cultured cells. Applicants will also work on quantifying the extent of diffusion in gels with mouse/human cell coculture. The approach to adapting direct RNA detection with SNAIL probes relies on hybridization rather than polymerization for guide detection, which may be less specific. Using an in silico model for FISH probe binding (FIG. 57b), Applicants have shown that, especially for small sets of 200 genes (approximately the upper limit per mouse for an in vivo screen), guides passing filter for specific binding retained 75% of the initial guide efficiency ranking and over 99% of genes retained at least 3 guides per gene, indicating that hybridization is a viable approach for small gene sets. If hybridization specificity is experimentally inferior to in silico predictions, Applicants could improve specificity at the expense of sensitivity through a variety of strategies, for instance by requiring binding and ligation of two probes complementary to the guide of interest.

Next, vigorous validation of the top in situ method selected in a minimum of two tissues of interest for screening will be completed, with performance of pilot in vivo screens. For the two top tissues of interest for screens, several parameters will be optimized. RNA preservation, including possible use of RNAlater, Ribolock, SuperaseIN. Permeabilization conditions will also be optimized, including use of MeOH, EtOH, SDS, MOPS, pepsinHCl. Enzymatic steps will be optimized, including primarily enzyme and dNTP quantities and reaction time/temperature. Tissue digestion, if gelation-based protocol is used, can be optimized for incubation time, concentration, and reagent, for example, proteinase K, collagenase/Liberase.

Imaging conditions will be addressed, including determining magnification required for imaging, if higher than 10× consider techniques to mitigate photodamage: Imaging in IlluminaSCANmix, Trolox, and/or Vitamin E to avoid free radical formation, and not imaging DAPI at every cycle or reducing DAPI UV intensity. Quantify sequencing quality over 6+ cycles using the GFP or frameshift reporter technical validation mouse tissue. Improve multicycle signal to background through washing optimization (e.g. include detergents) and/or electrophoresis washing of molecules bound to gel (similar to CLARITY technique, see protocol method available at Abcam.com); and titration of Illumina incorporation mix.

Increased throughput of tissue imaging will also be addressed. Currently, 12 mm #2 coverslips are being used, but determination of coverslips, and construction of a reliable coverslip adapter for multi-sample imaging over multiple cycles will aid in optimization of tissue imaging throughput.

Finally, Applicants will execute a first full-scale in vivo screen. In order to complete a first full-scale in-vivo screen, steps will include preparing a mouse colony for technical testing in tissues. Steps will include preparing a frameshift reporter mouse, which will be analogous to frameshift reporter Applicants developed for pooled optical screens in cells, as provided herein. Alternatively, Applicants can cross GFP mouse with Cas9 mouse and transduce with GFP-targeting guides vs non-targeting guides. This, however, is less ideal than frameshift reporter due to potential baseline expression variability. Solidify plans for top 2-3 in vivo optical screens of interest and 2 associated tissue types (e.g. lymph node/spleen and tumor). Guide constructs between current Regev lab in vivo Perturb-seq screens and our optical screens in cells (standard CROP-seq vector) are already reconciled. Refining screen ideas and design panels of genes to assay will aid in finalizing the in vivo screen planning.

Performing a pilot in vivo screen (<100 guides), including validation of antibodies and/or padlocks against targets of interest will be performed in planning of the in vivo screen. Refining the computational pipeline for spot detection and expanding the pipeline to phenotypic analyses for screen of interest will include developing robust computational pipeline for analysis of tissue-based optical screens, including improvement of cell segmentation, background subtraction, and spot detection in highly heterogeneous tissue samples. Automation of multicycle sequencing in coverslips will further enable high throughput of tissue samples.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method for screening cells for presence of one or more genetic elements comprising:
a) culturing one or more cells or a cell population in one or more discrete volumes;
b) introducing one or more polynucleotides into the cell or cell population, wherein each polynucleotide comprises nucleic acid sequences encoding a sequence defining one or more optical barcodes and the one or more genetic elements, and wherein a different optical barcode is assigned to each genetic element or a group of the one or more genetic elements, or wherein the genetic element sequence is the optical barcode;
c) incubating the cell or cell population to allow for expression of RNA transcripts comprising the one or more optical barcodes;
d) detecting genomic, genetic, epigenetic, proteomic, and/or phenotypic differences caused by the one or more genetic elements in the cell or cell population; and
e) detecting the optical barcode by an in situ sequencing method to identify the one or more genetic elements present in the cell or cell population.

2. The method of claim 1, wherein the polynucleotide sequence encoding one or more genetic elements comprises or encodes a gene, a modified/damaged/non-natural nucleotide or nucleotide analog, an overexpressed gene, an RNAi based system, a regulatory RNA, a non-coding RNA, an mRNA, a zinc finger nuclease, a transcription activator-like effector nuclease (TALEN), a meganuclease, a computationally designed protein, a computationally designed RNA, or a CRISPR-Cas system.

3. The method of claim 1, wherein introducing one or more polynucleotides into the cell or cell population comprises at least two polynucleotides.

4. The method of claim 1, wherein in step a) the one or more cells or the cell population comprise the same genotype.

5. The method of claim 4, comprising two or more discrete volumes in step a), each discrete volume comprising one or more cells or cell population.

6. The method of claim 2, wherein the polynucleotide sequence encoding one or more genetic elements encodes a CRISP-Cas system.

7. The method of claim 6, wherein the CRISPR-Cas system is a CRISPR-Cas9 or a CRISPR-Cpf1 system.

8. The method of claim 6 or 7, wherein the polynucleotide sequence encodes one or more guide sequences.

9. The method of claim 1 or 2, wherein the one or more genetic elements target genes in a pathway or intracellular network.

10. The method of claim 1 or 2, wherein the one or more genetic elements cause gene knock-down, gene knock-out, gene activation, gene insertion, insertion of a foreign sequence tag, or regulatory element deletion.

11. The method of claim 6, wherein the one or more genetic elements comprise pooled single or combinatorial CRISPR-Cas-based perturbation with a genome-wide library of sgRNAs.

12. The method of any of claims 1-11, wherein step d) comprises determining a phenotypic difference by capturing a microscopic image or time series of microscopic images of the cell or cell population; and correlate the phenotypic difference to the identified one or more genetic elements.

13. The method of any of claims 1-11, wherein step d) comprises measuring differences of DNA, RNA, protein, or post-translational modification, or measuring differences of protein or post translational modification correlated to RNA and/or DNA level(s).

14. The method of any of claims 1-9, wherein step e) further comprises generating a cDNA copy of the RNA transcripts prior to detecting the optical barcode.

15. The method of claim 14, further comprising amplifying the generated cDNA copy prior to detecting the optical barcode.

16. The method of claim 15, wherein the generated cDNA copy is amplified by rolling circle amplification or hybridization chain reaction.

17. The method of any of claims 1-16, wherein the in situ sequencing method is selected from the group consisting of fluorescent in situ RNA sequencing (FISSEQ), in situ mRNA-seq, padlock in situ sequencing, sequencing by ligation, SOLiD® sequencing, and sequencing by synthesis.

18. The method of any of claims 1-17, wherein the RNA transcripts comprising the one or more optical barcodes further comprise a cell localization signal or other sequence ultimately localizing the RNA transcripts to a specific location within the cell.

19. The method of claim 18, wherein the cell localization signal is a nucleus localization signal or a nuclear export/exclusion signal.

20. The method of any of claims 1-17, wherein the RNA transcripts comprising the one or more optical barcodes further comprise a premature termination signal to prevent translation of the RNA transcripts comprising the one or more optical barcodes.

21. The method of any of preceding claims, wherein each optical barcode is about 4 bp to about 32 bp in length.

22. The method of claim 21, wherein each optical barcode is 12 bp.

23. The method of any of the preceding claims, wherein the one or more polynucleotides are introduced to the cell or cell population by a lentiviral or retroviral system.

24. The method of claim 23, wherein the lentiviral or retroviral system has reduced recombination activity, or template switching activity, or multiple integration activity.

25. The method of claim 24, wherein the lentiviral or retroviral system comprises an inhibitor of template switching.

26. The method of claim 24 or 25, wherein the lentiviral or retroviral system comprises a carrier polynucleotide.

27. The method of claim 26, wherein the carrier polynucleotide comprises non-recombinogenic RNA sequences or proteins that are capable of dimerizing with the polynucleotides comprising optical barcodes and genetic elements.

28. The method of claim 24, wherein the reduced recombination or template activity comprises reduced hairpin formation or dimerization through modification, knockdown or knockout of lentiviral or retroviral genomic RNA, or lentiviral or retroviral protein involved in dimerization.

29. The method of claim 28, wherein the modification, knockdown or knockout of the lentiviral or retroviral genomic RNA or lentiviral or retroviral protein comprises modification, knockdown or knockout of nucleocapsid (NC)-protein(s) or RNA for expression thereof or modification, knockdown or knockout of stem-loop I element (SLI) element or modification, knockdown or knockout of genomic RNA whereby U5:AUG pairing is prevented, or modification, knockdown or knockout of a dimer initiation site (DIS).

30. The method of claim 23, wherein the lentiviral or retroviral system comprises the genetic element in the 3′ LTR of the lentiviral genome.

31. The method of any of claims 1-30, wherein the individual discrete volume is a well of a tissue culture plate or slide in a tissue culture plate.

32. The method of any of claims 1-30, wherein the individual discrete volume is a droplet generated on a microfluidic device.

33. The method of any of claims 1-32, wherein the cell or cell population is contained within or isolated from a tissue sample.

34. The method of any of claims 1-32, wherein the cell or cell population is contained within or isolated from a living animal.

35. The method of claim 33, wherein the tissue sample is a biopsy sample from a mammalian subject.

36. The method of claim 35, wherein the mammalian subject is a human subject.

37. The method of claim 35, wherein the biopsy sample is a tumor sample.

38. A system for screening cells for presence of one or more genetic elements, comprising:
a) one or more polynucleotides, wherein each polynucleotide comprises nucleic acid sequences encoding a sequence defining one or more optical barcodes and the one or more genetic elements, and wherein a different optical barcode is assigned to each genetic element or a group of the one or more genetic elements, or wherein the genetic element sequence is the optical barcode, and wherein introduction of the one or more polynucleotides into the cell or cell population results in expression of RNA transcripts comprising the one or more optical barcodes;
b) a first detection system for detecting genomic, genetic, epigenetic, proteomic and/or phenotypic differences caused by the one or more genetic elements in the cell or cell population; and
c) a second detection system for detecting the one or more optical barcodes in the cell or cell population by in situ sequencing.

39. The system of claim 38, further comprising one or more components for generating a cDNA copy of the RNA transcripts prior to detecting the one or more optical barcodes.

40. The system of claim 39, further comprising one or more components for amplifying the generated cDNA copy.

41. The system of any of claims 38-40, wherein the one or more polynucleotides are packaged in a lentiviral or retroviral system.

42. The system of claim 41, wherein the lentiviral or retroviral system has reduced recombination activity, or template switching activity, or multiple integration activity.

43. The system of claim 41, wherein the lentiviral or retroviral system comprises the genetic element in the 3′ LTR of the lentiviral genome.


SEQUENCE LISTING (22 sequences)

SEQ ID NO: 1 - DNA, 12 bp, Artificial Sequence
  Synthetic Oligonucleotide
aaaacaacccct

SEQ ID NO: 2 - DNA, 12 bp, Artificial Sequence
  Synthetic Oligonucleotide
aaaacaacgctg

SEQ ID NO: 3 - DNA, 12 bp, Artificial Sequence
  Synthetic Oligonucleotide
ttttgttctgcc

SEQ ID NO: 4 - DNA, 12 bp, Artificial Sequence
  Synthetic Oligonucleotide
ttttgttggcta

SEQ ID NO: 5 - DNA, 11 bp, Artificial Sequence
  Synthetic Oligonucleotide
tcatgtttaac

SEQ ID NO: 6 - DNA, 11 bp, Artificial Sequence
  Synthetic Oligonucleotide
atacgcccacg

SEQ ID NO: 7 - DNA, 11 bp, Artificial Sequence
  Synthetic Oligonucleotide
atacggccacg

SEQ ID NO: 8 - DNA, 11 bp, Artificial Sequence
  Synthetic Oligonucleotide
gcttagcacac

SEQ ID NO: 9 - DNA, 11 bp, Artificial Sequence
  Synthetic Oligonucleotide
gcttatcacac

SEQ ID NO: 10 - DNA, 11 bp, Artificial Sequence
  Synthetic Oligonucleotide
tcgatcggtgc

SEQ ID NO: 11 - DNA, 11 bp, Artificial Sequence
  Synthetic Oligonucleotide
tcgattggtgc

SEQ ID NO: 12 - DNA, 11 bp, Artificial Sequence
  Synthetic Oligonucleotide
attacgaacgt

SEQ ID NO: 13 - DNA, 11 bp, Artificial Sequence
  Synthetic Oligonucleotide
attacaaacgt

SEQ ID NO: 14 - DNA, 12 bp, Artificial Sequence
  Synthetic Oligonucleotide
ccagtacgaatg

SEQ ID NO: 15 - DNA, 12 bp, Artificial Sequence
  Synthetic Oligonucleotide
gacaagtacact

SEQ ID NO: 16 - DNA, 12 bp, Artificial Sequence
  Synthetic Oligonucleotide
  misc_feature (1)..(12): N = a or g or c or t/u, unknown, or other
nnnnnnnnnnnn

SEQ ID NO: 17 - DNA, 20 bp, Artificial Sequence
  Synthetic Oligonucleotide
gacgtgtgcttacccaaagg

SEQ ID NO: 18 - DNA, 25 bp, Artificial Sequence
  Synthetic Oligonucleotide
gactagccttattttaacttgctat

SEQ ID NO: 19 - DNA, 83 bp, Artificial Sequence
  Synthetic Oligonucleotide
actggctattcattcgcctcctgttcgacagtcagccgcatctgcgtctatttagtggagcccttgtgttcaatcaacattcc

SEQ ID NO: 20 - DNA, 84 bp, Artificial Sequence
  Synthetic Oligonucleotide
gttttagagctagaaatagcctcctgttcgacagtcagccgcatctgcgtctatttagtggagcccttgaaggacgaaacaccg

SEQ ID NO: 21 - DNA, 60 bp, Artificial Sequence
  Synthetic Oligonucleotide
ttcgacagtcagccgcatctgcgtctatttagtggagcccttgtgttcaatcaacattcc

SEQ ID NO: 22 - DNA, 50 bp, Artificial Sequence
  Synthetic Oligonucleotide
tcagccgcatctgcgtctatttagtggagcccttgaaggacgaaacaccg